(19) United States
(12) Patent Application Publication
(10) Pub. No.: US 2005/0238627 A1
(43) Pub. Date: Oct. 27, 2005

(54) Methods and compositions for hybrid cell vaccines for the treatment and prevention of cancer

(76) Inventors: Tsuneya Ohno, Boston, MA (US); Donald W. Kufe, Boston, MA (US)

Correspondence Address: JONES DAY, 222 EAST 41ST ST, NEW YORK, NY, 10017, US

(21) Appl. No.: 11/071,862

(22) Filed: Mar. 2, 2005

Related U.S. Application Data
(60) Provisional application No. 60/549,888, filed on Mar. 2, 2004.

(51) Int. Cl. A61K 48/00

(52) U.S. Cl. 424/93.21; 514/44

(57) ABSTRACT

The present invention relates to methods for treating and preventing cancer and for treating precancerous lesions by administering a therapeutically effective dose of a vaccine comprising fusion cells formed by fusion of antigen presenting cells and non-dendritic cells that contain genomic DNA or cDNA derived from a tumor cell or a pre-cancerous cell to a cancer patient or patient with a precancerous lesion. In certain embodiments, such vaccines are administered in combination with a cytokine or other molecule that stimulates a cytotoxic T cell (CTL) response and/or a humoral immune response. The present invention also relates to methods for treating and preventing an infectious disease by administering a therapeutically effective dose of a vaccine comprising fusion cells formed by fusion of antigen presenting cells and non-dendritic cells that contain genomic DNA or cDNA derived from the infectious agent that causes the infectious disease to be treated or prevented to a subject. The present invention also related to methods for producing the fusion cells to be used with the methods of the invention. The present invention also provides compositions comprising the fusion cells to be used with the methods of the invention.

### RELATED APPLICATIONS

This application claims benefit of priority of U.S. provisional application No. 60/549,888 filed on Mar. 2, 2004, which is incorporated herein by reference in its entirety.

### 1. INTRODUCTION

The present invention relates to methods for treating and preventing cancer and for treating precancerous lesions by administering a therapeutically effective dose of a vaccine comprising fusion cells formed by fusion of antigen presenting cells and non-dendritic cells that contain genomic DNA or cDNA derived from a tumor cell or a pre-cancerous cell to a cancer patient or patient with a precancerous lesion. In certain embodiments, such vaccines are administered in combination with a cytokine or other molecule that stimulates a cytotoxic T cell (CTL) response and/or a humoral immune response. The present invention also relates to methods for treating and preventing an infectious disease by administering a therapeutically effective dose of a vaccine comprising fusion cells formed by fusion of antigen presenting cells and non-dendritic cells that contain genomic DNA or cDNA derived from the infectious agent that causes the infectious disease to be treated or prevented to a subject. The present invention also related to methods for producing the fusion cells to be used with the methods of the invention. The present invention also provides compositions comprising the fusion cells to be used with the methods of the invention. The invention also provide universal antigen presenting cells and universal antigen presenting cells containing genomic DNA, cDNA, or mRNA derived from a tumor cell, cell of a precancerous lesion, or infectious agent. The invention further provides methods for administering such universal antigen presenting cells to a subject.

### 2. BACKGROUND OF THE INVENTION

There is great interest in the development of an effective immunotherapeutic composition for preventing cancer. Success at such an immunotherapeutic approach will require the development of a composition that is both capable of eliciting a very strong immune response, that is extremely specific for the target tumor or infected cell.

### 2.1 THE IMMUNE RESPONSE

Cells of the immune system arise from pluripotent stem cells through two main lines of differentiation, the lymphoid lineage and the myeloid lineage. The lymphoid lineage produces lymphocytes, such as T cells, B cells, and natural killer cells, while the myeloid lineage produces monocytes, macrophages, and neutrophils and other accessory cells, such as dendritic cells, platelets, and mast cells. There are two main types of T cells of the lymphoid lineage, cytotoxic T lymphocytes (CTLs) and helper T cells which mature and undergo selection in the thymus, that are distinguished by the presence of one of two surface markers, CD8 (CTLs) or CD4 (helper T cells).

Lymphocytes circulate and search for invading foreign pathogens and antigens that tend to become trapped in secondary lymphoid organs, such as the spleen and the lymph nodes. Antigens are taken up in the periphery by the antigen-presenting cells (APCs) that migrate to secondary lymphoid organs. Interaction between T cells and APCs triggers several effector pathways, including activation of B cells and antibody production, activation of CD8⁺ cytotoxic T lymphocytes (CD8⁺ CTLs), and stimulation of cytokine production by T cells.

Activation of naive B cells, to produce antibodies, requires two signals:

(1) recognition and binding of specific antigens by surface-bound receptors (B cell receptors, or BCR), which then cluster together along with BCR-associated signaling molecules, and

(2) a co-stimulatory signal provided by binding of the CD40 receptor on the B cell surface by the CD40L ligand carried on the surface of activated T-helper cells (Th). Activated B cells undergo clonal expansion, somatic hypermutation, affinity maturation, and isotype switching, in which the heavy chain class of the secreted antibody is established. Selection of the antibody heavy-chain class, in turn, is determined by the collection of cytokines contacting the B cell at the time isotype switching is carried out.

The heavy-chain constant region (Fc) of an antibody influences the function of that antibody in vivo. For example, the Fc portion of the IgG class of antibodies is recognized and bound by cell-surface receptors of professional phagocytic cells such as macrophage and neutrophils, thereby facilitating ingestion and destruction of IgG-bound antigens and/or cells opsonized in this manner. In addition, clusters of IgG antibodies bound, e.g., to multiple copies of a cell-surface antigen will fix and activate the complement system, leading to the destruction of that cell.

In contrast to antigen recognition and binding by BCR and antibodies, T cells require that antigenic proteins be processed by one of two distinct routes, depending upon whether the origin of the antigen is intracellular or extracellular, and presented as part of a cell-surface-bound complex. Intracellular or endogenous protein antigens are presented to CD8⁺ CTLs by class I major histocompatibility complex (MHC) molecules that are expressed in most cell types, including tumor cells. Extracellular antigenic determinants are presented on the cell surface of “specialized” or “professional” APCs, such as dendritic cells and macrophages, as class II MHC molecules-antigen complexes that are recognized by CD4⁺ “helper” T cells (see generally, W. E. Paul, ed., Fundamental Immunology. New York: Raven Press, 1984).

Class I and class II MHC molecules are the most polymorphic proteins known. A further degree of heterogeneity of MHC molecules is generated by the combination of class I and class II MHC molecules, known as the MHC haplotype. In humans, HLA-A, HLA-B and HLA-C, three distinct genetic loci located on a single chromosome, encode class I molecules. Because T cell receptors specifically bind complexes comprising an antigenic peptide and the polymorphic portion of an MHC molecule, T cells respond poorly when an MHC molecule of a different genetic type is encountered. This specificity results in the phenomenon of MHC-restricted T cell recognition and T cell cytotoxicity.

Lymphocytes circulate in the periphery and become “primed” in the lymphoid organs on encountering the appropriate signals (Bretscher and Cohn, 1970, Science 169:1042-1049). The first signal is received through the T cell receptor after it engages antigenic peptides displayed by class I MHC molecules on the surface of APCs. The second signal is provided either by a secreted chemical signal or cytokine, such as interleukin-1 (IL-1), interferon-γ, interleukin-2 (IL-2), interleukin-4 (IL-4), interleukin-7 (IL-7), and interleukin-12 (IL-12), produced by CD4⁺ helper T cells or dendritic cells, or by a plasma-membrane-bound co-stimulatory molecule, such as B7 (a term which includes B7.1 and B7.2 molecules), which is present on the antigen-presenting-cell membrane and is recognized by a co-receptor on the cell surface of helper T cells, called CD28, a member of the Ig superfamily. Interferon-γ and IL-12 production are associated with the helper T cell subtype known as TH₁ that promote development of CD8⁺ T cells, and IL-4 production, which is associated with the T helper cell subtype known as TH₂ that promotes development and activation of antibody-producing B cells.

In addition to antigen-specific interactions during antigen presentation, antigen non-specific adhesive that stabilize binding of T lymphocytes to APC are also involved in T cell stimulation. More specifically, receptor molecules on APC, such as ICAM-1/CD54, LFA-3/CD58, and B7, bind corresponding co-receptors on T cells. Helper T cells receiving both signals are activated to proliferate and to secrete a variety of interleukins. CTLs receiving both signals are activated to kill target cells that carry the same class I MHC molecule and the same antigen that originally induced CTL activation. Accordingly, CD8⁺ CTLs are important in resisting cancer and pathogens, as well as rejecting allografts (Terstappen et al., 1992, Blood 79:666-677). However, T cells receiving the first signal in the absence of co-stimulation become anergized, leading to tolerance (Lamb et al., 1983, J. Exp. Med. 157:1434-1447; Mueller et al., 1989, Annu. Rev. Immunol. 7:445-480; Schwartz, 1992, Cell 71:1065-1068; Mueller and Jenkins, 1995, Curr. Opin. Immunol. 7:375-381).

### 2.2 PATHOBIOLOGY OF CANCER

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor pre-neoplastic changes, which may under certain conditions progress to neoplasia. Therefore, during the progression of this multistep process, pre-cancerous cells accumulate that comprise at least one genetic allele that distinguishes a pre-cancerous cell from a normal cell. Such genetic differences can result in the expression of tumor-specific antigens, over-expression of normal cellular proteins, and/or altered cellular distribution of normal and/or tumor-specific antigens. In certain instances, these alterations may result in cell-surface expression of an altered cell-surface protein or of a normal protein that is generally not transported to the cell surface.

Accumulation of pre-cancerous cells is detected as pre-malignant abnormal cell growth that is exemplified by hyperplasia, metaplasia, or most particularly, dysplasia (for a review of such abnormal growth conditions, see Robbins and Angell, 1976, Basic Pathology, 2d. Ed., W. B. Saunders Co., Philadelphia, pp. 68-79). Hyperplasia is a form of controlled cell proliferation involving an increase in cell number in a tissue or organ, without significant alteration in structure or function. One example of hyperplasia is endometrial hyperplasia, which often precedes endometrial cancer. Metaplasia is a form of controlled cell growth in which one type of adult cell or fully-differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic growth involving a loss individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The neoplastic lesion, which comprises the pre-cancerous and cancerous cells described above, may evolve clonally as pre-cancerous cells accumulation a plurality of genetic alterations that provide an increasing capacity for invasion, growth, metastasis, and heterogeneity, especially under conditions in which the neoplastic cell escapes the host is immune surveillance (Roitt, I., Brostoff, J., and Kale, D., 1993, Immunology, 3^{rd} Ed., Mosby, St. Louis, pps. 17.1-17.12).

### 2.3 IMMUNOTHERAPY AGAINST CANCER

The cytotoxic T cell response is a very important host response for the control of growth of antigenic tumor cells (Anichimi et al., 1987, Immunol. Today 8:385-389). Studies with experimental animal tumors as well as spontaneous human tumors have demonstrated that many tumors express antigens that can induce an immune response. Some antigens are unique to the tumor, and some are found on both tumor and normal cells. Several factors influence the immunogenicity of the tumor, including, for example, the specific type of carcinogen involved, and immunocompetence of the host and the latency period (Old et al., 1962, Ann. N. Y. Acad. Sci. 101:80-106; Bartlett, 1972, J. Natl. Cancer. Inst. 49:493-504). It has been demonstrated that T cell-mediated immunity is of critical importance for rejection of virally and chemically induced tumors (Klein et al., 1960, Cancer Res. 20:1561-1572; Tevethia et al., 1974, J. Immunol. 13:1417-1423).

Adoptive immunotherapy for tumors refers to the therapeutic approach wherein immune cells with antitumor activity are administered to a tumor-bearing host, with the objective that the cells cause regression of an established tumor, either directly or indirectly. Immunization of hosts bearing established tumors with tumor cells or tumor antigens, as well a spontaneous tumors, has often been ineffective since the tumor may have already elicited an immunosuppressive response (Greenberg, 1987, Chapter 14, in Basic and Clinical Immunology, 6th ed., ed. by Stites, Stobo and Wells, Appleton and Lange, pp. 186-196; Bruggen, 1993). Thus, prior to immunotherapy, it had been necessary to reduce the tumor mass and deplete all the T cells in the tumor-bearing host (Greenberg et al., 1983, page 301-335, in “Basic and Clinical Tumor Immunology”, ed. Herbermann R R, Martinus Nijhoff).

Animal models have been developed in which hosts bearing advanced tumors can be treated by the transfer of tumor-specific syngeneic T cells (Mulé et al., 1984, Science 225:1487-1489). Investigators at the National Cancer Institute (NCI) have used autologous reinfusion of peripheral blood lymphocytes or tumor-infiltrating lymphocytes (TIL), T cell cultures from biopsies of subcutaneous lymph nodules, to treat several human cancers (Rosenberg, S. A., U.S. Pat. No. 4,690,914, issued Sep. 1, 1987; Rosenberg et al., 1988, N. Engl. J. Med., 319:1676-1680). For example, TIL expanded in vitro in the presence of IL-2 have been adoptively transferred to cancer patients, resulting in tumor regression in select patients with metastatic melanoma. Melanoma TIL grown in IL-2 have been identified as CD3⁺-activated T lymphocytes, which are predominantly CD8⁺ cells with unique in vitro anti-tumor properties. Many long-term melanoma TIL cultures lyse autologous tumors in a specific class I MHC-antigen complex and T cell receptor-dependent manner (Topalian et al., 1989, J. Immunol. 142:3714).

Application of these methods for treatment of human cancers would entail isolating a specific set of tumor-reactive lymphocytes present in a patient, expanding these cells to large numbers in vitro, and then putting these cells back into the host by multiple infusions. Since T cells expanded in the presence of IL-2 are dependent upon IL-2 for survival, infusion of IL-2 after cell transfer prolongs the survival and augments the therapeutic efficacy of cultured T cells (Rosenberg et al., 1987, N. Engl. J. Med. 316:889-897). However, the toxicity of the high-dose IL-2 and activated lymphocyte treatment has been considerable, including high fevers, hypotension, damage to the endothelial wall due to capillary leak syndrome, and various adverse cardiac events such as arrhythmia and myocardial infarction (Rosenberg et al., 1988, N. Engl. J. Med. 319:1676-1680). Furthermore, the demanding technical expertise required to generate TILs, the quantity of material needed, and the severe adverse side effects limit the use of these techniques to specialized treatment centers.

CTLs specific for class I MHC-peptide complexes could be used in treatment or prevention of cancer, and ways have been sought to generate such CTLs in vitro without the requirement for priming in vivo. These include the use of dendritic cells pulsed with appropriate antigens (Inaba et al., 1987, J. Exp. Med. 166:182-194; Macatonia et al., 1989, J. Exp. Med. 169:1255-1264; De Bruijn et al., 1992, Eur. J. Immunol. 22:3013-3020). RMA-S cells (mutant cells expressing high numbers of “empty” cell surface class I MHC molecules) loaded with peptide (De Bruijn et al., 1991, Eur. J. Immunol. 21:2963-2970; De Bruijn et al., 1992, supra; Houbiers et al., 1993, Eur. J. Immunol. 26:2072-2077) and macrophage phagocytosed-peptide loaded beads (De Bruijn et al., 1995, Eur. J. Immunol. 25, 1274-1285).

Fusion of B cells or dendritic cells with tumor cells has been previously demonstrated to elicit anti-tumor immune responses in animal models (Guo et al., 1994, Science, 263:518-520; Stuhler and Walden, 1994, Cancer Immunol. Immuntother. 1994, 39:342-345; Gong et al., 1997, Nat. Med. 3:558-561; Celluzzi, 1998, J. Immunol. 160:3081-3085; Gong, PCT publication WO 98/46785, dated Oct. 23, 1998). In particular, immunization with hybrids of tumor cells and antigen presenting cells has been shown to result in protective immunity in various rodent models.

However, the current treatments, while stimulating protective immunity, do not always effectively treat a patient who already has an established disease, namely, the administration of fusion cells to a subject with a disease, does not always stimulate an immune response sufficient to eliminate the disease. Such treatments are generally not effective for prevention of cancer in those patients who, although they may be tumor-free, nevertheless carry pre-cancerous lesions. Thus, a need exists for a therapeutic composition which can be used for prevention of neoplastic disease, prevent recurrence of neoplastic disease, and cause the regression of an existing tumor in a patient. Moreover, there is an especially acute need for such compositions for the prophylactic treatment of those patients known to carry one or more genetic markers or alleles that are strongly predictive of an eventual development of neoplastic disease.

Further, current treatments are limited by the availability of tumor cells for the generation of the fusion cells. The availability of tumor cells may be particularly problematic if autologous tumor cells from the subject to be treated are to be used for the generation of the fusion cells and if the surgical removal of such tumor cells in contraindicated. Sufficient amounts of tumor cells may in some instances only be available if the patient's tumor cells are expanded in culture, which may be too time consuming to provide the patient with the full benefit of the treatment. Thus, a need exists for methods of generating fusion cells for adoptive immunotherapy from smaller numbers of tumor cells.

Citation or discussion of a reference herein shall not be construed as an admission that such is prior art to the present invention.

### 3. SUMMARY OF THE INVENTION

The present invention relates to methods for preventing cancer by administration of fusion cells formed by fusion of antigen presenting cells, such as dendritic cells, and non-dendritic cells that contain genomic DNA extracted from a tumor cell or a pre-cancerous cell, which fusion cells may also be administered in combination with a molecule which stimulates a CTL and/or humoral immune response. The invention is based, in part, on the discovery and demonstration that administration of fusion cells formed by fusion of antigen presenting cells, such as dendritic cells, and non-dendritic cells that contain genomic DNA extracted from a tumor cell results in a potentiated immune response against development of that cancer, as well as in treatment and prevention of that cancer. Such fusion cells combine the vigorous immunostimulatory effect of dendritic cells with the specific antigenicity of the tumor cells from which the genomic DNA was extracted, thereby eliciting a strong, specific immune response, which can further be enhanced by the co-administration of an immune activator.

The instant invention provides for administration of fusion cells formed by fusion of antigen presenting cells and non-dendritic cells that contain genomic DNA extracted from a tumor cell or a precancerous cell, as well as the co-administration of fusion cells formed by fusion of antigen presenting cells and non-dendritic cells that contain genomic DNA extracted from a tumor cell or a precancerous cell, with a cytokine or other molecule which stimulates a CTL and/or humoral immune response, thereby significantly enhancing the effectiveness of the therapeutic treatment.

In one embodiment, the invention provides a method of preventing cancer in a mammal, which comprises administering to a mammal in need of such prevention a therapeutically effective amount of fusion cells formed by fusion of antigen presenting cells and non-dendritic cells that contain genomic DNA extracted from a tumor cell or a precancerous cell. In a preferred embodiment, the fusion cells are administered in combination with a molecule which stimulates a CTL and/or humoral immune response. In another aspect of this embodiment, the co-stimulator of a CTL and/or humoral immune response is also provided by transforming or transfecting the fusion cells with genetic material that encodes the co-stimulator.

In another embodiment, the invention provides a method of preventing cancer in a mammal, said method comprising administering to a mammal in need of said prevention an effective amount of fusion cells, wherein a fusion cell (i) is formed by fusion of antigen presenting cells and non-dendritic cells that contain genomic DNA extracted from a tumor cell and (ii) shares at least one MHC class I allele with said mammal, and wherein said non-dendritic cell that comprises genomic DNA extracted from a tumor cell displays at least one antigen having the antigenicity of an antigen associated with said cancer. In a more specific embodiment, the antigen is specific to said cancer. In a specific embodiment of this method, the non-dendritic cell comprises genomic DNA from a tumor cell that is of the same cell type as the cell type that constitutes the cancer that is to be prevented or treated. In another specific embodiment, the method further comprises administration of a molecule that stimulates a humoral immune response or a cytotoxic T cell immune response. In one embodiment, said molecule is a cytokine. In one embodiment, the cytokine is interleukin-12. In another embodiment, the dendritic cell is obtained from human blood monocytes. In another embodiment, said non-dendritic cell is obtained from a primary culture of non-dendritic cells derived from said mammal. In another embodiment, the tumor cell is obtained from a primary culture of tumor cells derived from said mammal. In another embodiment, said antigen presenting cells are autologous to said mammal. In another embodiment, said antigen presenting cells are allogeneic to the mammal. In another embodiment, said antigen presenting cells are allogeneic to the mammal and wherein said non-dendritic cells have the same class I MHC haplotype as the mammal. In certain embodiments, the antigen presenting cell is a universal antigen presenting cell (see section 4.7). In another embodiment, the mammal is a human. In another embodiment, the mammal is selected from the group consisting of a cow, a horse, a sheep, a pig, a fowl, a goat, a cat, a dog, a hamster, a mouse and a rat.

In another embodiment of this method, the cancer to be treated or prevented is selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenstrom's macroglobulinemia, and heavy chain disease.

In another embodiment, the invention provides a method of treating a pre-cancerous lesion in a mammal, said method comprising administering to a mammal in need of said treatment a therapeutically effective amount of fusion cells, wherein the fusion cells (i) are formed by fusion of antigen presenting cells and non-dendritic cells that contain genomic DNA extracted from a pre-cancerous cell and (ii) share at least one MHC class I allele with said mammal. In certain embodiments, said non-dendritic cell that contains genomic DNA of a precancerous cell displays at least one antigen having the antigenicity of an antigen associated with said pre-cancerous lesion. In a more specific embodiment, the antigen is specific to said pre-cancerous lesion. In a specific embodiment, the pre-cancerous cell from which the genomic DNA is extracted is of the same cell type as the cell type that constitutes the pre-cancerous lesion. In another specific embodiment, said precancerous cell is isolated from said pre-cancerous lesion. In another specific embodiment, the method further comprises administration of a molecule that stimulates a humoral immune response or a cytotoxic T cell immune response. In one embodiment, said molecule is a cytokine. In one embodiment, the cytokine is interleukin-12. In another embodiment, the dendritic cell is obtained from human blood monocytes. In another embodiment, said non-dendritic cell is obtained from a primary culture of non-dendritic cells derived from said mammal. In another embodiment, said pre-cancerous cell from which the genomic DNA is extracted is obtained from a primary culture of pre-cancerous cells derived from said mammal. In another embodiment, said antigen presenting cells are autologous to said mammal. In another embodiment, said antigen presenting cells are allogeneic to the mammal. In another embodiment, said antigen presenting cells are allogeneic to the mammal and wherein said non-dendritic cells have the same class I MHC haplotype as the mammal. In certain embodiments, the antigen presenting cell is a universal antigen presenting cell (see section 4.7). In another embodiment, mammal is a human. In another embodiment, the mammal is selected from the group consisting of a cow, a horse, a sheep, a pig, a fowl, a goat, a cat, a dog, a hamster, a mouse and a rat.

In another embodiment, said pre-cancerous lesion is a precursor of a cancer selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenström's macroglobulinemia, and heavy chain disease.

The invention further encompasses a method for fusing human antigen presenting cells and non-dendritic human cells that comprise genomic DNA extracted from a tumor cell or a pre-cancerous cell comprising subjecting a population of antigen presenting cells and a population of non-dendritic cells to conditions that promote cell fusion. In one embodiment, said non-dendritic cells are autologous to said antigen presenting cells. In another embodiment, the cell fusion is accomplished by electrofusion. In another embodiment, the method further comprising the step of inactivating the population of fusion cells. In another embodiment, the inactivating the population of fusion cells is accomplished by y irradiating the cells. In certain embodiments, the tumor cells or cells of a pre-cancerous lesion are inactivated by γ irradiation before extraction of genomic DNA or mRNA to avoid any contamination with active tumor cells or cells of a pre-cancerous lesion.

The invention further provides a kit comprising, in one or more containers, a population of antigen presenting cells, a population of non-dendritic cells and instructions for transfecting genomic DNA of a tumor cell or a pre-cancerous cell into the non-dendritic cell and for fusing said antigen presenting cells with non-dendritic cells for administration to a mammal in need thereof. In one embodiment, the kit further comprises a molecule that stimulates an immune response selected from the group consisting of humor immune responses, cytotoxic T cell responses, and combinations thereof, and instructions for use of the kit for preventing or treating cancer. In one embodiment, the molecule is a cytokine. In another embodiment, the cytokine is IL-12. In another embodiment, the kit further comprises a cuvette suitable for electrofusion. In another embodiment, the antigen presenting cells are cryopreserved.

In another embodiment, the invention provides a pharmaceutical composition comprising a fusion cell comprising a dendritic cell fused to a non-dendritic cell that comprises genomic DNA extracted from a tumor cell or a pre-cancerous cell. In one embodiment, the non-dendritic cell is freshly isolated or obtained from a primary cell culture. In certain embodiments, the tumor cell or the pre-cancerous cell is obtained from a primary cell culture. In another embodiment, the pharmaceutical composition further comprises a molecule that stimulates an immune response selected from the group consisting of humor immune responses, cytotoxic T cell responses, and combinations thereof. In another embodiment, the molecule is a cytokine. In another embodiment, the molecule is IL-12. In another embodiment, the dendritic cell is autologous to the mammal. In another embodiment, the non-dendritic cell is autologous to the to the mammal. In another embodiment, the tumor cell or the pre-cancerous cell is obtained from the subject that is to be treated. In another embodiment, the dendritic cell is a human cell. In another embodiment, the non-dendritic cell is a human cell. In another embodiment, the tumor cell or the pre-cancerous cell or the tumor cell is of the same cell type as the cell type that constitutes the cancer or the pre-cancerous lesion to be prevented. In another embodiment, the pre-cancerous cell or the tumor cell is the same cell type as the pre-cancerous lesion or the cancer to be treated. In another embodiment, the pre-cancerous cell is isolated from a pre-cancerous lesion autologous to the mammal, and wherein the pre-cancerous lesion is a precursor of a cancer to be prevented. In another embodiment, the pre-cancerous cell is isolated from a pre-cancerous lesion of the mammal that is to be treated with said composition.

In another embodiment, the invention provides for fusion cells comprising a dendritic cell that is fused to a non-dendritic cell that comprises genomic DNA extracted from a tumor cell or a pre-cancerous cell. In a preferred embodiment, the dendritic, the non-dendritic cell, and the tumor cell or the pre-cancerous cell are human. The present invention also encompasses a population of such fusion cells, wherein at least 10%-15% of the cells are fused, and preferably 20%-30% of the cells are fused. In certain embodiments, at least 10%, at least 20%, at least 30%, at least 40%, at least 50%, at least 60%, at least 70%, at least 80%, at least 90%, or at least 95% of the cells are fused. In certain embodiments, at most 10%, at most 20%, at most 30%, at most 40%, at most 50%, at most 60%, at most 70%, at most 80%, at most 90%, or at most 95% of the cells are fused.

As used herein, a compound, such as a cytokine, is said to be “co-administered” or administered in “combination” with another compound, such as a fusion cell, when either the physiological effects of both compounds, or the elevated serum concentration of both compounds can be measured simultaneously. With compounds that increase the level of endogenous cytokine production, the serum concentration of the endogenously produced cytokine and the other administered agent (ie., fusion cell), can also be measured simultaneously when “co-administered” or in “combination”. Thus, compounds may be administered either simultaneously, as separate or mixed compositions, or they may be administered sequentially provided that an elevation of their levels in serum can be measured simultaneously at some point during administration.

Unless otherwise stated the terms “combination therapy” and “combination treatments” are used herein to describe a therapeutic regimen involving co-administration of the subject fusion cells and a molecule which stimulates a CTL response and/or humoral immune response, which results in preventing cancer, which can be measured, for example, by demonstration of a reduction in the number of tumor cells that form, or by the failure to develop pre-cancerous lesions or tumors in a patient genetically predisposed to do so, and by the failure, or reduced rate of progression, of one or more pre-cancerous lesions to develop into tumors.

In another embodiment, the invention provides a kit comprising, in one or more containers, a sample containing a population of antigen presenting cells and instructions for its use in preventing cancer. In another embodiment, the kit further comprising a cuvette suitable for electrofusion. In another embodiment, the antigen presenting cells are cryopreserved. In a further embodiment, the kit comprises a molecule that stimulates a humoral immune response and/or a cytotoxic T cell response. In a more preferred embodiment the stimulatory molecule is a cytokine such as, but not limited to interleukin-12.

The methods of the invention can be used to treat and/or prevent a tumor, cancer, neoplastic disease, and/or precancerous lesion. In certain embodiments, the methods of the invention are used to inhibit or reduce the growth of a cancer cell, a neoplastic cell, or a cell of a precancerous lesion in a patient. In certain embodiments, the methods of the invention are used to stimulate or to augment the immune response in a patient against the cancer or the neoplastic disease that is to be treated in the patient.

In other embodiments, the methods of the invention relate to the treatment and prevention of an infectious disease. The methods of the invention for treating or preventing an infectious disease comprise administering fusion cells to the subject in which the infectious disease is to be treated or prevented, wherein the fusion cells are generated by fusing antigen presenting cells with non-dendritic cells that comprise genomic DNA extracted from an infectious agent or from a cell infected with an infectious agent. Different non-dendritic cells may be used with the methods of the invention. In a preferred embodiment, the non-dendritic cells are derived from the subject that is to be treated. In certain embodiments, the fusion cells comprise at least one MHC class I allele that is identical to an MHC class I allele of the subject that is to be treated. In a preferred embodiment, the genomic DNA contains genomic DNA from the same species of infectious agent with which the subject that is to be treated is infected or is at risk of being infected with. In a more specific embodiment, the infectious agent is obtained from the subject to be treated.

The present invention further provides methods for preventing cancer by administration of fusion cells formed by fusion of antigen presenting cells, such as dendritic cells, and non-dendritic cells that contain cDNA derived from a tumor cell or a pre-cancerous cell, which fusion cells may also be administered in combination with a molecule which stimulates a CTL and/or humoral immune response. The present invention also provides methods for treating or preventing an infectious disease by administration of fusion cells formed by fusion of antigen presenting cells, such as dendritic cells, and non-dendritic cells that contain cDNA derived from an infectious agent that causes the infectious disease or a cell that is infected with the infectious agent, which fusion cells may also be administered in combination with a molecule which stimulates a CTL and/or humoral immune response.

In certain embodiments, the invention provides a method of treating or preventing cancer in a mammal, said method comprising administering to a mammal in need of said treatment or prevention an effective amount of universal antigen presenting cells, wherein a universal antigen presenting cell (i) has been engineered to recombinantly express one or more costimulatory molecules selected from the group consisting of: ICAM-I, ICAM-II, B7, and LFA-3; (ii) comprises genomic DNA of a cancer cell and wherein said genomic DNA encodes at least one antigen having the antigenicity of an antigen associated with said cancer; and (iii) shares at least one MHC class I allele with said mammal.

In certain embodiments, the invention provides a method of treating or preventing cancer in a mammal, said method comprising administering to a mammal in need of said treatment or prevention an effective amount of fusion cells, wherein a fusion cell (i) is formed by the fusion of an antigen presenting cell and a non-dendritic cell, wherein the non-dendritic cell comprises one or more cDNAs wherein at least one cDNA encodes an antigen having the antigenicity of an antigen associated with said cancer, and (ii) shares at least one MHC class I allele with said mammal.

In certain embodiments, the invention provides a method of treating or preventing cancer in a mammal, said method comprising administering to a mammal in need of said treatment or prevention an effective amount of universal antigen presenting cells, wherein a universal antigen presenting cell (i) has been engineered to recombinantly express one or more costimulatory molecules selected from the group consisting of: ICAM-I, ICAM-II, B7, and LFA-3; (ii) comprises one or more cDNAs wherein at least one cDNA encodes an antigen having the antigenicity of an antigen associated with said cancer, and (iii) shares at least one MHC class I allele with said mammal.

In certain embodiments, the invention provides a method for fusing human antigen presenting cells and non-dendritic human cells comprising subjecting a population of antigen presenting cells and a population of non-dendritic cells to conditions that promote cell fusion, wherein the non-dendritic cells comprise one or more cDNAs wherein at least one cDNA encodes an antigen associated with a cancer.

In certain embodiments, the invention provides a fusion cell of an antigen presenting cell and a non-dendritic cell, wherein the fusion cell comprises a cDNA encoding an antigen associated with a tumor cell.

In certain embodiments, the invention provides a kit comprising, in one or more containers, (i) a population of antigen presenting cells; (ii) a population of non-dendritic cells; and (iii) instructions for fusing said antigen presenting cells with the non-dendritic cells for administration to a mammal in need thereof.

In certain embodiments, the invention provides a pharmaceutical composition comprising a fusion cell comprising an antigen presenting cell and a non-dendritic cell, wherein the non-dendritic cell comprises at least one cDNA encoding an antigen associated with a tumor cell.

### 3.1 BRIEF DESCRIPTION OF THE FIGURES

FIG. 1 shows the tumor volume of B16 bearing mice and MC38 bearing mice, respectively, after vaccination with fusion cells. NIH/B16 designates fusion cells of antigen presenting cells and NIH3T3 fibroblasts transfected with genomic DNA extracted from B16 tumor cells. NIH3T3 designates fusion cells of non-transfected NIH3T3 fibroblasts and dendritic cells. Day 0 is the day of challenge with the tumor.

FIG. 2 shows the tumor volume of B16 and MC38, respectively, bearing mice after vaccination with fusion cells. NIH/B16 designates fusion cells of dendritic cells and NIH3T3 fibroblasts transfected with genomic DNA extracted from B16 tumor cells. NIH3T3 designates fusion cells of non-transfected NIH3T3 fibroblasts with dendritic cells. NIH/CT2A designates fusion cells of dendritic cells and NIH3T3 fibroblasts transfected with genomic DNA extracted from CT2A tumor cells. NIH/B16DNase designates fusion cells of dendritic cells and NIH3T3 fibroblasts transfected with heat-treated genomic DNA extracted from B16 tumor cells. Day 0 is the day of challenge with the tumor.

FIG. 3 shows the tumor volume of B16 tumors after vaccination with fusion cells. NIH/B16*1 designates fusion cells of dendritic cells and NIH3T3 fibroblasts transfected with 1× of genomic DNA extracted from B16 tumor cells. NIH/B16*1/10 designates fusion cells of dendritic cells and NIH3T3 fibroblasts transfected with 0.1× of genomic DNA extracted from B16 tumor cells. NIH/B16*1/100 designates fusion cells of dendritic cells and NIH3T3 fibroblasts transfected with 0.01× of genomic DNA extracted from B16 tumor cells. NIH3T3 designates fusion cells of non-transfected NIH3T3 and dendritic cells. Day 0 is the day of challenge with the tumor.

FIG. 4 shows the tumor volume of B16 tumors after treatment with fusion cells. NIH/B16 designates fusion cells of dendritic cells and NIH3T3 fibroblasts transfected with genomic DNA extracted from B16 tumor cells. NIH3T3 designates fusion cells of NIH3T3 fibroblasts that were not transfected with dendritic cells and dendritic cells. Day 0 is the day of challenge with the tumor.

FIG. 5 shows tumor volume of B16 tumors after treatment with fusion cells or NIH3T3 cells transfected with genomic DNA extracted from B16 cells. N/B designates NIH3T3 cells transfected with genomic DNA extracted from B16 cells. N/B16+CD designates fusion cells of dendritic cells and NIH3T3 fibroblasts transfected with genomic DNA extracted from B16 tumor cells. Day 0 is the day of challenge with the tumor.

FIG. 6 shows tumor volume of B16 tumors after treatment with fusion. N/B16 designates fusion cells of dendritic cells and NIH3T3 fibroblasts transfected with genomic DNA extracted from B16 tumor cells. N/N designates fusion cells of dendritic cells and NIH3T3 cells transfected with DNA from NIH3T3 cells. N/denatN designates fusion cells of dendritic cells and NIH3T3 fibroblasts transfected with denatured genomic DNA extracted from B16 tumor cells. Day 0 is the day of challenge with the tumor.

FIG. 7 shows the percentage of specific lysis of B16 cells by splenocytes that were isolated from mice that were treated with different fusion cells. NIH/B16-1 designates fusion cells of dendritic cells and NIH3T3 fibroblasts transfected with genomic DNA extracted from B16 tumor cells. In this assay, the splenocytes of the mice that were treated with NIH/B16 were tested for their cytotoxicity against B16 cells. NIH/B16DNase designates fusion cells of dendritic cells and NIH3T3 fibroblasts transfected with heat-treated genomic DNA extracted from B16 tumor cells. NIH/CT2A designates fusion cells of dendritic cells and NIH3T3 fibroblasts transfected with genomic DNA extracted from CT2A tumor cells. NIH3T3 designates fusion cells of NIH3T3 fibroblasts that were not transfected with dendritic cells and dendritic cells. NIH/B16-YAC1 designates fusion cells of dendritic cells and NIH3T3 fibroblasts transfected with genomic DNA extracted from B16 tumor cells. In this assay, the splenocytes of the mice that were treated with NIH/B16 were tested for their cytotoxicity against YAC1 cells.

FIG. 8 Expression of GFP. Clusters of cells expressing GFP were present among transduced cells (A). Most MC38/GFP cells were positive for GFP (B) and parental NIH3T3 cells were negative (C).

FIG. 9 Fusion efficiency. DCs and genetically-engineered fibroblasts were stained with anti-mouse CD80 monoclonal antibody and PKH-26, respectively, and fused using PEG. Double positive cells were determined to be fusion cells. A: 85% of DCs were positive for anti-CD80 monoclonal antibody. B: More than 97% of NIH/B16 cells were positive for PKH26. C: The percentage of double positive cells was 30.3%.

FIG. 10 Antitumor effects of immunization with FCs. A: Antitumor effects of prior immunization with FCs on subcutaneous tumors. FC/B16 (●), NIH/B16 cells (not fused with DCs;□), FC/CT-2A (▪), or NIH3T3 cells (∘) as a control, were injected s.c. into the flank of C57/6 mice on days 0 and 7 (n=5 in each group). On day 14, 1×10⁶ B16 cells were inoculated s.c. into the flank. The administration of FC/B16 prolonged the latency period before tumor appearance, while the administration of FC/CT-2A, NIH/B16 or NIH3T3 cells did not shorten the latency period before tumor appearance. B: We used FCs containing DCs and NIH/3T3 transfected with B16 genomic DNA digested with DNase (▴) or denatured DNA (∇) as a negative control. We also used FC/NIH (□). Immunization with these FCs did not shorten the latency period before tumor appearance. C: NIH/3T3 cells were transfected with 2 (●), 0.2 (▪), or 0.02 (∇) μg of genomic DNA from B16 cells. FCs containing DCs and each type of NIH/3T3 were identified as FC/high, FC/mid, and FC/low, respectively. No difference in antitumor effects was observed in response to immunization with FC/low or NIH3T3 (∘), whereas immunization with FC/high or FC/mid remarkably inhibited the growth of subcutaneous tumors.

FIG. 11 Cytotoxicity of spleen cells from tumor-bearing mice. SPCs were separated from mice injected with FC/B16 (●, ∘), mice injected with FCs containing DCs and NIH/3T3 transfected with B16 genomic DNA digested with DNase (▴), mice injected with FC/CT-2A (▪), or mice injected with NIH3T3 cells (∇) on days 0 and 7. SPC were separated from the mice on day 14. CTL activity on B16 cells from mice immunized with FC/B16 (●) was considerably higher than in the control and other mice, and antitumor activity on Yac-1 cells from mice immunized with FC/B16 increased (∘).

FIG. 12 NK cells are required for antitumor effects of FCs. NK cells were depleted by administering anti-asialo GM1 into mice given injections of B16 cells and FCs. On days 0 and 7, FC/B16 were subcutaneously inoculated into the flank. Subsequently, on day 14, B16 cells were inoculated into the same flank. Anti-asialo GM1 was injected i.p. on days −1, 3, 7, and 10. The antitumor effect was reduced in mice depleted of NK cells compared with the controls (n=5 in each group).

FIG. 13 shows the schedule for administration of tumor cells and fusion cells for the treatment in animal studies. Fusion cells are fusion cells between dendritic cells and NIH3 cells transfected with genomic DNA from B16 tumor cells. The tumor cells are B16 cells.

FIG. 14 shows the schedule for administration of tumor cells and fusion cells for the prevention in animal studies. Fusion cells are fusion cells between dendritic cells and NIH3 cells transfected with genomic DNA from B16 tumor cells. The tumor cells are B16 cells.

FIG. 15 shows the protocol for transfecting NIH3T3 cells with genomic DNA from B16 melanoma cells.

### 3.2 ABBREVIATIONS AND CONVENTIONS

### Abbreviation

MC38 murine colon adenocarcinoma cell line
CTL Cytotoxic T lymphocytes
DC Dendritic Cells
FC Fusion Cells

As used herein, the term “genomic DNA” refers to any DNA sequence in a cell that constitutes the genetic make-up of the cell and is not limited to chromosomal DNA.

### 4. DETAILED DESCRIPTION OF THE INVENTION

The invention provides methods for the prevention and treatment of cancer and precancerous lesions in a subject, in which fusion cells are administered to the subject and wherein the fusion cells are formed by fusing antigen presenting cells, such as dendritic cells, and non-dendritic cells that contain genomic DNA extracted from a tumor cell or a pre-cancerous cell. A prophylactic or therapeutic amount of such fused cells is administered to a subject in need of such prevention or treatment. In certain embodiments, such fused cells are administered in combination with a therapeutically effective amount of a molecule which stimulates a humoral immune response and/or a cytotoxic T-lymphocyte response (CTL). In a preferred embodiment, the invention relates to methods comprising administration of a therapeutically effective amount of fusion cells in combination with a cytokine such as, but not limited to, IL-12.

According to the methods described herein, antigen presenting cells, such as dendritic cells, are fused to non-dendritic cells that contain genomic DNA extracted from a tumor cell or a pre-cancerous cell, wherein the non-dendritic cell contains an antigen characteristic of the cancer to be prevented or treated. Without being bound by theory, the genomic DNA extracted from a tumor cell or a pre-cancerous cell encodes an antigen or an epitope characteristic of the cancer to be treated. In other embodiments, the genomic DNA extracted from a tumor cell or a pre-cancerous cell causes the non-dendritic cell and upon fusion of the dendritic cell with the non-dendritic cell to express elevated levels of a protein whose levels are also elevated in the cancer or in the pre-cancerous lesion, respectively, that is to be treated or prevented. The resulting fusion cells comprising antigen presenting cells and non-dendritic cells that contain genomic DNA extracted from a tumor cell or from a pre-cancerous cell are used as a potent composition for the prevention of tumors comprising that antigen that is expressed by the fusion cells.

In certain embodiments of the invention, the fusion cells contain one or more molecules that display the antigenicity of the tumor or the pre-cancerous lesion. In certain embodiments of the invention, the fusion cells contain one or more antigens or epitopes of the tumor or the pre-cancerous lesion. In certain embodiments, the antigen is associated with the tumor or the pre-cancerous lesion. In certain embodiments, the antigen is expressed at at least 2-fold, at least 5-fold, at least 10-fold, at least 20-fold, at least 50-fold or at least 100-fold higher levels in the tumor or the pre-cancerous lesion than in any other tissue of the subject bearing the tumor or the pre-cancerous lesion. In certain embodiments, the antigen is expressed at at least 2-fold, at least 5-fold, at least 10-fold, at least 20-fold, at least 50-fold or at least 100-fold higher levels in the tumor or the pre-cancerous lesion than in the tissue or cell-type from which the tumor or the pre-cancerous lesion is derived. Examples of antigens that are associated with a particular tumor or cancer are listed in Section 4.8. Tumor-associated antigens or cancer-associated antigens include, but are not limited to, p53 and mutants thereof, Ras and mutants thereof, a Bcr/Ab1 breakpoint peptide, HER-2/neu, HPV E6, HPV E7, carcinoembryonic antigen, MUC-1, MAGE-1, MAGE-3, BAGE, GAGE-1, GAGE-2, N-acetylglucosaminyltransferase-V, p15, gp100, MART-1/MelanA, tyrosinase, TRP-1, beta.-catenin, MUM-1 and CDK4. Other tumor-associated tumor-antigens include KS ¼ pan-carcinoma antigen (Perez and Walker, 1990, J. Immunol. 142:3662-3667; Bumal, 1988, Hybridoma 7(4):407-415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63) and prostate specific membrane antigen. In certain, more specific, embodiments, the antigen or epitope that is common between the fusion cells and the tumor or the pre-cancerous lesion is specific to the tumor or the pre-cancerous lesion.

In one embodiment, this approach is advantageous when a specific antigen is not readily identifiable, as is generally the case with respect to pre-cancerous cells. For prevention of human cancer, for example, pre-cancerous cells are obtained directly from a pre-cancerous lesion of a patient, e.g. by biopsy. Subsequently, genomic DNA is extracted from the pre-cancerous cell and transfected or microinjected into non-dendritic cells. In this instance, fusion cells formed from such non-dendritic cells with antigen presenting cells, and compositions comprising such fusion cells, are highly specific for the cancer to be prevented.

In certain embodiments, the genomic DNA that has been extracted from the tumor cell or cell of a precancerous lesion is amplified before transfection or microinjection into non-dendritic cells. Amplification of the genomic DNA from the tumor cell or the precancerous lesion may be necessary if the amount of tissue obtained by biopsy is very small. In certain embodiments, the genomic DNA is amplified using Whole Genome Amplification (WGA). In more specific embodiments, the WGA is performed using Polymerase Chain Reaction with random oligonucleotides as primers. In certain embodiments, the genomic DNA is amplified using multiple displacement amplification (see, e.g., Dean et al., 2002, PNAS 99(8):5261-5266). In a specific embodiment, GenomiPhi™ (Amersham Biosciences) is used to amplify the genomic DNA.

Described below, are methods for the treatment and prevention of cancer and precancerous lesions. In particular, sections 4.1.1 and 4.1.2 describe the pre-cancerous cells and tumor cells that can be used as sources for the genomic DNA with the methods of the invention, non-dendritic cells that can be used for fusion with antigen presenting cells, antigen presenting cells that can be used for fusion with the non-dendritic cells, and fusion cells formed by fusion of non-dendritic cells that contain genomic DNA extracted from tumor cells or pre-cancerous cells with antigen presenting cells, that are used in the invention, as well as methods for the isolation, preparation, and/or generation of those cells. Target cancers that can be treated or prevented using such compositions are described below in Sections 4.12.

In certain embodiments, the antigen-presenting cells to be used for the generation of the fusion cells are autologous and the non-dendritic cells are autologous or heterologous. In certain embodiments, the non-dendritic cells to be used for the generation of the fusion cells are autologous and the antigen-presenting cells are autologous or heterologous. In certain embodiments, the non-dendritic cell or the dendritic cell or both are matched for MHC with the subject to be treated. In certain embodiments at least one MHC class I allele is common between the dendritic cell or the non-dendritic cell or both and the subject to be treated. In certain embodiments, the antigen presenting cell is a universal antigen presenting cell (see section 4.7).

The invention also provides methods for the prevention and treatment of cancer and precancerous lesions in a subject, in which fusion cells are administered to the subject, wherein the fusion cells are formed by fusing antigen presenting cells, such as dendritic cells, and non-dendritic cells that contain complementary DNA molecules (“cDNAs”) that have been synthesized from mRNA that has been extracted from a tumor cell or a pre-cancerous cell. Such fusion cells are administered to a subject in need of such prevention or treatment. In certain embodiments, such fusion cells are administered in combination with a molecule which stimulates a humoral immune response and/or a cytotoxic T-lymphocyte response (CTL).

The present invention also relates to the fusion cells that can be used with the methods of the invention. In certain embodiments, fusion cells of the invention are fusion cells formed by fusing antigen presenting cells, such as dendritic cells or universal antigen presenting cells (see section 4.7), and non-dendritic cells, wherein the non-dendritic cells comprise genomic DNA extracted from a cancer cell or a cell of a precancerous lesion; cDNA or a cDNA library derived from a cancer cell or a cell of a precancerous lesion; one or more expression constructs encoding a tumor-associated antigen; genomic DNA extracted from an infectious agent; genomic DNA extracted from a cell infected with an infectious agent; cDNA derived from an infectious agent; cDNA derived from a cell infected with an infectious agent; or one or more expression constructs encoding an antigen specific to an infectious agent. In certain embodiments, the fusion cells of the invention express one or more antigens of the cancer to be treated or prevented. In certain embodiments, the fusion cells of the invention express one or more antigens of the infectious agent to be treated or prevented.

In certain embodiments, a fusion cell of the invention is formed by fusion of a non-dendritic cell that contains genomic DNA or cDNA from a tumor cell and a universal antigen presenting cell. Such universal antigen presenting cells are described in section 4.7.

In certain embodiments of the invention, mRNA derived from a cancer cell, a cell of a precancerous lesion, or an infectious agent is introduced into a nondendritic cell before fusion of the non-dendritic cell to an antigen-presenting cell. Such fusion cells can then be used for the treatment and prevention of cancer or an infectious disease, respectively, as described above. In certain embodiments, cDNA that has been prepared from mRNA isolated from a cancer cell, a cell of a precancerous lesion, or an infectious agent can be used to transcribe mRNA, which is then introduced into the non-dendritic cell. In certain embodiments, mRNA encoding a tumor-associated antigen or an antigen specific to an infectious agent is introduced into the non-dendritic cell. Tumor-associated antigens are described in section 4.8.

In certain embodiments of the invention, mRNA derived from a cancer cell, a cell of a precancerous lesion, or an infectious agent is introduced into a universal antigen-presenting cell (see section 4.7). Such universal antigen-presenting cells can then be used for the treatment and prevention of cancer or an infectious disease, respectively, as described above for fusion cells. In certain embodiments, cDNA that has been prepared from mRNA isolated from a cancer cell, a cell of a precancerous lesion or an infectious agent can be used to transcribe mRNA, which is then introduced into a universal antigen-presenting cell. In certain embodiments, mRNA encoding a tumor-associated antigen or an antigen specific to an infectious agent is introduced into a universal antigen-presenting cell.

### 4.1 NON-DENDRITIC CELLS TRANSFORMED WITH GENOMIC DNA FROM A TUMOR CELL

For the fusion of non-dendritic cells that contain genomic DNA extracted from tumor cells or pre-cancerous cells with antigen presenting cells, different types of non-dendritic cells and non-dendritic cells from different sources can be used. The genomic DNA can be obtained from different sources by any method known to the skilled artisan. The genomic DNA can be transfected or microinjected into the non-dendritic cells by any method known to the skilled artisan.

A non-dendritic cell to be used with the methods of the invention for the generation of fusion cells have to be capable of being transformed or microinjected with genomic DNA and have to be capable of being fused with dendritic cells. Any method known to the skilled artisan can be used to determine whether a non-dendritic cell is suitable for the methods of the invention. Several criteria can be used to determine whether a non-dendritic cell is well-suited for use with the methods of the invention. In one aspect, a non-dendritic cell to be used with the methods of the invention should be capable of being transfected or microinjected with genomic DNA. In another aspect, a non-dendritic cell is capable of being fused with a dendritic cell. In another aspect, the ability of a non-dendritic cell to be used with the methods of the invention to grow in culture can also be a factor to be considered.

In certain embodiments, the non-dendritic cell is derived from a species different from the species of the subject that is to be treated. Alternatively, the non-dendritic cells are derived from the same species as the species of the subject that is to be treated. In certain embodiments, the non-dendritic cells are heterologous to the subject that is to be treated. In other embodiments, the non-dendritic cells are autologous to the subject that is to be treated. In certain embodiments, the non-dendritic cells are maintained and/or propagated in cell culture.

In certain embodiments, the non-dendritic cell is derived from a species different from the species from which the antigen presenting cells are derived. Alternatively, the non-dendritic cells are derived from the same species as the species from which the antigen presenting cells are derived. The non-dendritic cells may be from a primary cell culture that may be autologous, syngeneic, or allogeneic to the subject, depending on the source of the antigen presenting cells to be used in preparation of the fusion cells. In one embodiment, where the dendritic cell is allogeneic to the patient, the non-dendritic cell may have at least one MHC I allele that is of the same class I MHC haplotype as the mammal being treated. In another embodiment, where the dendritic cell is autologous to the patient, the non-dendritic cell may be an allogeneic or autologous to the mammal being treated.

In another embodiment, suitable non-dendritic cells are preferably isolated from the recipient or, less preferably, a family member or an individual who shares at least one MHC allele, and preferably the class I MHC haplotype, with the intended recipient and who carries the pre-cancerous lesions of the cancer to be prevented.

Where allogeneic antigen presenting cells are to be used, non-dendritic cells used for generation of fusion cells with allogeneic antigen presenting cells must have at least one common MHC allele in order to elicit an immune response in the mammal. Most preferred are non-dendritic cells derived from the intended recipient, i.e., the pre-cancerous non-dendritic cells are autologous to the patient to whom the fusion cells of the present invention are to be administered. In one embodiment, non-dendritic cells that are nonautologous, but share at least one MHC allele with the target pre-cancerous cells or cancer cells of the recipient may be used. If the non-dendritic cells are obtained from the same or from a syngeneic individual, such cells will have the same class I MHC haplotype. If they are not all obtained from the same subject or a syngeneic source, the MHC haplotype can be determined by standard HLA typing techniques well known in the art, such as serological tests and DNA analysis of the MHC loci. An MHC haplotype determination does not need to be undertaken prior to carrying out the procedure for generation of the fusion cells of the invention.

In a specific embodiment, the non-dendritic cells are fibroblasts. In even more specific embodiments, the non-dendritic cells are NIH 3T3 cells. In a specific embodiment, the non-dendritic cells are isolated. In even more specific embodiments, the non-dendritic cells are purified.

The genomic DNA for transfection or microinjection into the non-dendritic cells can be obtained from a cancer cell, a tumor cell or a precancerous lesion. In certain embodiments, the genomic DNA is obtained from the same type of tumor, cancer, or precancerous lesion as the tumor, cancer, or precancerous lesion to be treated in the subject. In certain embodiments, the genomic DNA is obtained from a cell of a tumor, cancer, or precancerous lesion that developed from the same tissue type as the tissue type form which the tumor, cancer, or precancerous lesion that is to be treated in the subject developed. In certain embodiments, the genomic DNA is obtained from a cultured tumor cell. In certain embodiments, the genomic DNA is extracted from a cell of a tumor, cancer, or precancerous lesion from a subject different from the subject to be treated. In a preferred embodiment, the genomic DNA is extracted from a cell of a tumor, cancer, or precancerous lesion from the subject to be treated. In a preferred embodiment, the genomic DNA is extracted from a cell of the tumor, cancer, or precancerous lesion to be treated in the subject.

In certain embodiments, the genomic DNA is extracted from a cell of a tumor, cancer, or precancerous lesion that has been obtained from a subject using biopsy. In a more specific embodiment, the cell of a tumor, cancer, or precancerous lesion has been obtained using a needle biopsy. In a more specific embodiment, the cell of a tumor, cancer, or precancerous lesion was obtained by biopsy from the subject that is to be treated.

Any method known to the skilled artisan can be used to extract genomic DNA from a cell of a tumor, cancer, or precancerous lesion. An illustrative method for isolating genomic DNA is described in Unit 2.2. of Short Protocols in Molecular Biology, Ausubel et al. (editors), John Wiley & Sons, Inc., 1999. In certain embodiment, the genomic DNA is treated very gently to avoid shearing of the DNA. In other embodiments, the genomic DNA is sheared to obtain smaller DNA fragments. In certain embodiments, the DNA is treated with DNAse-free protease to remove any proteinaceous substances from the DNA. In other embodiments, the genomic DNA is not treated with protease, instead care is taken to leave undisturbed the proteins associated with the genomic DNA. In certain embodiments, the DNA is treated with DNAse free RNAse.

The genomic DNA can be introduced into the non-dendritic cells using any method known to the skilled artisan. In certain embodiments, the genomic DNA is transfected into the non-dendritic cells. In more specific embodiments, the genomic DNA is transfected into the non-dendritic cells using lipofection. Illustrative methods for introducing the genomic DNA into non-dendritic cells are described in Chapter 9 of Short Protocols in Molecular Biology, Ausubel et al. (editors), John Wiley & Sons, Inc., 1999.

The optimal amount of genomic DNA to be introduced into the non-dendritic cells can be determined by standard techniques well-known to the skilled artisan. In certain embodiments, different populations of fusion cells are generated wherein the only difference between the different populations is the amount of genomic DNA that was introduced into the non-dendritic cells. The different populations of fusion cells are then tested for their effectiveness in preventing or treating a tumor. For a description of methods for testing the effectiveness of fusion cells of the invention in preventing or treating a tumor, see section 5. In other embodiments, the fusion cells are tested using an in vitro assay (see section 4.11). In certain embodiments, the amount of genomic DNA introduced per non-dendritic cell corresponds to at least the equivalent of 1 genome of a tumor cell or a precancerous cell, at least the equivalent of 10⁻¹ genome of a tumor cell or a precancerous cell, at least the equivalent of 10⁻² genome of a tumor cell or a precancerous cell, at least the equivalent of 10⁻³ genome of a tumor cell or a precancerous cell, at least the equivalent of 10⁻⁴ genome of a tumor cell or a precancerous cell, at least the equivalent of 10⁻⁵ genome of a tumor cell or a precancerous cell, at least the equivalent of 10⁻⁶ genome of a tumor cell or a precancerous cell, or at least the equivalent of 10⁻⁷ genome of a tumor cell or a precancerous cell. In certain embodiments, the amount of genomic DNA introduced per non-dendritic cell corresponds to at most the equivalent of 1 genome of a tumor cell or a precancerous cell, at most the equivalent of 10⁻¹ genome of a tumor cell or a precancerous cell, at most the equivalent of 10⁻² genome of a tumor cell or a precancerous cell, at most the equivalent of 10⁻³ genome of a tumor cell or a precancerous cell, at most the equivalent of 10⁻⁴ genome of a tumor cell or a precancerous cell, at most the equivalent of 10⁻⁵ genome of a tumor cell or a precancerous cell, at most the equivalent of 10⁻⁴ genome of a tumor cell or a precancerous cell, or at most the equivalent of 10⁻⁷ genome of a tumor cell or a precancerous cell.

Any method can be used to identify and isolate those non-dendritic cells in which the genomic DNA has been introduced. In certain embodiments, DNA that encodes a marker gene is introduced concurrently with the genomic DNA into the non-dendritic cells. Cells that are positive for the marker gene also harbor the genomic DNA. Any marker gene known to the skilled artisan can be used. Illustrative examples of marker genes include genes whose gene products confer resistancy to a particular antibiotic to the cells (e.g., neomycine resistancy), genes whose gene products enable a cell to grow on a medium that lacks a substance that is normally required by this cell for growth, or genes whose gene products encode a visual marker. A visual marker that can be used with the methods of the invention is, e.g., GFP. Cells in which the DNA encoding the visual marker and the genomic DNA have been introduced can be isolated using FACS.

In certain embodiments, the genomic DNA is introduced into the non-dendritic cells using microinjection.

In certain embodiments, fragments of the genomic DNA are packaged into vectors for propagation of the genomic DNA. Such vectors include, but are not limited to, bacteriophages, cosmids or YACs. Any method known to the skilled artisan can be used to package and propagate the genomic DNA.

Without being bound by theory, once the genomic DNA is introduced into a non-dendritic cell, the non-dendritic cell expresses one or more of the antigens that are expressed by the tumor cell, neoplastic cell or cell of a precancerous lesion from which the genomic DNA was isolated. In certain embodiments of the invention, the fusion cells contain one or more molecules that display the antigenicity of the tumor or the pre-cancerous lesion. In other embodiments, the antigen is associated with the tumor or the pre-cancerous lesion. In certain embodiments, the antigen is expressed at at least 2-fold, at least 5-fold, at least 10-fold, at least 20-fold, at least 50-fold or at least 100-fold higher levels in the tumor or the pre-cancerous lesion than in any other tissue of the subject bearing the tumor or the pre-cancerous lesion. In certain embodiments, the antigen is expressed at at least 2-fold, at least 5-fold, at least 10-fold, at least 20-fold, at least 50-fold or at least 100-fold higher levels in the tumor or the pre-cancerous lesion than in the tissue or cell-type from which the tumor or the pre-cancerous lesion is derived. In certain embodiments, the fusion cells and the tumor cell or precancerous cell share in common at least one epitope that is unique to the tumor cell or precancerous cell and is not present in any of the other tissues of the subject to be treated. Without being bound by theory, such an epitope is expressed in the precancerous cell or tumor cell due to a mutagenic event in the genome of the precancerous cell or the tumor cell. In certain, more specific, embodiments, the antigen or epitope that is common between the fusion cells and the tumor or the pre-cancerous lesion is specific to the tumor or the pre-cancerous lesion.

In certain embodiments, the genomic DNA is introduced into the non-dendritic cells together with a marker gene to facilitate the identification, enrichment, and/or isolation of cells that comprise the genomic DNA. In certain embodiments, the non-dendritic cells are co-transfected with the genomic DNA and a marker gene. In other embodiments, the non-dendritic cells are co-injected with genomic DNA and a marker gene. Depending on the nature of the marker gene, cells containing the genomic DNA and also the marker can be selected by growth in a medium that selects for the presence of that marker. If the marker confers bioluminescence on the cells, the transfected cells can be selected using FACS.

### 4.1.1 PRE-CANCEROUS CELLS

A pre-cancerous cell from which genomic DNA is isolated can be any pre-cancerous cell bearing at least one allele that distinguishes the pre-cancerous cell from a normal cell. Such pre-cancerous cells may be isolated from a variety of sources, such as, but not limited to, a pre-cancerous lesion of the patient in need of preventive treatment. Methods for isolation and preparation of such pre-cancerous cells are described in detail hereinbelow.

The source of the pre-cancerous cells is selected according to the cancer to be prevented. Preferably, the pre-cancerous cells are autologous to the patient being treated. Since the entire genomic DNA of the pre-cancerous cells are used in the present methods, it is not necessary to isolate, characterize, or even know the identities of, any antigens prior to performing the present methods. In a specific embodiment, the genomic DNA of a pre-cancerous cell encodes at least one antigen that is specific to the pre-cancerous cells.

In certain embodiments, the invention provides fusion cells that express at least one antigen expressed by a pre-cancerous cell as well as a cancer cell that develops therefrom, e.g., a tumor-specific antigen or a tumor-associated antigen, that is capable of eliciting an immune response against such pre-cancerous or cancer cells which develop therefrom. In one embodiment of the invention, cells isolated from pre-cancerous lesions, or pre-cancerous tissues are used to extract genomic DNA, which in turn is introduced into non-dendritic cells. Non-limiting examples of cancers that are amenable to the methods of the invention are listed in Section 4.12.

Pre-cancerous cells may be isolated by surgical excision or biopsy of any precancerous lesion, many of which are known in the art. In one embodiment, for example, pre-cancerous cells are isolated, by surgical excision or biopsy of a medically-recognized pre-cancerous lesion designated Barrett's metaplasia, which is a precursor of esophageal adenocarcinoma. This lesion is a heterologous lesion generally found in the region of the gastro-esophageal junction. Pre-cancerous cell clones isolated from such lesions exhibit genetic and biological heterogeneity including, for example, p53 mutations, p16 mutations, and aneuploidy. These alterations are accompanied by discrete changes in cellular proliferation, differentiation, and apoptosis, which underlie a evolution from normal cell through metaplasia—dysplasia—adenocarcinoma stages by which a pre-cancerous cell develops into a tumor cell. Similarly, intestinal metaplasia of the gastric cardia have been proposed as pre-cancerous lesions of adenocarcinoma of the gastric cardia (see, e.g., Jankowski et al., 1999, Molecular Evolution of the Metaplasia—dysplasia—adenocarcinoma Sequence in the Esophagus Am. J. Pathol. 154(4): 965-73; Jankowski et al., 2000, Barrett=s Metaplasia, Lancet 356(9247): 2079-85; Haringsma et al., 2001, Autofluorescence Endoscopy: Feasibility of Detection of GI Neoplasms Unapparent to White Light Endoscopy with an Evolving Technology, Gastrointest Endosc 53(6): 642-50; and Ruol et al., 2000, Intestinal Metaplasia is the Probable Common Precursor of Adenocarcinoma in Barrett Esophagus and Adenocarcinoma of the Gasric Cardia, Cancer 88(11): 2520-28).

In another embodiment, pre-cancerous cells are isolated by surgical excision or biopsy of gastrointestinal polyps which in many instances represent pre-cancerous lesions that progress, with time, to an adenocarcinoma. Methods for identification and excision of such polyps are well known in the art. Such polyps arise during the development of sporadic colorectal cancer as well as in the development and progression of the heritable diseases familial adenomatous polyposis (FAP), hereditary non-polyposis colorectal cancer (HNPCC), and juvenile polyposis (JPS) (see e.g. Souza, A, 2001, Molecular Rationale for the How, When, and Why of Colorectal Cancer Screening Ailment Pharmacol Ther 15(4): 451-62). FAP and HNPCC represent two well-defined forms of hereditary colorectal cancer: (a) familial adenomatous polyposis (FAP), which is caused by germ line mutations of adenomatous polyposis coli (APC) gene; and (b) hereditary nonpolyposis colorectal cancer (HNPCC), which is caused by germ line mutations of a mismatch repair gene (Boland C. R., Malignant tumors of the colon. In Textbook of Gastroenterology 2^{nd} Ed. (Eds. Yamada T) 1967-2026 (J. B. Lippincot Company, Philadelphia, (1995); Kinzler, et al., 1991, Identification of FAP locus genes from chromosome 5q21, Science 253:661-665; Lynch et al., 1996, Hereditary Nonpolyposis Colorectal Cancer (Lynch Syndrome); An updated review. Cancer 78:1149-1167; Peltomaki et al., 1997, Mutations predisposing to hereditary nonpolyposis colorectal Cancer: database and results of a collaborative study, Gastroenterology 113:1146-1158). These hereditary colorectal cancers are characterized by their early onset and high mortality rate. In all FAP patients, adenomatous polyps develop at a median patient age of 16 years, and virtually all affected individuals develop cancer by a median age of 39 years (Boland C. R., Malignant tumors of the colon, in Textbook of Gastroenterology 2^{nd} Ed. (Eds. Yamada T) 1967-2026 (J. B. Lippincot Company, Philadelphia, (1995)). Mutation of APC gene is also observed in 70-80% of sporadic colon cancer patients (Nakamura Y., 1997, Cleaning up on β-catenin. News & Views. Nature Medicine 3, 499-500).

In still another embodiment, pre-cancerous cells are isolated by surgical excision or biopsy of intratubular epithelial dysplasia, which is the most common medically-recognized precursor of renal cell carcinoma. In another aspect of this embodiment, pre-cancerous cells are isolated, by surgical excision or biopsy of one or more of the well-documented pre-cancerous lesions of the vonHippel-Lindau syndrome. In this disease, there is an evolution from a pre-cancerous, simple cyst, through an atypical cyst with epithelial hyperplasia, and culminating in a cystic or solid renal cell carcinoma. Moreover, a developmental sequence progressing from pre-cancerous adenomatous lesions to carcinomas has also been observed in papillary renal cell carcinoma. Accordingly, such pre-cancerous adenomatous lesions are also useful sources for isolation of pre-cancerous non-dendritic cells (see e.g. VanPoppel et al. Precancerous Lesions in the Kidney, Scand. J. Urol. Nephrol. Suppl. 205: 136-65 (2000)).

In another embodiment, pre-cancerous cells are isolated, by surgical excision or, preferably by biopsy of dysplasia detected during screening endoscopic retrograde cholangiopancreatography (ERCP) procedures. ERCP screening is indicted in instances of familial pancreatic cancer, and in instances of hereditary pancreatitis, which is associated with a 40% lifetime risk of developing pancreatic ductal adenocarcinoma (see e.g. Howes et al. Screening for Early Pancreatic Ductal Adenocarcinoma in Hereditary Pancreatitis, Med. Clin. North Am. 84(3): 719-38 (2000); and Brentnall, Cancer Surveillance of Patients from Familial Pancreatic Cancer Kindreds Med. Clin. North Am. 84(3): 707-18 (2000)).

In still another embodiment, pre-cancerous cells are isolated by surgical excision or by biopsy of actinic keratoses, benign nevi, and dysplasic nevi. Actinic keratoses and pre-cancerous lesions characteristic of Bowen=s disease (squamous cell carcinoma in situ) provide non-cancerous cells that are precursors to the development of squamous cell carcinoma (SSC), while benign nevi, and dysplasic nevi are potential precursors of malignant melanoma (see e.g. Gloster et al. The Epidemiology of Skin Cancer, Dermatol. Surg. 22(3): 217-26 (1996); and Sober et al. Precursors to Skin Cancer, Cancer 75(2 Suppl.): 645-50 (1995)).

In another embodiment, pre-cancerous cells are isolated by surgical excision or biopsy of pre-cancerous lesions leading to breast cancer. It has been reported that atypical cystic duct (ACD) is the precancerous lesion of breast cancer based upon an observed histologic continuum between ACD and malignancy and because of the expression of the p53 protein in ACD (Kusama et. al. Clinicopathological Characteristics of Atypical Cystic Duct (ACD) of the Breast: Assessment of ACD as a Precancerous Lesion, Pathol. Int. 50(10): 793-800 (2000)). Similarly, noncomedo ductal carcinoma in situ (DCIS) lesions and especially comedo ductal carcinoma in situ lesions are associated with an elevated risk (more than eight-fold) of developing invasive breast cancer, and, therefore are sources for isolation of pre-cancerous non-dendritic cells useful in the present invention (see, e.g., Lawrence et al. A High-Rish Lesion for Invasive Breast Cancer, Ductal Carcinoma in Situ, Exhibits Frequent Overexpression of Retinoid X Receptor, Cancer Epidemiol. Biomarkers Prev. 7(1): 29-35 (1998)).

In a further embodiment, pre-cancerous cells are isolated, by surgical excision or biopsy of high-grade prostatic intraepithelial neoplasia lesions, which are recognized pre-cancerous lesions important in neoplastic development, especially when accompanied by adjacent atypical glands (Sakr et al. Histological Markers of Risk and the Role of High-Grade Prostatic Intraepithelial Neoplasia, Urology 57(4): 115-20 (2001); Zlotta et al. Clinical Evolution of Prostatic Intraepithelial Neoplasia, Eur. Urol. 35(5-6): 498-503 (1999); Alsikafi et al. High-Grade Prostatic Intraepithelial Neoplasia with Adjacent Atypia is Associated with a Higher Incidence of Cancer on Subsequent Needle Biopsy Than High-Grade Prostatic Intraepithelial Neoplasia Alone, Urology 57(2): 296-300 (2001); and Molinie, Prostatic Intraepithelial Neoplasia, Ann. Pathol. 21(3): 245-254 (2001)).

In a still further embodiment, pre-cancerous cells are isolated by surgical excision or biopsy of any one of at least three different lesions that are regarded as comprising pre-cancerous cells of lung cancer: (1) squamous dysplasia and carcinoma in situ; (2) atypical adenomatous hyperplasia; and (3) diffuse idiopathic pulmonary neuroendocrine cell hyperplasia (Kerr, Pulmonary Preinvasive Neoplasia, J. Clin. Pathol. 54(4): 257-71 (2001)).

In another embodiment, pre-cancerous cells are isolated by surgical excision or biopsy of oral leukoplakia, which can appear as a white patch on oral mucosa, that are recognized as pre-cancerous lesions which have a high probability of developing into oral cancer (Mao, Leukoplakia: Molecular Understanding of Pre-malignant Lesions and Implications for Clinical Management, Mol. Med. Today, 3(10): 442-48 (1997)).

Although specific sources of pre-cancerous cells have been disclosed above with respect to colorectal, prostatic, esophageal, renal, pancreatic, skin, breast, lung, and oral cancers, the present invention is not to be limited to these specific embodiments. That is, as is apparent to one of ordinary skill, pre-cancerous tissues are readily characterized as hyperplasic, metaplasic, and dysplasic, and which comprise pre-cancerous cells having at least one genetic allele that distinguishes those pre-cancerous cells from normal cells. In addition, genetic tests, which are now available and will be developed as analysis of the human genome continues, that permit rapid and precise identification of the presence of specific alleles associated with an increased risk of cancer development. Accordingly, identification and analysis of pre-cancerous tissues suitable for use as sources of pre-cancerous non-dendritic cells of the present invention are readily performed by, inter alia, oncologists and, more particularly, molecular oncologists of ordinary skill.

In certain embodiments, the pre-cancerous cells are not freshly isolated, but are instead cultured to select for pre-cancerous cells to isolate genomic DNA from for introduction into the non-dendritic cells, which are to be fused with antigen presenting cells and thereby prevent or limit contamination of a population of pre-cancerous cells with healthy, non-precancerous cells.

In a preferred embodiment, the pre-cancerous cells of the invention are isolated from a pre-cancerous lesion that is surgically removed from the mammal that will be the recipient of the fusion-cell containing compositions. Prior to use, solid pre-cancerous tissue or aggregated pre-cancerous cells can be dispersed, preferably mechanically, into a single cell suspension by standard techniques. Enzymes, such as but not limited to, collagenase and DNase may also be used to disperse cancer cells. In certain embodiments, however, genomic DNA is isolated from the pre-cancerous tissue without prior dispersion of the cells. If the pre-cancerous cells are to be cultured, prior dispersion of the cells is the preferred embodiment. In yet another preferred embodiment, the pre-cancerous cells of the invention are obtained from primary cell cultures, ie., cultures of original cells obtained from the body.

The amount of pre-cancerous cells collected should be sufficient to isolate enough genomic DNA to generate enough non-dendritic cells comprising the genomic DNA to fuse those non-dendritic cells with antigen presenting cells to prepare enough fusion cells for the vaccines of the invention.

In another embodiment, suitable pre-cancerous cells are preferably of the same cell type as the cancer desired to be inhibited and are isolated from the recipient or, less preferably, a family member or an individual who shares at least one MHC allele, and preferably the class I MHC haplotype, with the intended recipient and who carries the pre-cancerous lesions of the cancer to be prevented.

Pre-cancerous cells, such as cells containing an antigen having the antigenicity of a cancer cell can be identified and isolated by any method known in the art. For example, pre-cancerous cells can be identified by morphology, enzyme assays, proliferation assays, or the presence of cancer-causing viruses. If the characteristics of the antigen of interest are known, pre-cancerous cells can also be identified or isolated by any biochemical or immunological methods known in the art. For example, pre-cancerous cells can be isolated by surgery, endoscopy, other biopsy techniques, affinity chromatography, and fluorescence activated cell sorting (e.g., with fluorescently tagged antibody against an antigen expressed by the pre-cancerous non-dendritic cells).

There is no requirement that a clonal or homogeneous or purified population of pre-cancerous non-dendritic cells be used. A mixture of cells can be used, provided that a substantial number of cells in the mixture contain the antigen or antigens of the pre-cancerous cells being targeted. In a specific embodiment, the pre-cancerous cells and/or antigen presenting cells are purified.

Without being bound by theory, a mutagenic event in the precancerous cell results in the expression of an antigen by the pre-cancerous cell that is unique to the precancerous cell.

### 4.1.2 TUMOR CELLS

A tumor cell from which genomic DNA is isolated can be any tumor cell bearing at least one allele that distinguishes the tumor cell from a normal cell. Tumor cells may be isolated from a variety of sources, such as, but not limited to, a tumor of the patient in need of preventive treatment. Methods for isolation and preparation of such tumor cells are described in detail hereinbelow.

Without being bound by theory, cancerous or tumor tissue is characterized by one or more of the following: self-sufficiency in growth signals; insensitivity to anti-growth signals; tissue invasion and metastasis; sustained angiogenesis; and evading apoptosis. A more detailed description of cancer can be found, e.g., in Hanahan and Weinberg, 2000, Cell 100:57-70, which is incorporated herein in its entirety.

The source of the tumor cells is selected according to the tumor to be treated or prevented. Preferably, the tumor cells are autologous to the patient being treated. Since the entire genomic DNA of the tumor cells are used in the present methods, it is not necessary to isolate, characterize, or even know the identities of, any antigens prior to performing the present methods. In a specific embodiment, the genomic DNA of a tumor cell encodes at least one antigen that is specific to the tumor cell.

In certain embodiments, the invention provides fusion cells that express at least one antigen expressed by a tumor cell, e.g., a tumor-specific antigen or a tumor associated antigen, that is capable of eliciting an immune response against such tumor cell. In one embodiment of the invention, cells isolated from tumor tissue are used to extract genomic DNA, which in turn is introduced into non-dendritic cells. Non-limiting examples of cancers that are amenable to the methods of the invention are listed in Section 4.12.

Tumor cells may be isolated by surgical excision or biopsy of any precancerous lesion, many of which are known in the art. In certain embodiments, the tumor cells are not freshly isolated, but are instead cultured to select for tumor cells to isolate genomic DNA from for introduction into the non-dendritic cells, which are to be fused with antigen presenting cells and thereby prevent or limit contamination of a population of pre-cancerous cells with healthy, non-precancerous cells.

In a preferred embodiment, the tumor cells of the invention are isolated from a tumor that is surgically removed from the mammal that will be the recipient of the fusion-cell containing compositions. Prior to use, solid tumor tissue can be dispersed, preferably mechanically, into a single cell suspension by standard techniques. Enzymes, such as, but not limited to, collagenase and DNase may also be used to disperse cancer cells. In certain embodiments, however, genomic DNA is isolated from the tumor tissue without prior dispersion of the cells. If the tumor cells are to be cultured, prior dispersion of the cells is the preferred embodiment. In yet another preferred embodiment, the tumor cells for use with the methods of the invention are obtained from primary cell cultures, ie., cultures of original cells obtained from the body.

In certain embodiments, the amount of tumor cells collected is sufficient to isolate enough genomic DNA to generate enough non-dendritic cells comprising the genomic DNA to fuse those non-dendritic cells with antigen presenting cells to prepare enough fusion cells for the vaccines of the invention. If not enough genomic DNA can be isolated to generate enough fusion cells for treatment or prevention, the genomic DNA can be amplified by any technique known to the skilled artisan. In a certain, more specific embodiments, the genomic DNA is amplified by Whole Genome Amplification.

In another embodiment, suitable tumor cells are preferably of the same cell type as the cancer desired to be inhibited and are isolated from the recipient or, less preferably, a family member or an individual who shares at least one MHC allele, and preferably the class I MHC haplotype, with the intended recipient and who carries the pre-cancerous lesions of the cancer to be prevented.

Tumor cells, such as cells containing an antigen having the antigenicity of a cancer cell can be identified and isolated by any method known in the art. For example, tumor cells can be identified by morphology, enzyme assays, proliferation assays, or the presence of cancer-causing viruses. If the characteristics of the antigen of interest are known, tumor cells can also be identified or isolated by any biochemical or immunological methods known in the art. For example, tumor cells can be isolated by surgery, endoscopy, other biopsy techniques, affinity chromatography, and fluorescence activated cell sorting (e.g., with fluorescently tagged antibody against an antigen expressed by the pre-cancerous non-dendritic cells).

There is no requirement that a clonal or homogeneous or purified population of pre-cancerous non-dendritic cells be used. A mixture of cells can be used, provided that a substantial number of cells in the mixture contain the antigen or antigens of the tumor cells being targeted. In a specific embodiment, the tumor cells and/or antigen presenting cells are purified.

Without being bound by theory, a mutagenic event in the precancerous cell results in the expression of an antigen by the pre-cancerous cell that is unique to the tumor cell.

Without being bound by theory, a mutagenic event in the tumor cell or the cancer cell results in the expression of an antigen by the tumor cell or the cancer cell that is unique to the tumor cell or the cancer cell.

### 4.2 NON-DENDRITIC CELLS COMPRISING DNA FROM AN INFECTIOUS AGENT

In certain embodiments of the invention, fusion cells are generated to treat and/or prevent an infectious disease. Such fusion cells are generated by fusing antigen presenting cells with non-dendritic cells, wherein DNA of an infectious agent has been introduced into the non-dendritic cell. In certain embodiments, the DNA of an infectious agent is extracted directly from the infectious agent. In other embodiments, the DNA to be introduced into the non-dendritic cells is extracted from a cell that is infected with the infectious agent.

The non-dendritic cells may be from a primary cell culture that may be autologous, syngeneic, or allogeneic to the subject, depending on the source of the antigen presenting cells to be used in preparation of the fusion cells. In one embodiment, where the dendritic cell is allogeneic to the patient, the non-dendritic cell may have at least one MHC I allele that is of the same class I MHC haplotype as the mammal being treated. In another embodiment, where the dendritic cell is autologous to the patient, the non-dendritic cell may be an allogeneic or autologous to the mammal being treated.

In another embodiment, suitable non-dendritic cells are preferably isolated from the recipient or, less preferably, a family member or an individual who shares at least one MHC allele, and preferably the class I MHC haplotype, with the intended recipient and who is infected with the infectious agent or who is at risk of being infected with the infectious agent.

Where allogeneic antigen presenting cells are to be used, non-dendritic cells used for generation of fusion cells with allogeneic antigen presenting cells must have at least one common MHC allele in order to elicit an immune response in the mammal. Most preferred are non-dendritic cells derived from the intended recipient, ie., the non-dendritic cells are autologous to the patient to whom the fusion cells of the present invention are to be administered. In one embodiment, non-dendritic cells that are nonautologous, but share at least one MHC allele with the target pre-cancerous cells or cancer cells of the recipient may be used. If the non-dendritic cells are obtained from the same or from a syngeneic individual, such cells will have the same class I MHC haplotype. If they are not all obtained from the same subject or a syngeneic source, the MHC haplotype can be determined by standard HLA typing techniques well known in the art, such as serological tests and DNA analysis of the MHC loci. An MHC haplotype determination does not need to be undertaken prior to carrying out the procedure for generation of the fusion cells of the invention.

In a specific embodiment, the non-dendritic cells are fibroblasts. In even more specific embodiments, the non-dendritic cells are NIH 3T3 cells. In a specific embodiment, the non-dendritic cells are isolated. In even more specific embodiments, the non-dendritic cells are purified.

The infected cell from which the DNA is isolated can be an infected cell obtained from the subject that is to be treated. In other embodiments, the cell from which the DNA is isolated is obtained from a first subject different from the subject to be treated, the second subject, wherein the first subject is infected with or has been exposed to the infectious agent that is to be treated or prevented in the second subject. In other embodiments, the DNA is obtained from an infected cell, wherein the infected cell is maintained and propagated in vitro. Target infectious diseases and illustrative infectious diseases are described in section 4.2.1.

Any method known to the skilled artisan can be used to extract genomic DNA, introduce the genomic DNA into non-dendritic cells, to fuse the non-dendritic cells with antigen presenting cells, to maintain the fusion cells, to inactivate the fusion cells and to administer the fusion cells. In particular the same methods that can be used to generate and use the fusion cells for treatment of cancer can also be used for the fusion cells for the treatment and prevention of infectious diseases. If the genomic DNA is derived from a cell that is infected with an infectious agent, the amount of genomic DNA introduced per non-dendritic cell corresponds to at least the equivalent of 1 genome of the infected cell, at least the equivalent of 10⁻¹ genome of the infected cell, at least the equivalent of 10⁻² genome of the infected cell, at least the equivalent of 10⁻³ genome of the infected cell, at least the equivalent of 10⁻⁴ genome of the infected cell, at least the equivalent of 10⁻⁵ genome of the infected cell, at least the equivalent of 10⁻⁶ genome of the infected cell, or at least the equivalent of 10⁻⁷ genome of the infected cell. In certain embodiments, the amount of genomic DNA introduced per non-dendritic cell corresponds to at most the equivalent of 1 genome of the infected cell, at most the equivalent of 10⁻¹ genome of the infected cell, at most the equivalent of 10⁻² genome of the infected cell, at most the equivalent of 10⁻³ genome of the infected cell, at most the equivalent of 10⁻⁴ genome of the infected cell, at most the equivalent of 10⁻⁵ genome of the infected cell, at most the equivalent of 10⁻⁶ genome of the infected cell, or at most the equivalent of 10⁻⁷ genome of the infected cell.

In a specific embodiment, if the infectious agent is a virus whose genome is partially or entirely integrated into the genome of the host, the genomic DNA to be introduced into the nondendritic cells is the genomic DNA of the host cell into whose genome the viral genome is integrated.

In certain embodiments, the infecious agent is an RNA virus, ie., the genome of the virus is composed of RNA. In such a case, genomic RNA is used with the methods of the invention or cDNA is prepared that encodes the genomic RNA of the RNA virus and the cDNA is used with the methods of the invention.

### 4.2.1 TARGET INFECTIOUS DISEASES

Infectious diseases that can be treated or prevented by the methods of the present invention are caused by infectious agents including, but not limited to, viruses, bacteria, fungi protozoa, helminths, and parasites. Combination therapy encompasses in addition to the administration of pharmaceutical compositions of the invention, the uses of one or more modalities that aid in the prevention or treatment of infectious diseases, which modalities include, but is not limited to antibiotics, antivirals, antiprotozoal compounds, antifungal compounds, and antihelminthics. Other treatment modalities that can be used to treat or prevent infectious diseases include immunotherapeutics, polynucleotides, antibodies, cytokines, and hormones as described above.

Infectious virus of both human and non-human vertebrates, include retroviruses, RNA viruses and DNA viruses. Examples of virus that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picomaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B, virus), the agents of non-A, non-B hepatitis (class 1=internally transmitted; class 2=parenterally transmitted (i.e. Hepatitis C); Norwalk and related viruses, and astroviruses).

Retroviruses that are contemplated include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picomaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Many examples of antiviral compounds that can be used in combination with the complexes of the invention are known in the art and include but are not limited to: rifampicin, nucleoside reverse transcriptase inhibitors (e.g., AZT, ddI, ddC, 3TC, d4T), non-nucleoside reverse transcriptase inhibitors (e.g., Efavirenz, Nevirapine), protease inhibitors (e.g., aprenavir, indinavir, ritonavir, and saquinavir), idoxuridine, cidofovir, acyclovir, ganciclovir, zanamivir, amantadine, and Palivizumab. Other examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime.

Bacterial infections or diseases that can be treated or prevented by the methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include but are not limited to infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Antibacterial agents or antibiotics that can be used in combination with the complexes of the invention include but are not limited to: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Additional examples of antibacterial agents include but are not limited to Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambenmycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmnenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Niftiratrone; Nifurdazil; Nifurimide; Nifirpirinol; Nifurquinazol; Nifurthiazole; Nitrocycline; Nitroftirantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivamnpicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafingin; Sisomicin; Sisomicin Sulfate; Sparfloxacin; Spectinomycin Hydrochloride; Spiramycin; Stallimycin Hydrochloride; Steffinycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Fungal diseases that can be treated or prevented by the methods of the present invention include but not limited to aspergilliosis, crytococcosis, sporotrichosis, coccidioidomycosis, paracoccidioidomycosis, histoplasmosis, blastomycosis, zygomycosis, and candidiasis.

Antifungal compounds that can be used in combination with the complexes of the invention include but are not limited to: polyenes (e.g., amphotericin b, candicidin, mepartricin, natamycin, and nystatin), allylamines (e.g., butenafme, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochloride.

Parasitic diseases that can be treated or prevented by the methods of the present invention including, but not limited to, amebiasis, malaria, leishmania, coccidia, giardiasis, cryptosporidiosis, toxoplasmosis, and trypanosomiasis. Also encompassed are infections by various worms, such as but not limited to ascariasis, ancylostomiasis, trichuriasis, strongyloidiasis, toxoccariasis, trichinosis, onchocerciasis. filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

### 4.3 GENOMIC DNA

Genomic DNA can be obtained from a tumor cell, a precancerous cell, a cell infected with an infectious agent or an infectious agent by any method known to the skilled artisan. Exemplary methods for the preparation of genomic DNA from mammalian tissue are described in Unit 2.2 in Short Protocols in Molecular Biology, 4^{th} edition, Ausubel et al. (editors), John Wiley & Sons, Inc., 1999. In certain embodiments, the genomic DNA that has been extracted from the tumor cell, the cell of a precancerous lesion, the cell infected with an infectious agent or the infectious agent is amplified before transfection or microinjection into non-dendritic cells. Amplification of the genomic DNA from the tumor cell or the precancerous lesion may be necessary if the amount of tissue obtained by biopsy is very small. In certain embodiments, the genomic DNA is amplified using Whole Genome Amplification (WGA). In more specific embodiments, the WGA is performed using Polymerase Chain Reaction with random oligonucleotides as primers. In certain embodiments, the genomic DNA is amplified using multiple displacement amplification (see, e.g., Dean et al., 2002, PNAS 99(8):5261-5266). In a specific embodiment, GenomiPhi™ (Amersham Biosciences) is used to amplify the genomic DNA. Any other method known to the skilled artisan may be used to amplify the genomic DNA.

In certain embodiments, the genomic DNA is treated with RNase to remove any RNA molecules. In certain embodiments, the genomic DNA is treated with protease to remove any proteinaceous material that may be associated with the genomic DNA. In certain embodiments, the genomic DNA is fractionated into fractions of DNA fragments of certain sizes. In certain embodiments, the average size of the genomic DNA fragments is at least 0.1 kb, 0.25 kb, 0.5 kb, 1 kb, 2 kb, 5 kb, 10 kb, 15 kb, 25 kb, 50 kb or at least 100 kb. In certain embodiments, the average size of the genomic DNA fragments is at most 0.1 kb, 0.25 kb, 0.5 kb, 1 kb, 2 kb, 5 kb, 10 kb, 15 kb, 25 kb, 50 kb or at most 100 kb. In certain embodiments, the genomic DNA fragments are between 0.1 kb and 0.5 kb, between 0.1 kb and 1 kb, between 0.1 kb and 2.5 kb, between 0.1 kb and 5 kb, between 0.1 kb and 10 kb, between 0.1 kb and 25 kb, between 0.1 kb and 50 kb, between 0.1 kb and 100 kb, between 0.5 kb and 1 kb, between 0.5 kb and 2.5 kb, between 0.5 kb and 5 kb, between 0.5 kb and 10 kb, between 0.5 kb and 25 kb, between 0.5 kb and 50 kb, between 0.5 kb and 100 kb, between 1 kb and 2.5 kb, between 1 kb and 5 kb, between 1 kb and 10 kb, between 1 kb and 25 kb, between 1 kb and 50 kb, between 1 kb and 100 kb, between 2.5 kb and 5 kb, between 2.5 kb and 10 kb, between 2.5 kb and 25 kb, between 2.5 kb and 50 kb, between 2.5 kb and 100 kb, between 5 kb and 10 kb, between 5 kb and 25 kb, between 5 kb and 50 kb, between 5 kb and 100 kb, between 10 kb and 25 kb, between 10 kb and 50 kb, between 10 kb and 100 kb, between 25 kb and 50 kb, between 25 kb and 100 kb, or between 50 kb and 100 kb. Any method known to the skilled artisan can be used to fractionate the genomic DNA. In certain illustrative embodiments, the genomic DNA is fractionated by shearing forces, e.g., by passing the DNA through a syringe. In another illustrative embodiment, the DNA is fractionated by sonication.

In certain embodiments, the genomic DNA fragments are small enough to be efficiently tranformed into a cell, yet large enough to contain at least one average sized open reading frame.

In certain embodiments, laser capture microdissection is used to obtain tumor cells or cells of a precancerous lesions. In an exemplary embodiment, AutoPix™ Automated Laser Capture Microdissection (LCM) System (Arcturus, Calif.) can be used to isolate tumor cells or cells of a precancerous lesion from a tissue sample.

Genomic DNA can subsequently be prepared from the cells that have been obtained by laser capture microdissection. The genomic DNA can be amplified by any method known to the skilled artisan. In a specific embodiment, tissue is obtained by biopsy from a subject, the tissue is fixed and subsequently subjected to laser capture microdissection to obtain tumor cells or cells of a precancerous lesion from the tissue. In certain embodiments, tumor cells or precancerous lesions are selected based on their morphology. In other embodiments, tumor cells or precancerous lesions are distinguished from the surrounding tissue using markers that are specific to the tumor or the precancerous lesion.

### 4.4 FUSION OF ANTIGEN-PRESENTING CELLS WITH NON-DENDRITIC CELLS THAT CONTAIN cDNA DERIVED FROM A TUMOR OR PRE-CANCEROUS CELL OR INFECTIOUS AGENT

The invention also provides methods for the prevention and treatment of cancer and precancerous lesions in a subject, in which fusion cells are administered to the subject and wherein the fusion cells are formed by fusing antigen presenting cells, such as dendritic cells, and non-dendritic cells that contain complementary DNA molecules (“cDNAs”) that have been synthesized from mRNA that has been extracted from a tumor cell or a pre-cancerous cell. A prophylactic or therapeutic amount of such fused cells is administered to a subject in need of such prevention or treatment. In certain embodiments, such fused cells are administered in combination with a therapeutically effective amount of a molecule which stimulates a humoral immune response and/or a cytotoxic T-lymphocyte response (CTL). In a preferred embodiment, the invention relates to methods comprising administration of a therapeutically effective amount of fusion cells in combination with a cytokine such as, but not limited to, IL-12.

According to the methods described herein, antigen presenting cells, such as dendritic cells, are fused to non-dendritic cells that contain cDNAs that have been synthesized from mRNA that has been extracted from a tumor cell or a pre-cancerous cell, wherein the non-dendritic cell contains an antigen or epitope characteristic of the cancer to be prevented or treated. Without being bound by theory, one or more of the cDNAs that were synthesized from mRNA of the tumor cell or precancerous cell encodes an antigen or epitope characteristic of the cancer to be prevented or treated. In other embodiments, one or more of the cDNAs that were synthesized from mRNA of the tumor cell or precancerous cell causes the non-dendritic cell (and upon fusion of the dendritic cell with the non-dendritic cell, the fusion cell) to express elevated levels of a protein whose levels are also elevated in the cancer or in the pre-cancerous lesion, respectively, that is to be treated or prevented. Elevated levels of a protein refer to levels of the protein in the cancer cell or the precancerous cell relative to a non-cancer cell. The resulting fusion cells comprising antigen presenting cells and non-dendritic cells that contain cDNA derived from a tumor cell or from a pre-cancerous cell are used as a potent composition for the prevention of tumors comprising that antigen that is expressed by the fusion cells.

In certain embodiments of the invention, the fusion cells contain one or more molecules that display the antigenicity of the tumor or the pre-cancerous lesion. In certain embodiments of the invention, the fusion cells contain one or more antigens or epitopes of the tumor or the pre-cancerous lesion. In other embodiments, the antigen is associated with the tumor or the pre-cancerous lesion. In certain embodiments, the antigen is expressed at at least 2-fold, at least 5-fold, at least 10-fold, at least 20-fold, at least 50-fold or at least 100-fold higher levels in the tumor or the pre-cancerous lesion than in any other tissue of the subject bearing the tumor or the pre-cancerous lesion. In certain embodiments, the antigen is expressed at at least 2-fold, at least 5-fold, at least 10-fold, at least 20-fold, at least 50-fold or at least 100-fold higher levels in the tumor or the pre-cancerous lesion than in the tissue or cell-type from which the tumor or the pre-cancerous lesion is derived. In certain, more specific, embodiments, the antigen or epitope that is common between the fusion cells and the tumor or the pre-cancerous lesion is specific to the tumor or the pre-cancerous lesion.

In certain embodiments of the invention at least 10⁻⁸ g of cDNA are introduced per non-dendritic cell or a universal antigen presenting cell. In certain embodiments of the invention at least 10⁻⁹ g of cDNA are introduced per non-dendritic cell or a universal antigen presenting cell. In certain embodiments of the invention at least 10⁻¹⁰ g of cDNA are introduced per non-dendritic cell or a universal antigen presenting cell. In certain embodiments of the invention at least 10⁻¹¹ g of cDNA are introduced per non-dendritic cell or a universal antigen presenting cell. In certain embodiments of the invention at least 10⁻¹² g of cDNA are introduced per non-dendritic cell or a universal antigen presenting cell.

In certain embodiments of the invention at most 10⁻⁸ g of cDNA are introduced per non-dendritic cell or a universal antigen presenting cell. In certain embodiments of the invention at most 10⁻⁹ g of cDNA are introduced per non-dendritic cell or a universal antigen presenting cell. In certain embodiments of the invention at most 10⁻¹⁰ g of cDNA are introduced per non-dendritic cell or a universal antigen presenting cell. In certain embodiments of the invention at most 10⁻¹¹ g of cDNA are introduced per non-dendritic cell or a universal antigen presenting cell. In certain embodiments of the invention at most 10⁻¹² g of cDNA are introduced per non-dendritic cell or a universal antigen presenting cell.

In one embodiment, this approach is advantageous when a specific antigen is not readily identifiable, as is generally the case with respect to pre-cancerous cells. For prevention of human cancer, for example, pre-cancerous cells are obtained directly from a pre-cancerous lesion of a patient, e.g. by biopsy. Subsequently, mRNA is extracted from the pre-cancerous cell and cDNA molecules are synthesized from the mRNA. In certain embodiments, the cDNAs are subsequently amplified. In certain embodiments, the cDNAs are enriched for tumor-specific or tumor-associated cDNAs. In certain embodiments, a cDNA library is generated from the cDNAs that have been derived from the tumor cell or the precancerous cell. The cDNAs, which may in certain embodiments be amplified or enriched for tumor-specific or tumor-associated cDNAs or in the form of a cDNA library, are then transfected or microinjected into non-dendritic cells. In this instance, fusion cells formed from such non-dendritic cells with antigen presenting cells, and compositions comprising such fusion cells, are highly specific for the cancer to be prevented.

mRNA can be obtained from the cancerous cell or precancerous cell by any method known to the skilled artisan. In an exemplary embodiment, total RNA is first obtained from a cancerous cell or precancerous cell using any one of the many commercially available kits, e.g., from Ambion, Inc. Subsequently, poly(A) RNA can be isolated from the total RNA, e.g., using an oligo-dT column. In another embodiment, poly(A) RNA is directly isolated from the tissue, e.g., by using using any one of the many commercially available kits, e.g., from Ambion, Inc. The poly(A) RNA can then be used as a template for cDNA synthesis using reverse transcription. Reverse transcription results in a single stranded cDNA. The second strand can then be synthesized using any method known to the skilled artisan. In a specific embodiment, the second strand is synthesized using the Klenow fragment of E. coli DNA polymerase I. The primer for the polymerase reaction is provided by the hairpin loop that forms from the complementary tail at the 5′ end of the cDNA produced by the reverse transcription. In another embodiment, RNase H, E. coli DNA polymerase I and DNA ligase are used to synthesize the second strand of the cDNA. The cDNA can then be amplified using PCR or the cDNA molecules can be ligated into a cloning vector to create a cDNA library. In certain embodiments, oligonucleotides of known sequence can be ligated at the cDNA and oligonucleotides complementary to those sequences are used as primers for the PCR. Amplification of cDNAs from the tumor cell or the precancerous lesion may be necessary if the amount of tissue obtained by biopsy is very small. cDNAs may be amplified by any method known to the skilled artisan.

In certain embodiments, cDNAs that encode antigens specific to the cancer to be treated or prevented are enriched in the pool of cDNAs or the cDNA library that is used with the methods of the invention. Any method known to the skilled artisan can be used to enrich for cDNAs that are specific to the cancer to be treated. In a specific embodiment, PCR-Select™ cDNA Subtraction Kit from BD Biosciences is used to enrich for cDNAs that encode antigens that are specific to the tumor. In certain embodiments, the cDNAs from the tumor cell or precancerous cell are enriched for cDNAs that are present in the tumor cell or precancerous cell but not in a cell from which the tumor cell or precancerous cell is derived.

In certain embodiments, one or more cDNAs are introduced into the nondendritic cell, wherein the cDNA encodes a tumor-associated antigen or a tumor-associated epitope. In certain embodiments, the cDNA encodes an antigen or epitope whose expression is upregulated in the cancer compared to non-cancerous cells. A tumor-associated epitope can be, e.g., a region of a protein that has a structure different from the wild-type protein due to a mutation, wherein the mutation is known to be associated with the cancer. In certain embodiments, one or more expression vectors are introduced into the nondendritic cell, wherein a tumor-associated antigen or a tumor-associated epitope can be expressed from the expression vector. In certain embodiments, one or more expression vectors are introduced into the nondendritic cell, wherein an antigen or an epitope that is upregulated in the cancer compared to a non-cancerous cell can be expressed from the expression vector. The non-dendritic cells with the cDNA(s) and/or the expression vector(s) are subsequently fused to an antigen presenting cell. The fusion cell can then be used to stimulate an immune response against the tumor-associated antigen or tumor-associated epitope or the antigen or epitope that is upregulated in a cancer cell compared to a non-cancerous cell.

The cDNAs can be transfected or microinjected into the non-dendritic cells by any technique known to the skilled artisan. In certain embodiments, the cDNAs are transfected into the nondendritic cells using, e.g., calcium phosphate transfection, DEAE-Dextran transfection, electroporation or liposome mediated transfection (see Chapter 9 of Short Protocols in Molecular Biology, Ausubel et al. (editors), John Wiley & Sons, Inc., 1999).

In certain embodiments, cDNAs that are synthesized from mRNA of an infectious agent are introduced into non-dendritic cells that are then fused with antigen presenting cells. Such fusion cells can be used to promote an immune response against the infectious agent from which the cDNA was obtained. The mRNA for synthesis of the cDNA can be obtained from the infectious agent or from a cell that is infected with the infectious agent. In particular, if the infectious agent is not known, cDNA is derived from a cell of the subject inflicted with the infectious disease.

The methods and sources of antigen-presenting cells and non-dendritic cells described in sections 4.1.1 and 4.1.2 for the fusion of non-dendritic cells and cells transfected with genomic DNA from a tumor cell or a precancerous cell can also be used for the fusion of antigen-presenting cells and non-dendritic cells transfected with cDNA from pre-cancerous cells or tumor cells. Target cancers that can be treated or prevented using the fusion cells of the invention are described below in Sections 4.12.

Any method can be used to identify and isolate those non-dendritic cells in which the cDNA has been introduced. In certain embodiments, DNA that encodes a marker gene is introduced concurrently with the cDNA into the non-dendritic cells. Cells that are positive for the marker gene also harbor the cDNA. Any marker gene known to the skilled artisan can be used. Illustrative examples of marker genes include genes whose gene products confer resistancy to a particular antibiotic to the cells (e.g., neomycine resistancy), genes whose gene products enable a cell to grow on a medium that lacks a substance that is normally required by this cell for growth, or genes whose gene products encode a visual marker. A visual marker that can be used with the methods of the invention is, e.g., GFP. Cells in which the DNA encoding the visual marker and the cDNA have been introduced can be isolated using FACS.

In certain embodiments of the invention, mRNA derived from a cancer cell or a cell of a precancerous lesion is directly introduced into a nondendritic cells instead of cDNA before fusion of the nondendritic cell to an antigen-presenting cell. Such fusion cells can then be used for the treatment and prevention of cancer as described above for fusions of antigen-presenting cells and non-dendritic cells containing cDNA derived from a cancer cell or a cell of a precancerous lesion. In certain embodiments, cDNA that has been prepared from mRNA isolated from a cancer cell or a cell of a precancerous lesion, or an infectious agent can be used to transcribe mRNA, which is then introduced into the non-dendritic cell. In certain embodiments, mRNA encoding a tumor-associated antigen is introduced into the non-dendritic cell. Tumor-associated antigens are described in section 4.8.

In certain embodiments of the invention, mRNA derived from an infectious agent is introduced into a nondendritic cells before fusion of the non-dendritic cell to an antigen-presenting cell. Such fusion cells can then be used for the treatment and prevention of an infectious disease as described herein for fusion cells. In certain embodiments, cDNA that has been prepared from mRNA isolated from an infectious agent can be used to transcribe mRNA, which is then introduced into the non-dendritic cell. In certain embodiments, mRNA encoding an antigen specific to an infectious agent is introduced into the non-dendritic cell. Tumor-associated antigens are described in section 4.8.

In certain embodiments, the antigen-presenting cells are mature dendritic cells (see, e.g., sections 4.5 and 4.9).

### 4.5 ANTIGEN PRESENTING CELLS

Antigen presenting cells, such as dendritic cells (DCs) can be isolated or generated from blood or bone marrow, or secondary lymphoid organs of the subject, such as but not limited to spleen, lymph nodes, tonsils, Peyer's patch of the intestine or bone marrow, by any of the methods known in the art. In a preferred embodiment, the dendritic cells used in the methods of the invention are terminally differentiated dendritic cells. In one embodiment, dendritic cells are isolated from human blood monocytes. In certain embodiments, the dendritic cells are autologous to the subject to whom the fusion cells of the present invention are to be administered. In alternative embodiments, the dendritic cells are allogeneic to the subject to whom the fusion cells of the present invention are to be administered. In certain embodiments, at least one MHC class I allele is shared between the antigen presenting cell and the subject to be treated. In certain embodiments, the antigen presenting cell is a universal antigen presenting cell (see section 4.7).

Immune cells obtained from such sources typically comprise predominantly recirculating lymphocytes and macrophages at various stages of differentiation and maturation. Dendritic cell preparations can be enriched by standard techniques (see e.g., Current Protocols in Immunology, 7.32.1-7.32.16, John Wiley and Sons, Inc., 1997). In one embodiment, for example, dendritic cells may be enriched by depletion of T cells and adherent cells, followed by density gradient centrifugation. Dendritic cells may optionally be further purified by sorting of fluorescently-labeled cells, or by using anti-CD83 mAb magnetic beads.

Alternatively, a high yield of a relatively homogenous population of dendritic cells can be obtained by treating dendritic cell progenitors present in blood samples or bone marrow with cytokines, such as granulocyte-macrophage colony stimulating factor (GM-CSF) and interleukin 4 (IL-4). Under such conditions, monocytes differentiate into dendritic cells without cell proliferation. Further treatment with an agent such as, but not limited to, TNFα stimulates terminal differentiation of dendritic cells.

In certain embodiments, the yield of dendritic cells can be increased by administering an effective amount of FLT3 ligand and to the individual from whom the dendritic cells are to be isolated (see, e.g., Fong et al., 2000, Altered Peptide Lig and Vaccination with Flt 3 Lig and Expanded Dendritic Cells from Tumor, Immunotherapy, Proc. Natl. Sci. USA 98(15):8809-14).

By way of example but not limitation, dendritic cells are obtained from blood monocytes according to standard methods (see, e.g., Sallusto et al., 1994, J. Exp. Med. 179:1109-1118). Leukocytes from healthy blood donors are collected by leukapheresis pack or buffy coat preparation using Ficoll-Paque density gradient centrifugation and plastic adherence. If mature dendritic cells are desired, the following protocol may be used to culture Dendritic cells. Cells are allowed to adhere to plastic dishes for 4 hours at 37 EC. Nonadherent cells are removed and adherent monocytes are cultured for 7 days in culture media containing 0.1 μg/ml granulocyte-macrophage colony stimulating factor and 0.05 μg/ml interleukin-4. In order to prepare dendritic cells, tumor necrosis factor-α is added on day 5 and cells are collected on day 7.

Dendritic cells obtained in this way characteristically express the cell surface marker CD83. In addition, such cells characteristically express high levels of MHC class II molecules, as well as cell surface markers CD1α, CD40, CD86, CD54, and CD80, but lose expression of CD14. Other cell surface markers characteristically include the T cell markers CD2 and CD5, the B cell marker CD7 and the myeloid cell markers CD13, CD32 (FcγR II), CD33, CD36, and CD63, as well as a large number of leukocyte-associated antigens

Optionally, standard techniques such as morphological observation and immunochemical staining, can be used to verify the presence of dendritic cells. For example, the purity of dendritic cells can be assessed by flow cytometry using fluorochrome-labeled antibodies directed against one or more of the characteristic cell surface markers noted above, e.g., CD83, HLA-ABC, HLA-DR, CD1α, CD40, and/or CD54. This technique can also be used to distinguish between and immature dendritic cells, using fluorochrome-labeled antibodies directed against CD14, which is present in immature, but not in mature, differentiated dendritic cells.

In certain embodiments, a universal antigen presenting cell as described in section 4.7 is used as an antigen presenting cell with the methods of the invention. In certain embodiments, a universal antigen presenting cell as described in section 4.7 is used as an antigen presenting cell to generate a fusion cell of the invention.

### 4.6 GENERATION OF FUSION CELLS

Non-dendritic cells can be fused to antigen presenting cells as follows. Cells are sterile-washed and fused according to any cell fusion technique in the art, provided that the fusion technique results in a mixture of fused cells suitable for injection into a mammal for prevention of cancer. In certain embodiments, electrofusion is used. Electrofusion techniques are well known in the art (Stuhler and Walden, 1994, Cancer Immunol. Immunother. 39:342-345; see Chang et al. (eds.), Guide to Electroporation and Electrofusion. Academic Press, San Diego, 1992).

In an illustrative embodiment, 5×10⁷ non-dendritic cells are used as starting material for the formation of fusion cells. In one embodiment, approximately 1×10⁶ to 1×10⁹ non-dendritic cells are used for formation of fusion cells. In another embodiment, 5×10⁷ to 2×10⁸ non-dendritic cells are used. In yet another embodiment, 1×10⁷ to 1×10¹⁰ non-dendritic cells are used. The use of other quantities of non-dendritic cells for preparation of fusion cells are within the scope of the invention.

In a specific illustrative non-limiting embodiment, the following protocol is used. In the first step, approximately 5×10⁷ non-dendritic cells into which the genomic DNA or cDNA has been introduced and 5×10⁷ dendritic cells are suspended in 0.3 M glucose and transferred into an electrofusion cuvette. The sample is dielectrophoretically aligned to form cell-cell conjugates by pulsing the cell sample at 100 V/cm for 5-10 sec. Optionally, alignment may be optimized by applying a drop of dielectrical wax onto one aspect of the electroporation cuvette to “inhomogenize” the electric field, thus directing the cells to the area of the highest field strength. In a second step, a fusion pulse is applied. Various parameters may be used for the electrofusion. For example, in one embodiment, the fusion pulse may be from a single to a triple pulse. In another embodiment, electrofusion is accomplished using from 500 to 1500 V/cm, preferably, 1,200 V/cm at about 25 μF.

In a preferred embodiment, the non-dendritic cells are autologous to the patient to whom the fusion cells of the present invention are to be administered. In another preferred embodiment, the antigen presenting cells are autologous to the patient to whom the fusion cells of the present invention are to be administered. In an even more preferred embodiment, both the non-dendritic cells and the antigen presenting cells are autologous to the patient to whom the fusion cells of the present invention are administered.

In another embodiment, the following protocol is used. First, bone marrow is isolated and red cells lysed with ammonium chloride (Sigma, St. Louis, Mo.). Lymphocytes, granulocytes and antigen presenting cells are depleted from the bone marrow cells and the remaining cells are plated in 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 pg/ml streptomycin, 10 ng/ml recombinant granulocyte-macrophage colony stimulating factor (GM-CSF; Becton Dickinson, San Jose, Calif.) and 30 U/ml recombinant interleukin-4 (IL-4; Becton Dickinson). Second, on day 5 of culture, nonadherent and loosely adherent cells are collected and replated on 100-mm petri dishes (1×10⁶ cells/mi; 10 ml/dish). Next, GM-CSF and IL-4 in RPMI medium are added to the cells and 1×10⁶ DCs are mixed with 3×10⁶ irradiated (50 Gy, Hitachi MBR-1520R, dose rate: 1.1 Gy/min) pre-cancerous non-dendritic cells. After 48 hr, fusion is initiated by adding dropwise over 60 sec, 500 μl of a 50% solution of polyethylene glycol (PEG 1500; Sigma, St. Louis, Mo.). The fusion is stopped by stepwise addition of 30 ml. of serum-free RPMI medium. Fusion cells are plated in 100-mm petri dishes in the presence of GM-CSF and IL-4 in RPMI medium for 48 hours.

In another embodiment, the dendritic cell and non-dendritic cell are fused as described above. Subsequently, the fused cells are transformed or transfected with genetic material which encodes a molecule which stimulates a CTL and/or humoral immune response. In a preferred embodiment, the genetic material is mRNA encoding IL-12. Preferred methods of transfection include electroporation or transformation or transfection in the presence of cationic polymers.

The extent of fusion cell formation within a population of non-dendritic cells and antigen presenting cells can be determined by a number of diagnostic techniques known in the art. In one embodiment, for example, hybrids are characterized by labeling antigen presenting cells and non-dendritic cells with red and green intracellular fluorescent dyes, respectively, and detection the emission of both colors. Samples of antigen presenting cells without non-dendritic cells, and non-dendritic cells without antigen presenting cells can be used as negative controls, as well as a mixture of non-fused pre-cancerous non-dendritic cells and antigen presenting cells.

In certain embodiments, before administration of fusions cells (with or without the co-administration of an immunostimulatory molecule) to a mammal, the fusion cells are inactivated, for example, by irradiation, to prevent proliferation of the fusion cells. Preferably, the fusion cell population is irradiated at 200 Gy, and injected without further selection. In one embodiment, the fusion cells prepared by this method comprise approximately 10 and 20% of the total cell population. In yet another embodiment, the fusion cells prepared by this method comprise approximately 5 to 50% of the total cell population.

### 4.7 MOLECULAR VACCINES FROM UNIVERSAL ANTIGEN PRESENTING CELLS

In certain embodiments, genomic DNA from a precancerous cell or cancer cell is introduced into a universal antigen presenting cell. Universal antigen presenting cells are prepared by recombinantly expressing one or more co-stimulatory molecules (e.g., B7, ICAM-I and/or ICAM-II) in a cell. In certain embodiments, the universal antigen presenting cell is prepared by recombinantly expressing one or more of the following molecules in a cell: B7, ICAM-I, ICAM-II, MHC class I, MHC class II and/or LFA-3. Any method known to the skilled artisan can be used to determine which of the co-stimulatory molecules is expressed endogenously by the cell. The cell is then engineered to express certain additional co-stimulatory molecules accordingly.

In certain embodiments, the universal antigen presenting cell is prepared to recombinantly express ICAM-I. In certain embodiments, the universal antigen presenting cell is prepared to recombinantly express ICAM-I and MHC class I. In certain embodiments, the universal antigen presenting cell is prepared to recombinantly express ICAM-I, MHC class I, and B7. In certain embodiments, the universal antigen presenting cell is prepared to recombinantly express ICAM-I, MHC class II and MHC class I. In certain embodiments, the universal antigen presenting cell is prepared to recombinantly express ICAM-I, MHC class I, MHC class II and B7. In certain embodiments, the universal antigen presenting cell is prepared to recombinantly express ICAM-I and ICAM-IL. In certain embodiments, the universal antigen presenting cell is prepared to recombinantly express ICAM-I, ICAM-IL and MHC class I. In certain embodiments, the universal antigen presenting cell is prepared to recombinantly express ICAM-I, ICAM-II, MHC class I, and B7. In certain embodiments, the universal antigen presenting cell is prepared to recombinantly express ICAM-I, ICAM-II, MHC class II and MHC class I. In certain embodiments, the universal antigen presenting cell is prepared to recombinantly express ICAM-I, ICAM-II, MHC class I, MHC class II and B7. In certain embodiments, the cells are engineered to recombinantly express LFA-3. In certain embodiments, the universal antigen presenting cell is engineered to recombinantly express B7.1 (CD80) and/or B7.2 (CD86). In certain embodiments, the molecules that are recombinantly expressed in a cell to generate a universal antigen presenting cell are encoded by an allele of that gene that is identical to an allele of that gene from the subject that is to be treated. In certain embodiments, the antigen presenting cell is matched for major histocompatibility complex (MHC) with the subject to be treated. In certain, more specific embodiments, at least one MHC class I allele is common between the recipient of the fusion cells and the universal antigen-presenting cell.

Costimulatory molecules are involved in the interaction between receptor-ligand pairs expressed on the surface of antigen presenting cells and T cells, respectively. One exemplary receptor-ligand pair is the B7 co-stimulatory molecules on the surface of dendritic cells and its counter-receptor CD28 or CTLA-4 on T cells (Freeman et al. (1993) Science 262: 909-911; Young et al. (1992) J. Clin. Invest 90: 229; and Nabavi et al. Nature 360: 266). Other important costimulatory molecules are CD40, CD54, CD80, and CD86, which can also be used with the methods and compositions of the invention, alone or in combination.

A universal antigen presenting cell can be prepared from any cell type. In certain embodiments, the cell to be used to generate a universal antigen presenting cell is derived from the same species as the species of the subject that is to be treated. In certain embodiments, the universal antigen presenting cell is prepared from cell that can readily be transfected. In certain embodiments of the invention, the universal antigen presenting cell is prepared from a cell that grows readily in culture. In a specific, illustrative embodiment, a universal antigen presenting cell of the invention is a 293P cell. In certain embodiments, the universal antigen presenting cell is engineered to recombinantly express a cytokine, such as, but not limited to, IL-12.

Any method known to the skilled artisan can be used to express the co-stimulatory molecules in a cell to generate a universal antigen presenting cell. In certain embodiments, cells are transiently transfected with expression vectors encoding the costimulatory molecules. In other embodiments, cells are permanently transfected to express the costimulatory molecules. The DNA of the expression constructs can be introduced into the cells by any method known to the skilled artisan. In exemplary embodiments, the DNA is introduced by calcium phosphate transfection, DEAE-Dextran transfection, electroporation or liposome mediated transfection (see Chapter 9 of Short Protocols in Molecular Biology, Ausubel et al. (editors), John Wiley & Sons, Inc., 1999). In other embodiments, the DNA molecules encoding the costimulatory factors are introduced into the cells by microinjection. Any promoter suitable for expression in the particular cell type that is used to generate the universal antigen presenting cells can be used for the expression constructs of the costimulatory factors. Vectors for expression of the costimulatory factor(s) can be plasmid, viral, or others known in the art, used for replication and expression in mammalian cells. Expression of the costimulatory factor(s) can be by any promoter known in the art to act in the cells to be used to generate the universal antigen presenting cells. Such promoters can be inducible or constitutive. Such promoters include but are not limited to: the SV40 early promoter region (Bemoist and Chambon, 1981, Nature 290, 304-310), the promoter contained in the 3 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be used to transfect the cell.

The universal antigen presenting cells that comprise genomic DNA of a cancer cell or a precancerous cell can be used as vaccines against cancer as described herein.

In certain embodiments, universal antigen presenting cells can be transfected or microinjected with complementary DNA molecules (“cDNAs”) that have been synthesized from mRNA that has been extracted from a tumor cell or a pre-cancerous cell (see section 4.4).

In certain embodiments, universal antigen presenting cells can be transfected or microinjected with complementary DNA molecules (“cDNAs”) or a cDNA library that have been synthesized from mRNA that has been extracted from an infectious agent or with the genomic DNA of an infection agent.

Any technique known to the skilled artisan can be used to obtain an allele of a MHC class I, MHC class II or co-stimulatory molecule from the subject to be treated. In a specific embodiment, DNA is obtained from the subject to be treated and the genes encoding MHC class I, MHC class II and/or co-stimulatory molecule are amplified using PCR. In another embodiment, RNA is isolated from the subject to be treated and the open reading frames encoding MHC class I, MHC class II and/or co-stimulatory molecules are obtained using RT-PCR. The DNA can then be cloned into a vector with a suitable promoter and subsequently transfected into a cell to generate a universal antigen presenting cell. As the skilled artisan will appreciate, the suitability of the promoter depends on the cell-type that is used to generate the universal antigen presenting cell.

The present invention also relates to methods for generating universal antigen presenting cells, fusion cells with antigen presenting cells and non-dendritic cells, and generating universal antigen-presenting cell that contain genomic DNA of a tumor cell or cDNA derived from mRNA isolated from a tumor cell. The invention also relates to the cells generated by these methods.

In certain embodiments of the invention, mRNA derived from a cancer cell, a cell of a precancerous lesion, or an infectious agent is introduced into a universal antigen-presenting cell. Such universal antigen-presenting cells can then be used for the treatment and prevention of cancer or an infectious disease, respectively, as described above for fusion cells. In certain embodiments, cDNA that has been prepared from mRNA isolated from a cancer cell, a cell of a precancerous lesion or an infectious agent can be used to transcribe mRNA, which is then introduced into a universal antigen-presenting cell. In certain embodiments, mRNA encoding a tumor-associated antigen or an antigen specific to an infectious agent is introduced into a universal antigen-presenting cell.

### 4.8 FUSION OF ANTIGEN-PRESENTING CELLS WITH NON-DENDRITIC CELLS THAT CONTAIN DNA ENCODING A TUMOR-ASSOCIATED ANTIGEN

In certain embodiments, the invention provides a method for treating or preventing cancer in a subject comprising administering to the subject fusion cells, wherein such fusion cells are generated by fusing antigen presenting cells with non-dendritic cells, wherein the non-dendritic cell contain one or more expression constructs encoding one or more tumor-associated antigens.

Tumor-associated antigens (or cancer-associated antigens) include, but are not limited to, p53 and mutants thereof, Ras and mutants thereof, a Bcr/Abl breakpoint peptide, HER-2/neu, HPV E6, HPV E7, carcinoembryonic antigen, MUC-1, MAGE-1, MAGE-3, BAGE, GAGE-1, GAGE-2, N-acetylglucosaminyltransferase-V, p15, gp100, MART-1/MelanA, tyrosinase, TRP-1, beta.-catenin, MUM-1 and CDK-4. Other tumor-associated tumor-antigens include KS ¼ pan-carcinoma antigen (Perez and Walker, 1990, J. Immunol. 142:3662-3667; Bumal, 1988, Hybridoma 7(4):407-415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63) and prostate specific membrane antigen.

Any method known to the skilled artisan can be used to express the tumor-associated antigen in a non-dendritic cell. In certain embodiments, non-dendritic cells are transiently transfected with expression vectors encoding the tumor-associated antigen. In other embodiments, non-dendritic cells are permanently transfected to express the tumor-associated antigen. The DNA of the expression constructs can be introduced into the cells by any method known to the skilled artisan. In exemplary embodiments, the DNA is introduced by calcium phosphate transfection, DEAE-Dextran transfection, electroporation or liposome mediated transfection (see Chapter 9 of Short Protocols in Molecular Biology, Ausubel et al. (editors), John Wiley & Sons, Inc., 1999). In other embodiments, the DNA molecules encoding the tumor-associated antigens are introduced into the cells by microinjection. Any promoter suitable for expression in the particular cell type of non-dendritic cells that is used to generate the fusion cells can be used for the expression constructs of the tumor-associated antigens. Such promoters can be inducible or constitutive. Such promoters include but are not limited to: the SV40 early promoter region (Bemoist and Chambon, 1981, Nature 290, 304-310), the promoter contained in the 3 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Vectors for expression of the tumor-associated antigens can be plasmid, viral, or others known in the art, used for replication and expression in mammalian cells. Any type of plasmid, cosmid, YAC or viral vector can be used to prepare the recombinant DNA construct which can be used to transfect the cell.

In a specific embodiment, an expression vector encoding a tumor-associated antigen is introduced into a non-dendritic cell of the intended recipient, the non-dendritic cell is subsequently fused to a dendritic cell and the resulting fusion cell is administered to the recipient.

In certain embodiments, a cDNA or an mRNA encoding an antigen associated with or specific to an infectious agent is introduced into the non-dendritic cell.

In certain embodiments, the invention provides compositions comprising fusion cells, wherein such fusion cells are generated by fusing antigen presenting cells, such as dendritic cells or universal antigen presenting cells with non-dendritic cells, wherein the non-dendritic cell contain one or more expression constructs encoding one or more tumor-associated antigens. In certain embodiments, the invention provides compositions comprising universal antigen presenting cells 4.7 containing one or more expression constructs encoding one or more tumor-associated antigens.

In certain embodiments, the invention provides compositions comprising fusion cells, wherein such fusion cells are generated by fusing antigen presenting cells, such as dendritic cells or universal antigen presenting cells with non-dendritic cells, wherein the non-dendritic cell contain one or more expression constructs encoding one or more antigens specific to an infectious agent. In certain embodiments, the invention provides compositions comprising universal antigen presenting cells 4.7 containing one or more expression constructs encoding one or more antigens specific to an infectious agent.

### 4.9 EDUCATION OF IMMUNE EFFECTOR CELLS WITH FUSION CELLS OR UNIVERSAL ANTIGEN PRESENTING CELLS

In certain embodiments of the invention, a fusion cell of the invention or a universal antigen presenting cell of the invention can be used to generate antigen-specific immune effector cells. Immune effector cells include, but are not limited to, B cells, monocytes, macrophages, NK cells and T cells. In certain embodiments, a fusion cell of the invention or a universal antigen presenting cell of the invention can be used to educate an immune effector cell. In certain embodiments, a fusion cell of the invention or a universal antigen presenting cell of the invention can be used to generate an antigen-specific immune effector cell from an immune effector cell that is not antigen-specific. In certain embodiments, a method of the invention relates to the expansion of immune effector cells at the at the expense of fusion cells of the invention in culture. In certain embodiments, the method comprises coculturing an immune effector cell with a fusion cell of the invention.

Fusion cells of the invention that can be used to educate immune effector cells and/or to expand or generate antigen-specific immune effector cells can be formed by fusing an antigen-presenting cell, such as a dendritic cells or universal antigen presenting cells, and a non-dendritic cell, wherein the non-dendritic cell comprises genomic DNA extracted from a cancer cell or a cell of a precancerous lesion; cDNA or a cDNA library derived from a cancer cell or a cell of a precancerous lesion; one or more expression constructs encoding a tumor-associated antigen; genomic DNA extracted from an infectious agent; genomic DNA extracted from a cell infected with an infectious agent; cDNA derived from an infectious agent; cDNA derived from a cell infected with an infectious agent; one or more expression constructs encoding an antigen specific to an infectious agent; mRNA derived from a cancer cell, cell of a precancerous lesion, or infectious agent; or mRNA transcribed from cDNA derived from a cancer cell, cell of a precancerous lesion, or infectious agent. In certain embodiments, the fusion cells of the invention express one or more antigens of the cancer to be treated or prevented. In certain embodiments, the fusion cells of the invention express one or more antigens of the infectious agent to be treated or prevented. In certain embodiments, the antigen-presenting cell that is used to prepare the fusion cell is a universal antigen-presenting cell.

In certain embodiments, a universal antigen presenting cell (see section 4.7) that comprises genomic DNA extracted from a cancer cell or a cell of a precancerous lesion; cDNA or a cDNA library derived from a cancer cell or a cell of a precancerous lesion; one or more expression constructs encoding a tumor-associated antigen; genomic DNA extracted from an infectious agent; genomic DNA extracted from a cell infected with an infectious agent; cDNA derived from an infectious agent; cDNA derived from a cell infected with an infectious agent; one or more expression constructs encoding an antigen specific to an infectious agent; mRNA derived from a cancer cell, cell of a precancerous lesion, or infectious agent; or mRNA transcribed from cDNA derived from a cancer cell, cell of a precancerous lesion, or infectious agent can be used to educate an immune effector cell and/or to expand or generate antigen-specific immune effector cells.

In certain embodiments, a fusion cell or universal antigen presenting cell is used to educate immune effector cells and/or to expand or generate antigen-specific immune effector cells by contacting the fusion cell or universal antigen presenting cell with an immune effector cell.

In certain embodiments, the fusion cell or the universal antigen presenting cell that is used to educate immune effector cells and/or to expand or generate antigen-specific immune effector cells expresses one ore more antigens that are associated with a cancer or a precancerous lesion. In a more specific embodiment, the antigen is specific to the cancer or the precancerous lesion. In certain, more specific embodiments, the antigen is expressed on the cell-surface of the fusion cell or the universal antigen presenting cell.

In certain embodiments, the fusion cell or the universal antigen presenting cell that is used to educate immune effector cells and/or to expand or generate antigen-specific immune effector cells expresses one ore more antigens that are associated with an infectious agent. In a more specific embodiment, the antigen is specific to an infectious agent. In certain, more specific embodiments, the antigen is expressed on the cell-surface of the fusion cell or the universal antigen presenting cell.

In certain embodiments, the immune effector cell and the fusion cell of the invention share at least one MHC class I allele in common. In certain embodiments, the immune effector cell and the fusion cell of the invention are allogeneic.

In certain embodiments, the fusion cell of the invention or the universal antigen presenting cell is mixed with naive immune effector cells. In more specific embodiments, the immune effector cells specifically recognize tumor cells and have been enriched from a tumor biopsy sample of the patient to be treated. Optionally, the cells may be cultured in the presence of a cytokine, for example IL-2 or IL-12. In certain embodiments, the culture conditions are such that the antigen-specific immune effector cells proliferate at a higher rate than the fusion cells or universal antigen-presenting cells. In certain embodiments, fusion cells or universal antigen-presenting cells are added to the co-culture one or more times.

In certain embodiments, the invention also relates to the immune effector cells that have been obtained by expanding immune effector cells at the expense of fusion cells of the invention.

Exemplary methods for the expansion of immune effector cells are described in U.S. Application Publication No. 2002/0,041,868, published Apr. 11, 2002 (Application No. 09/782,492 filed Feb. 12, 2001), which is incorporated by reference herein in its entirety.

In certain embodiments, the invention relates to expanding immune effector cells at the expense of fusion cells, wherein the fusion cells are fusions between a mature dendritic cell and a tumor cell. The fusion cells can be generated by any method known to the skilled artisan, e.g., as described in section 4.6.

By way of example but not limitation, mature dendritic cells can be obtained from blood monocytes as follows: peripheral blood monocytes are obtained by standard methods (see, e.g., Sallusto et al., 1994, J. Exp. Med. 179:1109-1118). Leukocytes from healthy blood donors are collected by leukapheresis pack or buffy coat preparation using Ficoll-Paque density gradient centrifugation and plastic adherence. Cells are allowed to adhere to plastic dishes for 4 hours at 37° C. Nonadhering cells are removed and adherent monocytes are cultured for 7 days in culture media containing 0.1 μg/ml granulocyte-monocyte colony stimulating factor and 0.05 μg/ml interleukin-4. In order to prepare mature dendritic cells, tumor necrosis factor-α (TNF-α) is added, preferably on day 5, and cells are collected on day 7.

Dendritic cells obtained in this way characteristically express the cell surface marker CD83. In addition, such cells characteristically express high levels of MHC class II molecules, as well as cell surface markers CD1α, CD40, CD86, CD54, and CD80, but lose expression of CD14. Other cell surface markers characteristically include the T cell markers CD2 and CD5, the B cell marker CD7 and the myeloid cell markers CD13, CD32 (FcγR II), CD33, CD36, and CD63, as well as a large number of leukocyte-associated antigens.

Optionally, standard techniques such as morphological observation and immunochemical staining, can be used to verify the presence of dendritic cells. For example, the purity of dendritic cells can be assessed by flow cytometry using fluorochrome-labeled antibodies directed against one or more of the characteristic cell surface markers noted above, e.g., CD83, HLA-ABC, HLA-DR, CD1α, CD40, and/or CD54. This technique can also be used to distinguish between mature and immature DCs, using fluorochrome-labeled antibodies directed against CD14, which is present in immature, but not mature DCs.

The invention also relates to methods of treatment using the immune effector cells that have been generated using a fusion cell or a universal antigen presenting cell of the invention. In certain embodiments, a method comprises administering an immune effector cell to a subject wherein the immune effector cell has been expanded and/or generated or educted using a fusion cell or a universal antigen presenting cell.

### 4.10 IMMUNE CELL ACTIVATING MOLECULES

The present invention provides a composition which comprises first, a fusion cell derived from the fusion of a dendritic and a non-dendritic cell, wherein genomic DNA of a tumor cell or a pre-cancerous cell has been introduced into the non-dendritic cell before fusion, and in certain embodiments, further comprise a cytokine or other molecule which can stimulate or induce a cytotoxic T cell (CTL) response and/or a humoral response.

In a preferred embodiment, the CTL stimulating molecule is IL-12. IL-12 plays a major role in regulating the migration and proper selection of effector cells in an immune response. The IL-12 gene product generally polarizes the immune response toward the TH₁ subset of T helper cells and strongly stimulates CTL activity. As elevated doses of IL-12 exhibits toxicity when administered systemically, IL-12 is preferably administered locally. Additional modes of administration are described below in Section 4.13.

Expression of IL-12 receptor β2 (IL-12R-β2) is necessary for maintaining IL-12 responsiveness and controlling TH₁ lineage commitment. Furthermore, IL-12 signaling results in STAT4 activation, i.e., measured by an increase of phosphorylation of STAT4, and interferon-γ (IFN-γ) production. Thus, in one embodiment, the present invention contemplates the use of a molecule, which is not IL-12, which can activate STAT4, for example a small molecule activator of STAT4 identified by the use of combinatorial chemistry.

In certain embodiments, a molecule that increases the production of interferon-γ other than IL-12 is used in combination with the fusion cells.

In an alternative embodiment, the immune stimulating molecule is IL-18. In yet another embodiment, the immune stimulating molecule is IL-15. In yet another embodiment, the immune stimulating molecule is interferon-γ.

In another embodiment, the patient to be treated is administered any combination of molecules or cytokines described herein which stimulate or induce a CTL and/or a humoral immune response.

In a less preferred embodiment, to increase the cytotoxic T-cell pool, i.e., the TH₁ cell subpopulation, anti-IL-4 antibodies can be added to inhibit the polarization of T-helper cells into TH₂ cells, thereby creating selective pressure toward the TH₁ subset of T-helper cells. Further, anti-IL-4 antibodies can be administered concurrent with the administration of IL-12, to induce the TH cells to differentiate into TH₁ cells. After differentiation, cells can be washed, resuspended in, for example, buffered saline, and reintroduced into a patient via, preferably, intravenous administration.

In another embodiment, to enhance a humoral response, IL-4 is added to stimulate production of TH₂ helper T-cells and promote synthesis of antibodies that specifically bind to the pre-cancerous cells or tumor cells of the treated individual.

The present invention also pertains to variants of the above-described interleukins. Such variants have an altered amino acid sequence which can function as agonists (mimetics) to promote a CTL and/or humoral immune response. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a molecule capable of stimulating a CTL and/or humoral immune response can be identified by screening combinatorial libraries of mutants, e.g., truncation mutants, for agonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of IL-12 from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem., 53:323; Itakura et al., 1984, Science, 198:1056; Ike et al., 1983, Nucleic Acid Res., 11:477).

In addition, libraries of fragments of the coding sequence of an interleukin capable of promoting a CTL and/or humoral immune response can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of an interleukin capable of promoting a CTL and/or humoral immune response (Arkin and Yourvan, 1992, Proc. Natl. Acad. Sci. USA, 89:7811-7815; Delgrave et al., 1993, Protein Engineering, 6(3):327-331).

### 4.11 ASSAYS FOR MEASURING AN IMMUNE RESPONSE

The fusion cell or universal antigen presenting cell of the invention can be assayed for immunogenicity using any method known in the art. By way of example but not limitation, one of the following procedures can be used.

A humoral immune response can be measured using standard detection assays including but not limited to an ELISA, to determine the relative amount of antibodies which recognize the target antigen in the sera of a treated subject, relative to the amount of antibodies in untreated subjects. A CTL response can be measured using standard immunoassays including chromium release assays as described herein. More particularly, a CTL response is determined by the measurable difference in CTL activity upon administration of a stimulator, relative to CTL activity in the absence of a stimulator.

### 4.11.1 MLTC ASSAY

The fusion cell and universal antigen presenting cell of the invention may be tested for immunogenicity using a mixed lymphocyte T cell culture (MLTC) assay. For example, 1×10⁷ fusion cells are y-irradiated, and mixed with T lymphocytes. At various intervals the T lymphocytes are tested for cytotoxicity in a 4 hour ⁵¹Cr-release assay (see Palladino et al., 1987, Cancer Res. 47:5074-5079). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 500 μCi ⁵¹Cr/ml for one hour at 37EC. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelletted by centrifugation at 200×g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

### 4.11.2 ANTIBODY RESPONSE ASSAY

In one embodiment of the invention, the immunogenicity of fusion cells or universal antigen presenting cells is determined by measuring antibodies produced in response to the vaccination, by an antibody response assay, such as an enzyme-linked immunosorbent assay (ELISA) assay. Methods for such assays are well known in the art (see, e.g., Section 2.1 of Current Protocols in Immunology, Coligan et al. (eds.), John Wiley and Sons, Inc. 1997). In one mode of the embodiment, microtitre plates (96-well Immuno Plate II, Nunc) are coated with 50 μl/well of a 0.75 μg/ml solution of a purified pre-cancerous cell used in the composition in PBS at 4 EC for 16 hours and at 20 EC for 1 hour. The wells are emptied and blocked with 200 μl PBS-T-BSA (PBS containing 0.05% (v/v) TWEEN 20 and 1% (w/v) bovine serum albumin) per well at 20 EC for 1 hour, then washed 3 times with PBS-T. Fifty μl/well of plasma or CSF from a vaccinated animal (such as a model mouse or a human patient) is applied at 20 EC for 1 hour, and the plates are washed 3 times with PBS-T. The antigen antibody activity is then measured calorimetrically after incubating at 20 EC for 1 hour with 50 μ/well of sheep anti-mouse or anti-human immunoglobulin, as appropriate, conjugated with horseradish peroxidase diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a photometer at 492 nm (ref. 620 nm), using standard techniques.

### 4.11.3 CYTOKINE DETECTION ASSAYS

The CD4⁺ T cell proliferative response to the fusion cell or universal antigen presenting cell may be measured by detection and quantitation of the levels of specific cytokines. In one embodiment, for example, intracellular cytokines may be measured using an IFN-γ detection assay to test for immunogenicity of the fusion cell-cytokine composition. In an example of this method, peripheral blood mononuclear cells from a patient treated with the fusion cell-cytokine composition are stimulated with peptide antigens such as mucin peptide antigens or Her2/neu derived epitopes. Cells are then stained with T cell-specific labeled antibodies detectable by flow cytometry, for example FITC-conjugated anti-CD8 and PerCP-labeled anti-CD4 antibodies. After washing, cells are fixed, permeabilized, and reacted with dye-labeled antibodies reactive with human IFN-γ (PE- anti-IFN-γ). Samples are analyzed by flow cytometry using standard techniques.

Alternatively, a filter immunoassay, the enzyme-linked immunospot assay (ELISPOT) assay, may be used to detect specific cytokines surrounding a T cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of mononuclear blood cells, containing cytokine-secreting cells, obtained from a patient vaccinated with fusion cells or fusion cells and an immune stimulator such as a cytokine composition, is diluted into the wells of the microtitre plate. A labeled, e.g., biotin-labeled, secondary anti-cytokine antibody is added. The antibody-cytokine complex can then be detected, e.g. by enzyme-conjugated streptavidin, and cytokine-secreting cells will appear as “spots” by visual, microscopic, or electronic detection methods.

### 4.11.4 TETRAMER STAINING ASSAY

In another embodiment, the “tetramer staining” assay (Altman et al., 1996, Science 274: 94-96) may be used to identify antigen-specific T-cells. In one embodiment, an MHC molecule containing a specific peptide antigen, such as a tumor-associated antigen, is multimerized to make soluble peptide tetramers and labeled, for example, by complexing to streptavidin. The MHC complex is then mixed with a population of T cells obtained from a patient treated with a fusion cell composition. Biotin is then used to stain T cells which express the antigen of interest, ie., the tumor-associated antigen.

Cytotoxic T-cells are immune cells which are CD8 positive and have been activated by antigen presenting cells (APCs), that have processed and are displaying an antigen of a target cell. The antigen presentation, in conjunction with activation of co-stimulatory molecules such as B-7/CTLA-4 and CD40, leads to priming of the T-cell against the target, resulting in destruction of cells expressing the antigen.

Cytotoxic T-cells, generally characterized as expressing CD8, also secreted TNF-β, perforin, and IL-2. A cytotoxic T cell response can be measured in various assays, including but not limited to increased target cell lysis in ⁵¹Cr release assays using T-cells from treated subjects, in comparison to T-cells from untreated subjects, as shown in the examples herein, as well as measuring an increase in the levels of IFN-γ and IL-2 in treated subjects relative to untreated subjects.

### 4.12 TARGET CANCERS

The cancers and oncogenic diseases that can be prevented, as well as the pre-cancerous lesions, which lead to the development of those cancers and oncogenic diseases, that can be prevented and treated, using the fusion cells of the present invention include, but are not limited to: human sarcomas and carcinomas, e.g., renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

### 4.13 PHARMACEUTICAL PREPARATIONS AND METHODS OF ADMINISTRATION

The composition formulations of the invention comprise an effective immunizing amount of the fusion cells or universal antigen presenting cells which are to be administered either without or with one or more molecules, such as but not limited to cytokines, that are capable of stimulating a CTL and/or humoral immune response. The fusion cells of the pharmaceutical compositions of the invention can be fusion cells formed by fusing an antigen-presenting cell, such as a dendritic cells or universal antigen presenting cells, and a non-dendritic cell, wherein the non-dendritic cell comprises genomic DNA extracted from a cancer cell or a cell of a precancerous lesion; cDNA or a cDNA library derived from a cancer cell or a cell of a precancerous lesion; one or more expression constructs encoding a tumor-associated antigen; genomic DNA extracted from an infectious agent; genomic DNA extracted from a cell infected with an infectious agent; cDNA derived from an infectious agent; cDNA derived from a cell infected with an infectious agent; one or more expression constructs encoding an antigen specific to an infectious agent; mRNA derived from a cancer cell, cell of a precancerous lesion, or infectious agent; or mRNA transcribed from cDNA derived from a cancer cell, cell of a precancerous lesion, or infectious agent. In certain embodiments, the fusion cells of the invention express one or more antigens of the cancer to be treated or prevented. In certain embodiments, the fusion cells of the invention express one or more antigens of the infectious agent to be treated or prevented.

In certain embodiments, the invention provides a universal antigen presenting cell (see section 4.7). In certain embodiments of the invention, a universal antigen presenting cell of the invention comprises genomic DNA extracted from a cancer cell or a cell of a precancerous lesion; cDNA or a cDNA library derived from a cancer cell or a cell of a precancerous lesion; one or more expression constructs encoding a tumor-associated antigen; genomic DNA extracted from an infectious agent; genomic DNA extracted from a cell infected with an infectious agent; cDNA derived from an infectious agent; cDNA derived from a cell infected with an infectious agent; one or more expression constructs encoding an antigen specific to an infectious agent; mRNA derived from a cancer cell, cell of a precancerous lesion, or infectious agent; or mRNA transcribed from cDNA derived from a cancer cell, cell of a precancerous lesion, or infectious agent. The genomic DNA or cDNA or expression constructs can be introduced into the universal antigen presenting cell by any method known to the skilled artisan.

Suitable preparations of fusion cell or fusion cell-cytokine compositions include injectable formulations that are, preferably, liquid solutions.

Many methods may be used to introduce the composition formulations of the invention; these include but are not limited to subcutaneous injection, intralymphatically, intradermal, intramuscular, intravenous, and via scarification (scratching through the top layers of skin, e.g., using a bifurcated needle). Preferably, fusion cell and fusion cell-cytokine compositions are injected intradermally.

In addition, if desired, the composition preparation may also include minor amounts of auxiliary substances such as wetting or emulsifying agents, pH buffering agents, and/or compounds which enhance the effectiveness of the composition. The effectiveness of an auxiliary substances may be determined by measuring the induction of antibodies directed against a fusion cell.

The manual to which the composition is administered is preferably a human, but can also be a non-human animal including but not limited to cows, horses, sheep, pigs, fowl (e.g., chickens), goats, cats, dogs, hamsters, mice and rats.

### 4.14 EFFECTIVE DOSE

The compositions of the present invention can be administered to a patient at therapeutically effective doses to prevent or treat cancer or a precancerous lesion. A therapeutically effective amount refers to that amount of the fusion cells sufficient to prevent or ameliorate the symptoms of such a disease or disorder, such as, e.g., regression of a pre-cancerous lesion or prevention of formation of such lesions in a person, particularly an individual at risk of developing cancer. Effective doses (immunizing amounts) of the compositions of the invention may also be extrapolated from dose-response curves derived from animal model test systems. The precise dose of fusion cells to be employed in the composition formulation will also depend on the particular type of disorder being prevented. For example, if a tumor is to be prevented from developing, the aggressiveness of the tumor is an important consideration when considering dosage. Other important considerations are the route of administration, and the nature of the patient. Thus the precise dosage should be decided according to the judgment of the practitioner and each patient's circumstances, e.g., the immune status of the patient, according to standard clinical techniques.

In a preferred embodiment, for example, to prevent formation of a human tumor, a fusion cell or fusion cell-cytokine composition, comprising non-dendritic pre-cancerous cells of the patient fused to antigen presenting cells are administered at a site away from the pre-cancerous lesion, preferably near lymph tissue. The administration of the composition may be repeated after an appropriate interval, e.g., every 3-6 months, using approximately 1×10⁸ cells per administration.

The present invention thus provides a method of immunizing a mammal, and preventing or treating development of a pre-cancerous lesion development or progression thereof in a mammal, comprising administering to the mammal a therapeutically effective amount of a fusion cell or a fusion cell-cytokine composition of the present invention.

In certain embodiments, at least 10⁴ fusion cells are administered per kg body weight of the subject to be treated. In certain embodiments, at least 5×10⁴ fusion cells are administered per kg body weight of the subject to be treated. In certain embodiments, at least 10⁵ fusion cells are administered per kg body weight of the subject to be treated. In certain embodiments, at least 5×10⁵ fusion cells are administered per kg body weight of the subject to be treated. In certain embodiments, at least 10⁶ fusion cells are administered per kg body weight of the subject to be treated. In certain embodiments, at least 5×10⁶ fusion cells are administered per kg body weight of the subject to be treated. In certain embodiments, at least 10⁷ fusion cells are administered per kg body weight of the subject to be treated. In certain embodiments, at least 5×10⁷ fusion cells are administered per kg body weight of the subject to be treated. In certain embodiments, at least 10⁸ fusion cells are administered per kg body weight of the subject to be treated. In certain embodiments, at least 5×10⁸ fusion cells are administered per kg body weight of the subject to be treated. In certain embodiments, at least 10⁹ fusion cells are administered per kg body weight of the subject to be treated.

In certain embodiments, at most 10⁴ fusion cells are administered per kg body weight of the subject to be treated. In certain embodiments, at most 5×10⁴ fusion cells are administered per kg body weight of the subject to be treated. In certain embodiments, at most 10⁵ fusion cells are administered per kg body weight of the subject to be treated. In certain embodiments, at most 5×10⁵ fusion cells are administered per kg body weight of the subject to be treated. In certain embodiments, at most 10⁶ fusion cells are administered per kg body weight of the subject to be treated. In certain embodiments, at most 5×10⁶ fusion cells are administered per kg body weight of the subject to be treated. In certain embodiments, at most 10⁷ fusion cells are administered per kg body weight of the subject to be treated. In certain embodiments, at most 5×10⁷ fusion cells are administered per kg body weight of the subject to be treated. In certain embodiments, at most 10⁸ fusion cells are administered per kg body weight of the subject to be treated. In certain embodiments, at most 5×10⁸ fusion cells are administered per kg body weight of the subject to be treated. In certain embodiments, at most 10⁹ fusion cells are administered per kg body weight of the subject to be treated.

In certain embodiments, at least 10⁴ universal antigen-presenting cells are administered per kg body weight of the subject to be treated. In certain embodiments, at least 5×10⁴ universal antigen-presenting cells are administered per kg body weight of the subject to be treated. In certain embodiments, at least 10⁵ universal antigen-presenting cells are administered per kg body weight of the subject to be treated. In certain embodiments, at least 5×10⁵ universal antigen-presenting cells are administered per kg body weight of the subject to be treated. In certain embodiments, at least 10⁶ universal antigen-presenting cells are administered per kg body weight of the subject to be treated. In certain embodiments, at least 5×10⁶ universal antigen-presenting cells are administered per kg body weight of the subject to be treated. In certain embodiments, at least 10⁷ universal antigen-presenting cells are administered per kg body weight of the subject to be treated. In certain embodiments, at least 5×10⁷ universal antigen-presenting cells are administered per kg body weight of the subject to be treated. In certain embodiments, at least 10⁸ universal antigen-presenting cells are administered per kg body weight of the subject to be treated. In certain embodiments, at least 5×10⁸ universal antigen-presenting cells are administered per kg body weight of the subject to be treated. In certain embodiments, at least 10⁹ universal antigen-presenting cells are administered per kg body weight of the subject to be treated.

In certain embodiments, at most 10⁴ universal antigen-presenting cells are administered per kg body weight of the subject to be treated. In certain embodiments, at most 5×10⁴ universal antigen-presenting cells are administered per kg body weight of the subject to be treated. In certain embodiments, at most 10⁵ universal antigen-presenting cells are administered per kg body weight of the subject to be treated. In certain embodiments, at most 5×10⁵ universal antigen-presenting cells are administered per kg body weight of the subject to be treated. In certain embodiments, at most 10⁶ universal antigen-presenting cells are administered per kg body weight of the subject to be treated. In certain embodiments, at most 5×10⁶ universal antigen-presenting cells are administered per kg body weight of the subject to be treated. In certain embodiments, at most 10⁷ universal antigen-presenting cells are administered per kg body weight of the subject to be treated. In certain embodiments, at most 5×10⁷ universal antigen-presenting cells are administered per kg body weight of the subject to be treated. In certain embodiments, at most 10⁸ universal antigen-presenting cells are administered per kg body weight of the subject to be treated. In certain embodiments, at most 5×10⁸ universal antigen-presenting cells are administered per kg body weight of the subject to be treated. In certain embodiments, at most 10⁹ universal antigen-presenting cells are administered per kg body weight of the subject to be treated.

### 4.15 SCREENING METHODS

In certain embodiments, the invention provides screening methods for the identification of tumor-specific or tumor-associated antigens. The screening methods of the invention are based on the observation that DNA extracted from a tumor cell and transfected into a non-dendritic cell which in turn is fused with a dendritic cell can confer tumor-specific anti-tumor activity upon the fusion cells. Thus, without being bound by theory, the DNA encodes a tumor-specific or tumor associated antigen which is expressed by the fusion cell.

In certain embodiments, a cDNA library is generated from a tumor. In a specific embodiment, the cDNA library is generated from a single cell (see, e.g., Dulac and Axel, 1995, Cell 83(2):195-206). In a preferred embodiment, the cDNA library is generated from the same type of tumor as the type of tumor that is to be treated or prevented. Pools of cDNAs from the library are then introduced into non-dendritic cells which are subsequently used to generate different populations of fusion cells (i.e., each population of fusion cells contains a different pool of cDNAs). The different populations of fusion cells are tested for their anti-tumor activity as described in section 5. The cDNAs that were introduced into the population of fusion cells with the highest anti-tumor activity are then identified. In a specific embodiment all cDNAs of the library are sequenced and annotated. In another embodiments, only the cDNAs of the population of fusion cells with the highest anti-tumor activity are sequenced. In certain embodiments, the different pools are amplified separately to facilitate identification of the cDNAs. Once the cDNAs of the population of fusion cells with the highest anti-tumor activity are identified, smaller pools of cDNAs or individual cDNAs are introduced into the non-dendritic cells for generation of fusion cells and testing of the fusion cells for anti-tumor activity. The cDNAs that individually or in combination confer anti-tumor activity upon the fusion cells of the invention are identified as encoding tumor-specific or tumor-associated antigen.

### 4.16 KITS

The invention further provides kits for facilitating delivery of the immunotherapeutic composition according to the methods of the invention. The kits described herein may be conveniently used, e.g., in clinical settings to treat patients exhibiting symptoms of cancer or at risk of developing cancer. In one embodiment, for example, a kit is provided comprising, in one or more containers: a) a sample of a population of antigen presenting cells and b) a sample of non-dendritic cells. In certain embodiments, the antigen presenting cells that are provided in the kit are universal dendritic cells. Universal antigen presenting cells are prepared by recombinantly expressing co-stimulatory molecules (e.g., B7, ICAM-I and/or ICAM-II) in a cell. A universal antigen presenting cell can be prepared from any cell type. In certain embodiments, the universal antigen presenting cell is engineered to recombinantly express a cytokine, such as, but not limited to, IL-12. In certain embodiments, the antigen presenting cell is matched for major histocompatibility complex (MHC) with the subjected to be treated. For a more detailed description of universal antigen presenting cells, see section 4.7.

Kits of the invention can further comprise means for isolating pre-cancerous cells, tumor cells and/or cells infected with an infectious agent from a subject, such as materials for conducting a needle biopsy. Kits of the invention can further comprise means for extracting genomic DNA from a precancerous cell, a tumor cells, a cell infected with an infectious agent and/or an infectious agent. Kits of the invention can further comprise means for introducing the genomic DNA into the non-dendritic cells, such as, e.g., materials to conduct a lipofection.

Kits of the invention can further comprise means for fusing the non-dendritic cells into which the genomic DNA has been introduced and the antigen presenting cells. Means for fusion can be, but are not limited to, means for conducting electrofusion of the cells or means for fusing the cells using polyethylene glycol.

Kits of the invention can further comprise means for extracting mRNA from a precancerous cell, a tumor cells, a cell infected with an infectious agent and/or an infectious agent. Kits of the invention can further comprise means for synthesizing cDNA from the mRNA. Kits of the invention can further comprise means for introducing the cDNAs into the non-dendritic cells, such as, e.g., materials to conduct a lipofection. Kits of the invention can further comprise means for fusing the non-dendritic cells into which the cDNAs have been introduced and the antigen presenting cells. Means for fusion can be, but are not limited to, means for conducting electrofusion of the cells or means for fusing the cells using polyethylene glycol.

Other components of a kit of the invention may include instructions for its use in a method for treating or protecting against cancer or an infectious disease. An ampoule of sterile diluent can be provided so that the ingredients may be mixed prior to administration. In another embodiment the kit further comprises a cuvette suitable for electrofusion. In one embodiment, the antigen presenting cells are cryopreserved. In a further embodiment, the kit comprises a molecule that stimulates a humoral immune response and/or a cytotoxic T cell response. In a more preferred embodiment the stimulatory molecule is a cytokine such as, but not limited to interleukin-12.

In certain embodiments, a kit of the invention further contains cDNAs or expression vectors encoding tumor-associated antigens or tumor-associated epitopes. In certain embodiments, a kit of the invention further contains cDNAs or expression vectors encoding antigens or epitopes that are upregulated in the cancer to be treated compared to a noncancerous cell.

In certain embodiments, a kit of the invention comprises non-dendritic cells that contain one or more expression vectors encoding a tumor-associated antigen (see section 4.8). In certain embodiments, a kit of the invention includes means for obtaining non-dendritic cells from the subject to be treated, one or more expression vectors encoding tumor-associated antigens and/or means for transfecting the expression vector(s) into the non-dendritic cells.

In certain embodiments of the invention, a kit comprises a universal antigen presenting cell. In certain embodiments, a kit comprises a universal antigen presenting cell and means for: (i) obtaining a tumor cell, cell of a precancerous lesion, cell infected with an infectious agent, and/or infectious agent from a mammal; (ii) means for isolating genomic DNA from a cell; (iii) means for isolating mRNA from a cell; (iv) means for preparing cDNA from mRNA; (v) means for introducing mRNA, genomic DNA or cDNA into a cell; (vi) means for fusing cells; (vii) means for administering the universal antigen presenting cells or fusion cells to a subject; (iix) means for obtaining a non-dendritic cell from a mammal.

### 5. EXAMPLE I PREVENTION OF TUMOR DEVELOPMENT BY VACCINATION WITH FUSION CELLS

The present example demonstrates the prophylactic and therapeutic use of fusion cells formed by fusion of antigen presenting cells fused to non-dendritic cells that were transfected with genomic DNA extracted from different tumor cells.

Vaccination as well as treatment of mice with fusion cells formed between non-dentritic cells carrying genomic DNA of a tumor cell and antigen presenting cells inhibited the development tumors after challenge with different types of tumors. That is, the volume of tumors for treated mice was lower than that for untreated control mice.

Accordingly, these data support the prophylactic as well as the therapeutic efficacy of fusion cell vaccines comprising antigen presenting cells fused to non-dendritic cells carrying genomic DNA of a tumor cell. Finally, although the non-dendritic cells in the mouse model used in the present example were generated from tumor cells, the techniques described here may be applied to, and thus serve as a model for, the isolation of pre-cancerous non-dendritic cells, and their use to generate fusions for use in prophylactic and therapeutic vaccines against cancer.

### 5.1 MATERIALS AND METHODS

Mice, Tumor Models, and Cell Lines

Mouse fibroblast cell line NIH3T3 and mouse malignant tumor cell lines B16 and MC38 were obtained from the American Type Culture Collection (ATCC, Rockville, Md.). CT-2A glioma cells were kindly provided by Dr. Seyfried (13). These cell lines were maintained as monolayer cultures in Dulbecco's Modified Eagle Medium (DMEM; Cosmo Bio, Tokyo, Japan) containing 100 U/ml penicillin, 0.1 mg/ml streptomycin, and 10% heat-inactivated fetal bovine serum (FBS; BRL, Gaithersburg, Md.). Yac-1 cells, obtained from Riken Cell Bank (Tsukuba, Japan), were maintained in RPMI-1640 (BRL) supplemented with 10% FBS. Male C57BL/6J mice were purchased from Sankyo Laboratory, Shizuoka, Japan. Six week-old mice (body weight=25±2 g) were used in the experiments.

All of the experimental procedures were carried out in accordance with Jikei University guideline on animal welfare.

To induce tumors in the mice, B16 tumor cells or MC38 tumor cells, respectively, were injected into the left flanks of the mice subcutaneously.

Preparation of antigen presenting cells and fusion cells and fusion with non-dendritic cells.

Dendritic cells were prepared by the method described by Inaba et al. (Inaba et al., 1993, Generation of Large Numbers of Dendritic Cells from Mouse Bone Marrow Cultures Supplemented with GM-CSF. J Exp Med 176, 1693-1702; Inaba et al., 1993, Granulocytes, Macrophages and Dendritic Cells Arise from a Common Major Histocompatability Complex Class II-negative Progenitor in Mouse Bone Marrow, Proc Natl Acad Sci USA 90, 3038-3042). NIH 3T3 fibroblasts were co-transfected with genomic DNA extracted from B16 cells and pSV2-neo using lipofectamine.

Dendritic cells were fused with the transfected NIH3T3 fibroblasts according to Gong et al. (Gong et al., 1997, Induction of Antitumor Activity by Immunization with Fusion of Dendritic and Carcinoma Cells, Nat Med 3, 558-561).

More specifically, dendritic cells were isolated from bone marrow flushed from long bones of APC1309 mice, and red cells were lysed with ammonium chloride (Sigma, St. Louis, Mo.). Lymphocytes, granulocytes and T cells were depleted from the bone marrow cells and the cells were plated in 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 μg/ml streptomycin, 10 ng/ml recombinant murine granulocyte-macrophage colony stimulating factor (GM-CSF; Becton Dickinson, San Jose, Calif.) and 30 U/ml recombinant mouse interleukin-4 (IL-4; Becton Dickinson). On day 5 of culture, nonadherent and loosely adherent cells were collected and replated on 100-mm petri dishes (1×10⁶ cells/mi; 10 ml/dish). GM-CSF and IL-4 in RPMI medium were added to the cells and 1×10⁶ dendritic cells were mixed with 3×10⁶ tranfected NIH3T3 fibroblasts. After 48 hours, fusion was started by adding dropwise over 60 sec, 500 μl of a 50% solution of polyethylene glycol (PEG 1500; Sigma, St. Louis, Mo.). The fusion was stopped by stepwise addition of 30 ml. of serum-free RPMI medium. Fusion cells were plated in 100-mm petri dishes in the presence of GM-CSF and IL-4 in RPMI medium for 48 hr.

Transfection of NIH3T3 cells with genomic DNA

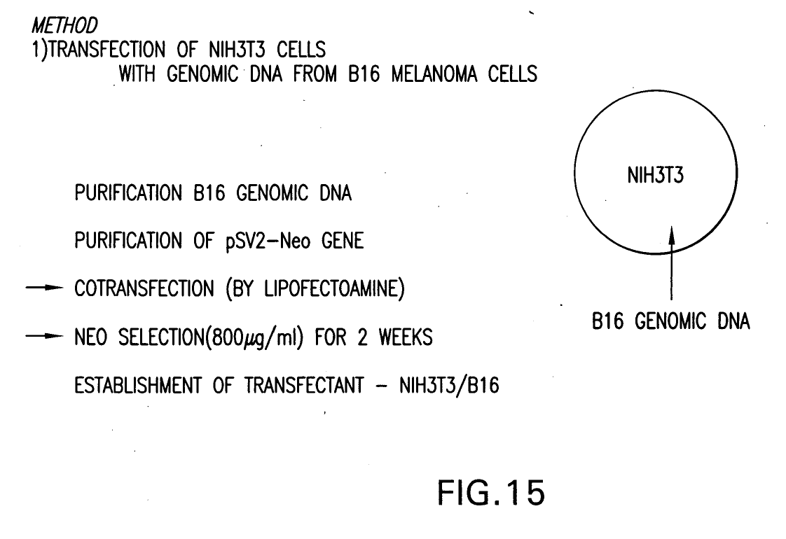

NIH3T3 cells were transfected with both genomic DNA and pSVNeo (kindly provided by Dr. Y. Manome; see also FIG. 15) using LipofectAMINE (BRL) according to the manufacturer's instructions. Briefly, 2 μg genomic DNA was mixed with 2 μg pSV2neo. The mixture was then mixed with LipofectAMINE and added to 1×10⁴ NIH3T3 cells. Forty-eight hours later, selection medium containing 800 μg/ml G418 (BRL) was added. Surviving colonies of transduced NIH-3T3 cells were expended and used for fusion. The transfectants were named NIH/B16, NIH/CT-2A, and NIH/NIH, respectively. Retroviral transfection of tumor cells

Treatment of mice and enumeration of the tumors

Fusion cells (2×10⁵/mouse) were injected into the tail vein of the subject mice. Mice were sacrificed at different time points after challenge with tumor cells and the tumor volume measured.

Assay of cytotoxicity of splenocvtes to B16 tumor cells.

Splenocytes were prepared by gentle disruption of spleen on a steel mesh and cultured in medium containing 50 U/ml of human recombinant IL-2 for 4 days and then examined for cytotoxic activity against B16 tumor cells. B16 tumor target cells, (1×10⁴ cells/well), were labeled with ⁵¹Cr, washed and incubated with the splenocytes at effector: target ratios ranging from 10:1 to 80:1 at 37° C. for 4 hours in 200 μl of RPMI-1640 medium supplemented with 10% heat inactivated fusion cells. After the cells were spun down by centrifugation, 100 μl of supernatant was collected for measurement of radioactivity. The percent specific ⁵¹Cr release was calculated according to the following formula: percent ⁵¹Cr release=100×(cpm experimental release−cpm spontaneous release)/(cpm maximum release−cpm spontaneous release). The maximum release was that obtained from target cells incubated with 0.33N HCl and spontaneous release was that obtained from target cells incubated without the effector cells.

### 5.2 RESULTS

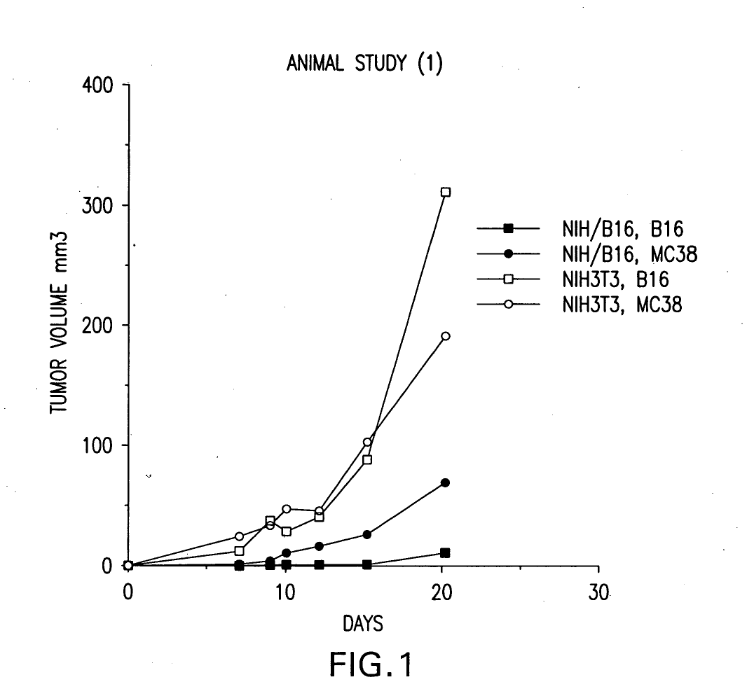

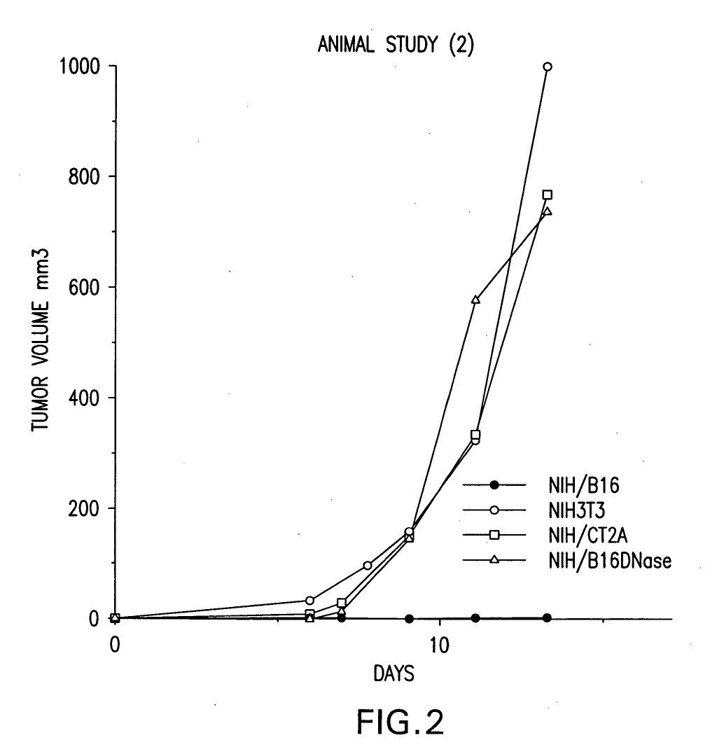

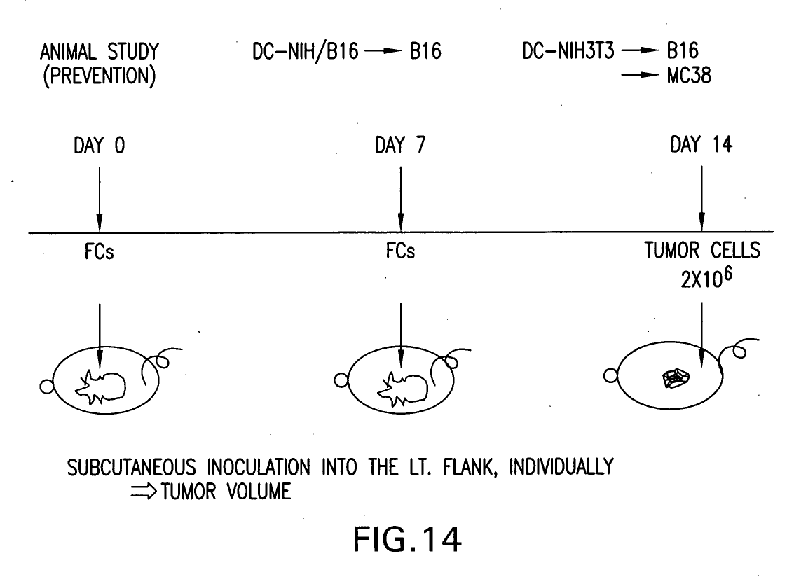

Mice were injected with fusion cells on 14 days and 7 days prior to challenge with the tumor (see FIG. 14). The fusion cells used were fusion cells of dendritic cells and NIH3T3 fibroblasts transfected with genomic DNA extracted from B16 tumor cells (NIH/B16); fusion cells of NIH3T3 fibroblasts that were not transfected with dendritic cells (NIH3T3); or fusion cells of dendritic cells and NIH3T3 fibroblasts transfected with genomic DNA extracted from CT-2A glioma cells (NIH/CT2A). FIG. 1 shows the tumor volumes after challenge with B16 or MC38 tumor cells, respectively, at day 0. The highest degree of prevention of tumor development was achieved when NIH/B16 were used to protect against challenge with B16 cells. FIG. 2 shows the tumor volumes after challenge with B16 tumor cells at day 0. Administering NIH3T3 or NIH/CT2A, respectively, did not result in protection against the tumor compared to NIH/B16 administration prior to challenge. Further, genomic DNA extracted from B16 tumor cells was treated with DNase before transfection of the DNA into NIH3T3 cells. Vaccination with fusion cells of dendritic cells and NIH3T3 fibroblasts that were transfected with genomic DNA that was treated with DNase (NIH/B16DNase) did not protect the mice from B16 tumor development.

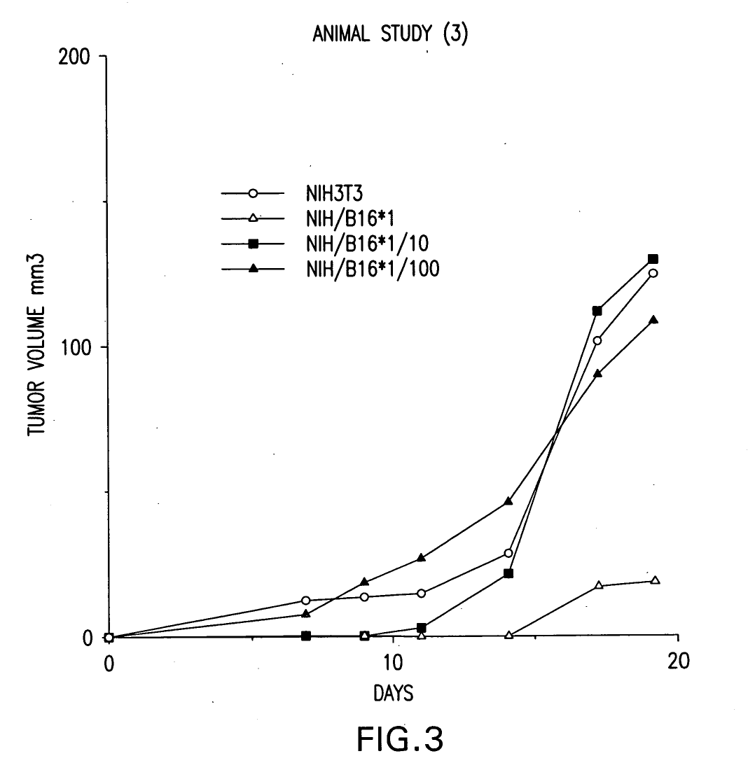

To estimate the amount of genomic DNA needed for the transfection of NIH3T3 fibroblasts, the fibroblasts were transfected with different amounts of genomic DNA extracted from B16 cells. FIG. 3 shows that transfection of 2 μg of genomic DNA per 10⁴ fibroblasts resulted in an effective treatment of the tumor. 10-fold or 100-fold lower amounts were ineffective in treating the tumor.

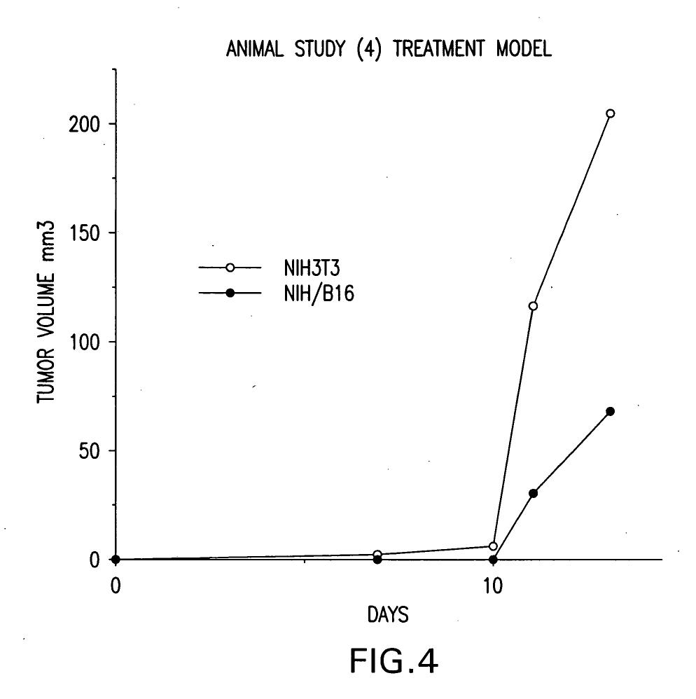

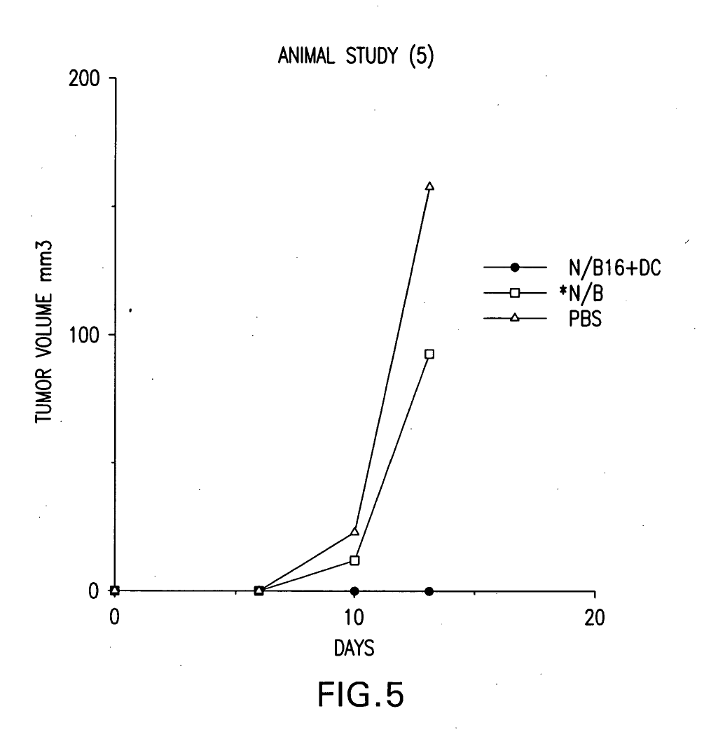

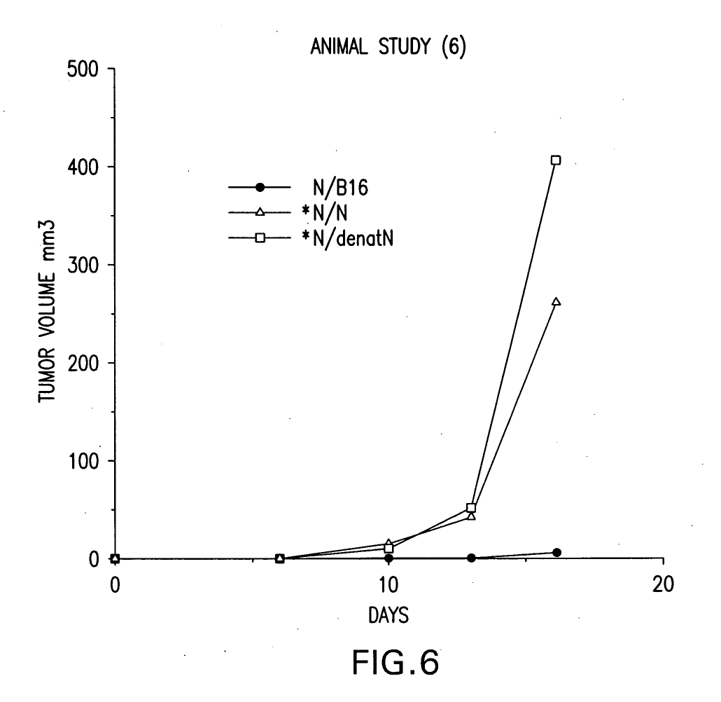

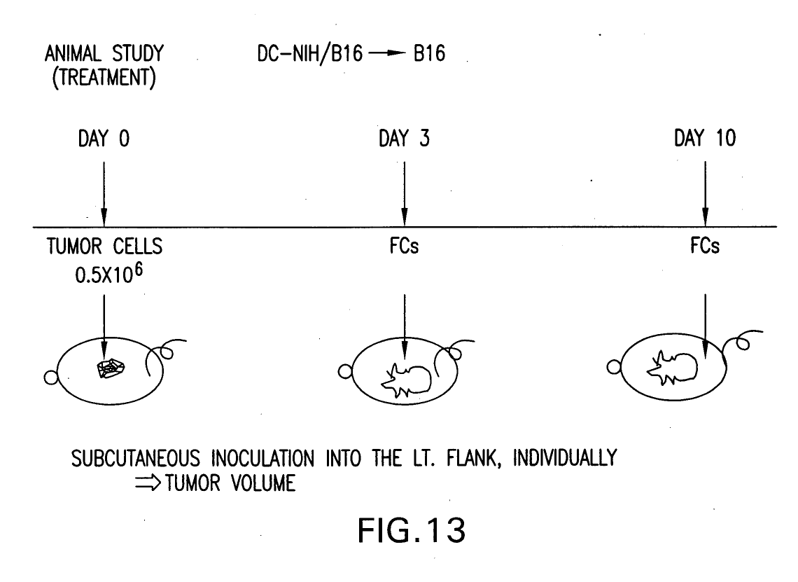

For the treatment of tumors, mice were challenged with B16 tumor cells on day 0 (FIG. 13). Fusion cells were administered on day 3 and day 10. FIG. 4 shows that treatment of B16 tumor bearing mice with NIH/B16 fusion cells is more effective at treating the tumor than treatment with fusion cells that were generated with untransfected NIH3T3 fibroblasts. The data presented in FIG. 5 demonstrate that NIH3T3 cells transfected with genomic DNA from B16 cells by themselves provide only slight tumor treatment. Fusion of the transfected NIH3T3 cells with dendritic cells, however, provides an effective treatment of the tumor. To assess the effect of heat-treatment of the DNA on the efficiency of the tumor treatment, genomic DNA extracted from B16 cells was denatured by heat prior to transfection of NIH3T3 cells with the genomic DNA. The data shown in FIG. 6 demonstrate that denaturing of the genomic DNA prior to transfection reduces the efficiency of the fusion cells that were generated from the transfected cells to treat the tumor. Cvtotoxic activity of splenocytes from fusion cell-immunized mice.

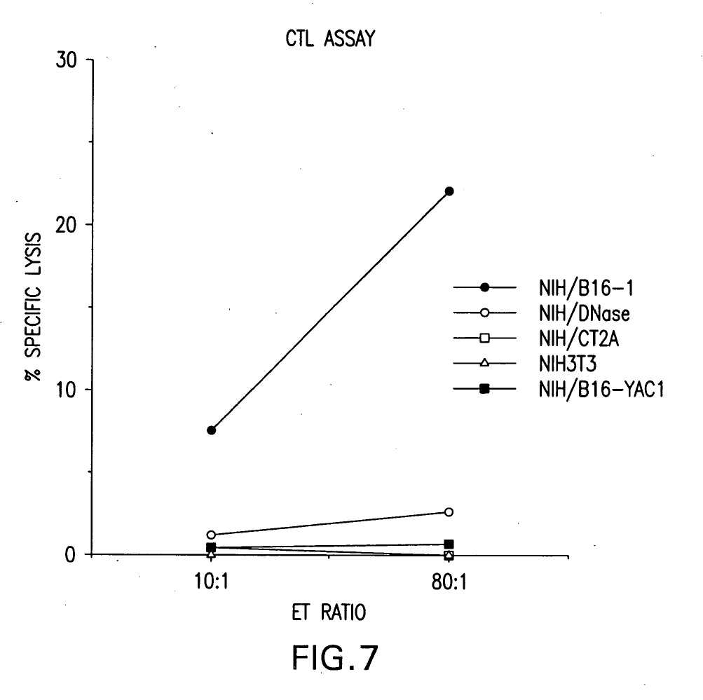

The data shown in FIG. 7 demonstrate that the cytotoxic activity of splenocytes isolated from mice that were treated with different types of fusion cells is highest if fusion cells of dendritic cells and NIH3T3 cells transfected with genomic DNA from B16 cells were used. That the cytotoxic activity of the splenocytes is specific to B16 cells is demonstrated by the fact that the cytotoxicity against YAC1 cells is drastically reduced compared to the cytotoxicity against B16 cells.

### 5.3 DISCUSSION

Dendritic cells, which are potent antigen presenting cells, have recently been utilized as an adjuvant for cancer immunotherapy. Cancer cells have acquired various strategies to evade the host immunosurveillance, hampering the development of effective immunotherapy. Gong et al. reported that inoculation of dendritic cells fused with tumor cell induced anti-tumor immunity in mice (Gong et al., 1997, Nat Med 3, 558-561). Successful clinical application of fused with tumor cell was also reported from Germany (Kugler et al., 2000, Nat Med 6, 332-336). It has been shown that intravenous administration of dendritic cells fused with APC1309 tumor cells of an established cell line from the colon cancer of APC1309 mice, prevented an increase in tumor number. In an APC1309 untreated mouse, about 100 tumors developed at 10 weeks of age in the whole gastrointestinal tract. Fusion cell-treatment decreased the number of tumors to one half of that in the untreated controls. Treatment with fusion cell in combination with interleukin-12 brought about a further reduction in the number of tumors observed. In fusion cell and interleukin-12-treated mice, the number of tumors was significantly lower at 10 weeks of age than at 6 weeks of age. Antitumor activity of interleukin-12 was reported by Brunda (Brunda et al., 2000, J Exp Med 178, 1223-1230) and Nastala (Nastala et al., 1994, J Immunol 153, 1697-1706). However the treatment of mice with interleukin-12 alone did not suppress the increase in the number of tumors significantly in the present study, suggesting that interleukin-12 enhances antitumor immunity induced by the treatment with fusion cells as discussed below.

It has been reported that CTL are the effector cells in antitumor immunity induced by dendritic cells loaded with tumor antigens (Paglia et al., 1996, J Exp Med 183: 317-322; Mayordomo et al., 1996, Nature Med 1(12), 1297-1302; Butterfield et al., 1998, J Immunol 161: 5607-13; Condon et al., 1996, Nature Medicine 2:, 1122-1128; Gong et al., 1997, Nat Med 3: 558-561.

In the present study, however, dendritic cells fused with NIH 3T3 cells that were transfected with genomic DNA of different tumors were shown to be capable of preventing and reducing the growth of tumors. The fusion cells were most effective if the genomic DNA that was introduced into the non-dendritic cells was extracted from the same type of tumor as the tumor to be treated or prevented. Thus, demonstrating that the specificity of the anti-tumor activity of the fusion cells of the invention depends on the source of the genomic DNA that was introduced into the non-dendritic cells that are used for the generation of the fusion cells.

DNase treatment of the genomic DNA before introducing the genomic DNA into the non-dendritic cells resulted in loss of the anti-tumor activity of the fusion cells. Further, a 10-fold reduction in the amount of genomic DNA being introduced into the non-dendritic cells also resulted in a loss of the anti-tumor activity of the fusion cells. Thus, the genomic DNA is an essential aspect of the methods of the present invention. Without being bound by theory, these results also demonstrate that the anti-tumor activity of the fusion cells of the present invention is not due to a contamination of the genomic DNA with tumor-specific antigens.

The present results demonstrate that immunization with dendritic cells fused with non-dendritic cells that harbor genomic DNA extracted from tumor cells is useful for prevention of tumor development and is also useful for the treatment of tumors.

### 6. EXAMPLE II: ANTITUMOR EFFECTS OF FUSIONS COMPOSED OF DENDRITIC CELLS AND FIBROBLASTS TRANSFECTED WITH GENOMIC DNA FROM TUMOR CELLS

### 6.1 Introduction

Dendritic cells (DCs) are professional antigen presenting cells (APCs) that have a unique potency for activating T cells. DCs express high levels of major histocompatibility complexes (MHC) and adhesion and costimulatory molecules (1). The efficient isolation and preparation of both human and murine DCs are now possible (2, 3). Therefore, a DC-based vaccine could potentially be used for the treatment of malignant tumors.

Since mature DCs lose the ability to take up antigens, use of mature DCs requires efficient methods for incorporating tumor associated antigens (TAAs) into DCs. To date, several methods using DCs for the induction of antitumor immunity have been investigated: DCs pulsed with proteins or peptides extracted from tumor cells (4), DCs transfected with genes encoding TAAs (5), DCs cultured with tumor cells (6), and DCs fused with tumor cells (fusion cells) (7-9). As we reported previously, systemic vaccination with recombinant interleukin 12 and fusion cells (FCs) containing dendritic and tumor cells prolonged the survival of tumor-bearing mice (7). Based on these experimental findings, clinical trials of vaccine therapy using FCs and recombinant human IL-12 against recurrent malignant tumors have begun. The advantages of this vaccination are that 1) FCs can be used to induce antitumor immunity against unknown TAAs and 2) the induction of autoimmune responses against normal cells can be avoided. On the other hand, the disadvantages are that I) cultured tumor cells are needed and 2) irradiated tumor cells may still exhibit tumorigenicity in vivo.

Classic studies indicate that transfection of genomic DNA can stably alter both the genotype and the phenotype of the cells that take up the exogenous DNA (10). In addition, it has been reported that immunotherapy using fibroblasts transfected with genomic DNA from tumor cells prolonged the survival of tumor bearing mice (11, 12). Based on these reports, we investigated antitumor effects of fusions containing autologous dendritic cells and allogeneic fibroblasts transfected with genomic DNA from tumor cells. The use of fibroblasts transfected with tumor cell genomic DNA overcomes the disadvantages of fusion cell therapy. That is, cultured tumor cells are not needed and, even if fibroblasts acquire tumorigenicity, allogeneic fibroblasts are rejected by the host thereby avoiding tumor formation. The present study demonstrates the antitumor effect and therapeutic efficacy of immunotherapy using FCs containing DCs and fibroblasts transfected with tumor-derived genomic DNA.

### 6.2 Materials and methods

Cell lines and animals

Mouse fibroblast cell line NIH3T3 and mouse malignant tumor cell lines B16 and MC38 were obtained from the American Type Culture Collection (ATCC, Rockville, Md.). CT-2A glioma cells were kindly provided by Dr. Seyfried (13). These cell lines were maintained as monolayer cultures in Dulbecco's Modified Eagle Medium (DMEM; Cosmo Bio, Tokyo, Japan) containing 100 U/ml penicillin, 0.1 mg/ml streptomycin, and 10% heat-inactivated fetal bovine serum (FBS; BRL, Gaithersburg, Md.). Yac-1 cells, obtained from Riken Cell Bank (Tsukuba, Japan), were maintained in RPMI-1640 (BRL) supplemented with 10% FBS. Male C57BU6J mice were purchased from Sankyo Laboratory, Shizuoka, Japan. Six week-old mice (body weight=25±2 g) were used in the experiments.

DNA extraction and transfection

Tumor cell genomic DNA was extracted from B16, CT-2A, or NIH3T3 cells using a DNA extraction kit (Qiagen, Hilden, Germany) according to the manufacturer's instructions. In some cases, genomic DNA was denatured by heating at 95° C. for 5 minutes and icing at 5° C. for 5 minutes or digested with DNase enzyme (TOYOBO, Tokyo, Japan; 1 U enzyme into 1 μg genomic DNA).

Transfection of genomic DNA into mouse fibroblasts

NIH3T3 cells were transfected with both genomic DNA and pSVNeo (kindly provided by Dr. Y. Manome) using LipofectAMINE (BRL) according to the manufacturer's instructions. Briefly, 2 μg genomic DNA was mixed with 2 μg pSV2neo. The mixture was then mixed with LipofectAMINE and added to 1×10⁴ NIH3T3 cells. Forty-eight hours later, selection medium containing 800 μg/ml G418 (BRL) was added. Surviving colonies of transduced NIH-3T3 cells were expended and used for fusion. The transfectants were named NIH/B16, NIH/CT-2A, and NIH/NIH, respectively. Retroviral transfection of tumor cells Green fluorescence protein gene plasmid pCMV-GFP (14) was kindly provided by Dr. Y. Manome. PAMPS1 retroviral producer cells (15) (kindly provided by Dr. Yoshimatsu) were transfected with pCMV-GFP (PAMP51/pCMV-GFP). The supernatant from PAMP51/pCMV-GFP was used to transfect MC38 target cells. After infection, MC38 cells were selected by using 800 μg/ml geneticine sulfate. Stable selection was completed after 14 days, and expression of the GFP was monitored by fluorescent microscopy.

Preparation of DCs

Separation of DCs from mouse bone marrow was performed as described previously (7). Briefly, the bone marrow was flushed from long bones of mice, and red cells were lysed with ammonium chloride (Sigma, St. Louis, Mo.). Lymphocytes and granulocytes were depleted from the bone marrow cells and the cells were plated on 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 μg/ml streptomycin (all from Sigma), 10 ng/ml recombinant murine granulocyte-macrophage colony stimulating factor (GM-CSF; Becton Dickinson, San Jose, Calif.), and 10 ng/ml recombinant mouse interleukin-4 (IL-4; Becton Dickinson). On day 5 of culture, nonadherent and loosely adherent cells were collected as DCs.

Fusions of dendritic and genetically-engineered NIH 3T3 cells

DCs were fused with genetically-engineered NIH3T3 cells as described previously (7). Briefly, 1×10⁶ DCs were mixed with 1×10⁶ genetically-engineered NIH3T3 cells (NIH/B16, NIH/CT-2A, or NIH/NIH). Then, fusion was started by adding 500 μl of a 50% solution of polyethylene glycol (PEG; Sigma) dropwise for 60 sec. The fusion was stopped by stepwise addition of serum-free RPMI medium. After washing three times with phosphate-buffered saline (PBS; Cosmo Bio), fusion cells (FCs) were plated on 100-mm petri dishes in the presence of GM-CSF and IL-4 in RPMI medium for 24 h. The various FCs were identified as FC/B16, FC/CT-2A, and FC/NIH, respectively.

Analysis of fusion efficiency

Fusion efficiency was investigated as follows. DCs and NIH3T3 cells were stained with fluorescein isothiocyanate-labeled anti-mouse CD80 monoclonal antibody (Pharmingen, San Diego, Calif., U.S.A) and PKH-26 (Sigma), respectively, according to the manufacturer's instructions. Immediately, those cells were fused as described above. FCs were resuspended in a buffer (1% BSA, 0.1% Sodium azide in PBS) and analyzed using a FACScan flow cytometer (Becton Dickinson, San Jose, Calif.). Double positive cells were determined to be fusion cells. Fusion efficiency was calculated as follows: Fusion efficiency=double positive cells/total cells×100 (%).

Animal models

FCs were washed twice with PBS, then suspended in PBS at a density of 1×10⁶/ml. FCs (3×10⁵) were subcutaneously (s.c.) inoculated into the flank of C57/6 mice on days 0 and 7. Subsequently, B16 tumor cells (1×10⁶) were inoculated s.c. into the flank on day 14.

Assay of cytolytic activity

The cytolytic activity of spleen cells (SPC) was tested in vitro using a standard ⁵¹Cr release assay (16). Single cell suspensions of SPC from individual mice were washed and resuspended in 10% FCS-RPMI at a density of 1×10⁷/ml in six-well plates (Falcon Labware, Lincoln Park, N.J.). After removing adherent cells, 10 U/ml of recombinant human IL-2 was added to the cultures every other day. Four days after culture initiation, cells were harvested and cytotoxic T cells (CTL) activity was determined. Target cells were labeled by incubation with ⁵¹Cr for 90 min at 37° C. and then co-cultured with effector lymphocytes for 4 hours. The effector:target ratio ranged from 10:1 to 80:1. All determinations were made in triplicate and percentage lysis was calculated using the formula: (experimental cpm—spontaneous cpm/maximum cpm—spontaneous cpm)×100%.

Antibody ablation studies

In vivo ablation of lymphocyte subsets was accomplished as previously described (16). Briefly, 3×10⁵ FCs were inoculated subcutaneously into the flank of C57/6 mice on days 0 and 7. B16 tumor cells (1×10⁶) were inoculated into the flank on day 14. Anti-asialo GM1 (Wako Pure Chemicals, Tokyo, Japan) was injected i.p. (0.5 mg/injection/mouse) on days −1, 3, 7, and 10.

### 6.3 Results

Transfer of tumor DNA leads to expression of encoded genes by MC38 cells

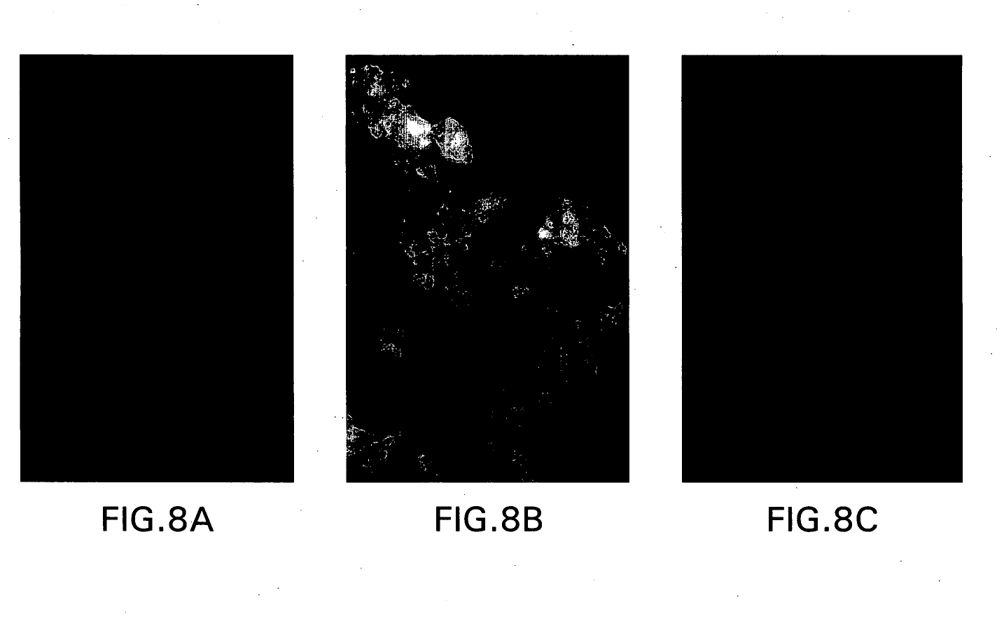

MC38 cells were first stably transfected with a retroviral construct containing a gene for GFP. Genomic DNA isolated from MC38/GFP cells was transferred to NIH3T3 cells, and after 48 h in culture, the transduced cells were tested for expression of GFP by fluorescence microscopy. Clusters of cells expressing GFP were present among transduced cells (FIG. 8-A). Flow cytometry confirmed that about 8% of the recipient cells expressed GFP (data not shown). Most of the MC38/GFP cells were positive for GFP (FIG. 8-B) and parental NIH3T3 cells were negative (FIG. 8-C). These data indicated that the phenotype of the recipient cells was altered by transfer of genomic DNA from the genetically-modified MC38 cells.

Fusion efficiency

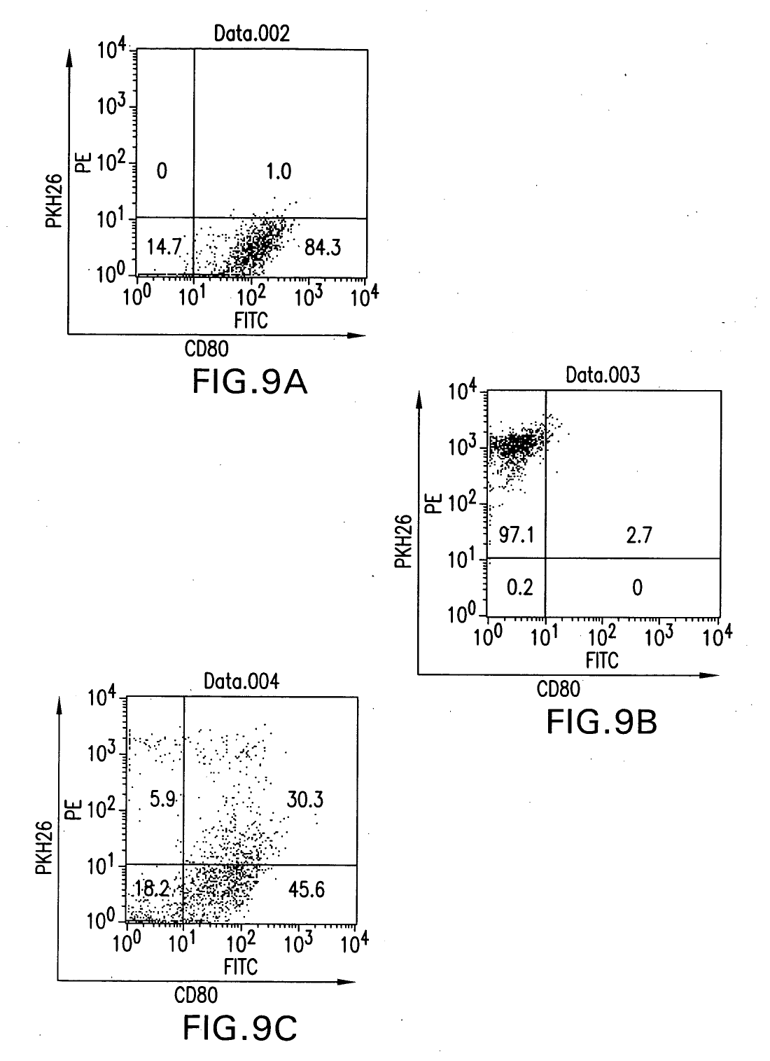

DCs and genetically-engineered fibroblasts were stained with anti-mouse CD80 monoclonal antibody and PKH-26, respectively, and fused by using PEG. Double positive cells were determined to be fusion cells. FIG. 9A shows that 85% of DCs were positive for anti-CD80 monoclonal antibody. More than 97% of NIHB16 cells were positive for PKH26 (FIG. 9B). The percentage of double positive cells was 30.3% (FIG. 9C). These experiments were repeated three times and the representative data were shown.

Immunization with FCs followed by tumor inoculation

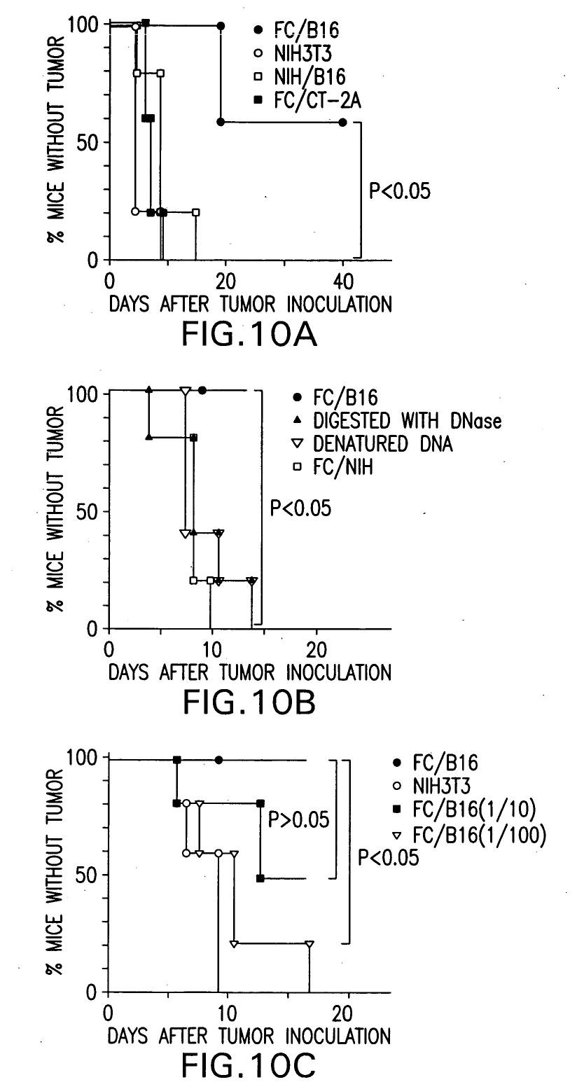

We examined the antitumor effects of prior immunization with FCs on subcutaneous tumors. FCs (3×10⁵ cells) containing DCs and NIH/B16 (FC/B16), 3×10⁵ NIH/B16 cells (not fused with DCs), 3×10⁵ FCs containing DCs and NIH/CT-2A (FC/CT-2A), or 3×10⁵ NIH3T3 cells as a control were injected s.c. into the flank of C57/6 mice on days 0 and 7 (n=5 in each group). On day 14, 1×10⁶ B16 cells were inoculated s.c. into the flank. The administration of FC/B16 prolonged the latency period before tumor appearance, while the administration of FC/CT-2A, NIH/B16 or NIH3T3 cells did not shorten the latency period before tumor appearance (p<0.05) (FIG. 10A). Vaccination with DCs alone, PBS alone, and a mixture of DCs and NIHB16 cells had no antitmor effect (data not shown).

Subsequently, we investigated whether the antitumor effect was dependent on the quality of genomic DNA transferred into NIH3T3 cells. We used FCs containing DCs and NIH/3T3 transfected with B16 genomic DNA digested with DNase or denatured DNA as a negative control. We also used FC/NIH (FCs containing DCs and NIH3T3 transfected with genomic DNA from NIH3T3). Immunization with these FCs did not shorten the latency period before tumor appearance (p<0.05) (FIG. 10B), indicating that the antitumor effect induced by FC/B16 was dependent on the quality of tumor derived genomic DNA transferred into NIH3T3 cells.

Furthermore, we also examined whether the antitumor effect was dependent on the dose of genomic DNA transfected into NIH/3T3 cells. NIH/3T3 cells (3×10⁵) were transfected with 2, 0.2, or 0.02 μg of genomic DNA from B16 cells. FCs containing DCs and each type of NIH/3T3 were identified as FC/high, FC/mid, and FC/low, respectively. FCs were injected s.c. into C57/6 mice on days 0 and 7 (n=5 in each group). On day 14, 1×10⁶ B16 cells were inoculated s.c. into the flank. No differences were observed in antitumor effects upon immunization with FC/mid or FC/low (p>0.05), whereas immunization with FC/high remarkably inhibited the growth of subcutaneous tumors (p<0.05), indicating that the antitumor effect induced by FC/B16 was DNA-dose dependent (FIG. 10C).

Induction of CTL activity

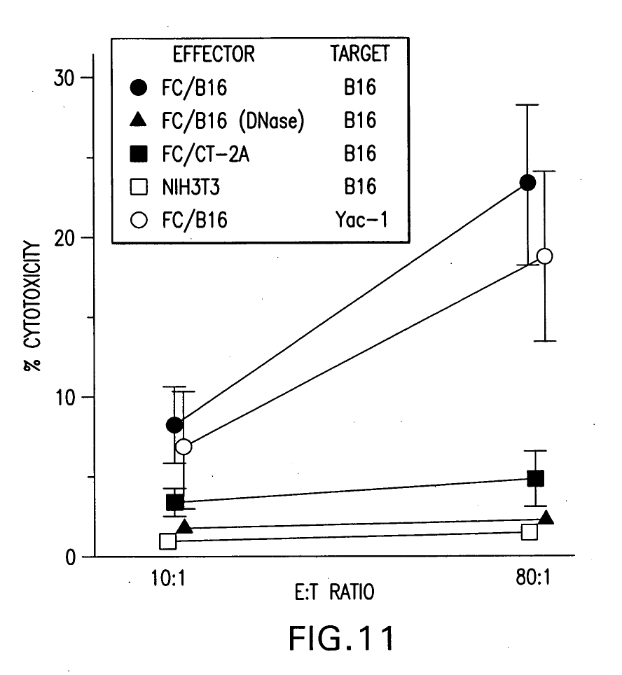

CTL activity was analyzed using a ⁵¹Cr release assay. After immunization with FCs (on days 0 and 7), SPC were separated from untreated mice and the mice were immunized with FCs on day 14. FIG. 11 shows that CTL activity on tumor cells from mice immunized with FC/B16 was considerably higher than in the control or other immunizations, and that antitumor activity on Yac-1 cells from mice immunized with FC/B16 increased. Antitumor activity on NIH/3T3 and CT-2A cells from mice immunized with FC/B16 did not increase (data not shown). These results suggest that vaccination with FC/B16 induced systemic antitumor immunity.

NK cells are required for antitumor effects of FCs

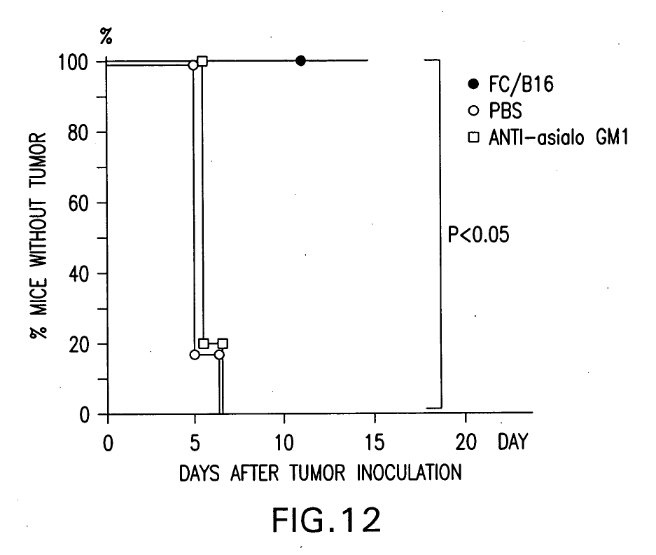

We examined the role of NK cells in the antitumor response generated by vaccination with FCs. NK cells were depleted by administering anti-asialo GM1 into mice given injections of B16 cells and FCs. On days 0 and 7, FC/B16 were subcutaneously inoculated into the flank. Subsequently, on day 14, B16 cells were inoculated into the same flank. Anti-asialo GM1 was injected i.p. on days −1, 3, 7, and 10. The antitumor effect was reduced in mice depleted of NK cells compared with the controls (n=5 in each group) (FIG. 12), suggesting that, in these experiments, NK cells are required for the antitumor effect induced by immunization with FC/B16.

### 6.4 Discussion

The results of the present study demonstrate that vaccination with FCs containing DCs and fibroblasts transduced with tumor-derived genomic DNA elicits antitumor immunity. To date, several methods using DCs for the induction of antitumor immunity have been investigated (4-8). The advantages of the present method are that 1) fibroblasts have no tumorigenicity, 2) allogeneic fibroblasts are rejected even if those cells acquire tumorigenicity, 3) cultured tumor cells are not needed, 4) antitumor immunity against unknown TAAs can be induced, and 5) several types of genetically-engineered fibroblasts can be prepared in advance (e.g. IL-12 transduced fibroblasts). Although fibroblasts exhibit no tumorigenicity, it remains unclear whether genetically-engineered fibroblasts are altered such that they acquire tumorigenicity. In the present study, allogeneic fibroblasts were used, and therefore, the host rejected the fibroblasts even if they had acquired tumorigenicity.

In previous studies, genetically-engineered DCs were used to elicit antitumor immunity. Ordinary DCs were adenovirally transduced with genes including those for IL-2, IL-12, GM-CSF, chemokines, and TAAs (5, 17-20). The disadvantage of this method was that transfection had to be performed each time DCs were used. On the other hand, although naive fibroblasts were used in the present study, fibroblasts transfected in advance with specific genes could be used instead of naive fibroblasts to enhance antitumor effects. The transduction of tumor-derived DNA into genetically-engineered fibroblasts such as IL-12 producing fibroblasts or CD40 ligand expressing fibroblasts, is expected to induce stronger antitumor immunity. We are currently investigating antitumor effects of fusions containing DCs and genetically-engineered fibroblasts transfected with both CD40L and tumor derived genomic DNA.

Studies have reported that genetically-engineered fibroblasts alone elicit antitumor immunity (11). In those studies, fibroblasts were used as APC. Schoenberger et al. reported that CTL induction required the engineered fibroblasts to express CD80 molecules (21). These fibroblasts were pulsed with tumor peptides. Therefore, the fibroblasts expressed both TAA and costimulatory molecule. On the other hand, in our study, fibroblasts were used as a transporter of genomic DNA from tumor cells, and both TAA and costimulatory molecules were expressed on DCs. As we reported previously, vaccination with genetically-engineered tumor cells that expressed both CD54 and CD80 inhibited the growth of tumors (22), suggesting that a single costimulatory molecule could not induce antitumor effects. DCs express high levels of MHC, adhesion and costimulatory molecules. Therefore, the FCs used in the present study elicited stronger antitumor immunity than genetically-engineered fibroblasts alone. Additionally, injection of allogeneic fibroblasts may induce an allogeneic reaction in the host, resulting in enhanced antitumor effects.

DCs can sensitize CD4⁺ T cells to specific antigens in a MHC-restricted manner. CD4⁺ T cells are critical in priming both cytotoxic T lymphocytes and antigen non-specific effector immune responses, implying that both CD4⁺ and CD8⁺ T cells are equally important in antitumor immunity. As reported previously, antitumor effects of cells fused with DCs and tumor cells are mediated via CD8³⁰ T cells, although the role of CD4³⁰ T cells and NK cells is less obvious (7). In the present study, CTL activity on both B16 cells and Yac-1 cells from mice immunized with FCs was considerably higher than in the control and otherwise immunized mice. In addition, the antitumor effect was reduced in mice depleted of NK cells. These results suggest that anti-tumor effects of FCs were mediated mainly via NK cells. Co-culture of the NK cells with DCs resulted in significant enhancement of NK cell cytotoxicity (23), indicating that the FCs, used in the present study, may stimulate NK cells directly. However, mice cured of their subcutaneous tumors by administration of FCs develop long-term systemic immunity against the parental tumor (data not shown). Additionally, vaccination with FC/CT-2A did not inhibit the growth of B16 cells, suggesting that the antitumor effect in this model is both tumor specific and non-specific and that T lymphocytes also play a role in antitumor effects induced by vaccination with FCs.

In conclusion, our data suggest that vaccination with FCs containing DCs and fibroblasts transfected with tumor-derived DNA can be used to treat malignant tumors in a mouse model. In the present study, allogeneic fibroblasts were used as a fusion partner. However, allogeneic tumor cells derived from the same organ may potentially be used instead of fibroblasts. The advantage of this method is that an antitumor immunity against common tumor antigens may be induced. Future research will focus on investigating antitumor effects of vaccination with fusion cells composed of syngeneic DCs and allogeneic tumor cells transfected with tumor-derived genomic DNA.

### REFERENCES

1. Steinman R M (1991) The dendritic cell system and its role in immunogenicity. Annu Rev Immunol 9; 271

2. Nestle F O, Alijagic S, Gilliet M, Sun Y, Grabbe S, Dummer R, Burg G, Schadendorf D (1998) Vaccination of melanoma patients with peptide- or tumor lysate-pulsed dendritic cells. Nat Med 4; 328

3. Reeves M E, Royal R E, Lam J S, Rosenberg S A, Hwu P (1996) Retroviral transduction of human dendritic cells with a tumor-associated antigen gene. Cancer Res 56; 5672

4. Zitvogel L, Mayordomo J I, Tjandrawan T, DeLeo A B, Clarke M R, Lotze M T, Storkus W J (1996) Therapy of murine tumors with tumor peptide-pulsed dendritic cells: dependence on T cells, B7 costimulation, and T helper cell 1-associated cytokines. J Exp Med 183; 87

5. Tuting T, Wilson C C, Martin D M, Kasamon Y L, Rowles J, Ma D I, Slingluff C L, Wagner S N, van der Bruggen P, Baar J, Lotze M T, Storkus W J (1998) Autologous human monocyte-derived dendritic cells genetically modified to express melanoma antigens elicit primary cytotoxic T cell responses in vitro: enhancement by cotransfection of genes encoding the Th1-biasing cytokines IL-12 and IFN-alpha. J Immunol 160; 1139

6. Celluzzi C M, Falo L D (1998) Physical interaction between dendritic cells and tumor cells results in an immunogen that induces protective and therapeutic tumor rejection. J Immunol 160; 3081

7. Akasaki Y, Kikuchi T, Homma S, Abe T, Kufe D, Ohno T (2001) Antitumor effect of immunizations with fusions of dendritic and glioma cells in a mouse brain tumor model. J Immunother 24; 106

8. Gong J, Chen D, Kashiwaba M, Kufe D (1997) Induction of antitumor activity by immunization with fusions of dendritic and carcinoma cells. Nat Med 3; 558

9. Gong J, Chen D, Kashiwaba M, Li Y, Chen L, Takeuchi H, Qu H, Rowse G J, Gendler S J, Kufe D (1998) Reversal of tolerance to human MUC1 antigen in MUC1 transgenic mice immunized with fusions of dendritic and carcinoma cells. Proc Natl Acad Sci USA 95; 6279

10. Wigler M, Pellicer A, Silverstein S, Axel R (1978) Biochemical transfer of single-copy eucaryotic genes using total cellular DNA as donor. Cell 14; 725

11. de Zoeten E, Carr-Brendel V, Markovic D, Taylor-Papadimitriou J, Cohen E P (1999) Treatment of breast cancer with fibroblasts transfected with DNA from breast cancer cells. J Immunol 162; 6934

12. Whiteside T L, Gambotto A, Albers A, Stanson J, Cohen E P (2002) Human tumor-derived genomic DNA transduced into a recipient cell induces tumor-specific immune responses ex vivo. Proc Natl Acad Sci USA 99; 9415

13. Ecsedy J A, Manfredi M G, Yohe H C, Seyfried T N (1997) Ganglioside biosynthetic gene expression in experimental mouse brain tumors. Cancer Res 57; 1580

14. Housey G M, Johnson M D, Hsiao W L, O'Brian C A, Murphy J P, Kirschmeier P, Weinstein I B (1988) Overproduction of protein kinase C causes disordered growth control in rat fibroblasts. Cell 52; 343

15. Yoshimatsu T, Tamura M, Kuriyama S, Ikenaka K (1998) Improvement of retroviral packaging cell lines by introducing the polyomavirus early region. Hum Gene Ther 9; 161

16. Kikuchi T, Joki T, Saitoh S, Hata Y, Abe T, Kato N, Kobayashi A, Miyazaki T, Ohno T (1999) Anti-tumor activity of interleukin-2-producing tumor cells and recombinant interleukin 12 against mouse glioma cells located in the central nervous system. Int J Cancer 80; 425

17. Ribas A, Bui L A, Butterfield L H, Vollmer C M, Jilani S M, Dissette V B, Glaspy J A, McBride W H, Economou J S (1999) Antitumor protection using murine dendritic cells pulsed with acid-eluted peptides from in vivo grown tumors of different immunogenicities. Anticancer Res 19; 1165

18. Kaneko K, Wang Z, Kim S H, Morelli A E, Robbins P D, Thomson A W (2003) Dendritic cells genetically engineered to express IL-4 exhibit enhanced IL-12p70 production in response to CD40 ligation and accelerate organ allograft rejection. Gene Ther 10; 143

19. Nakamura M, Iwahashi M, Nakamori M, Ueda K, Matsuura I, Noguchi K, Yamaue H (2002) Dendritic cells genetically engineered to simultaneously express endogenous tumor antigen and granulocyte macrophage colony-stimulating factor elicit potent therapeutic antitumor immunity. Clin Cancer Res 8; 2742

20. Nelson W G, Simons J W, Mikhak B, Chang J F, DeMarzo A M, Carducci M A, Kim M, Weber C E, Baccala A A, Goeman M A, Clift S M, Ando D G, Levitsky H I, Cohen L K, Sanda M G, Mulligan R C, Partin A W, Carter H B, Piantadosi S, Marshall F F (2000) Cancer cells engineered to secrete granulocyte-macrophage colony-stimulating factor using ex vivo gene transfer as vaccines for the treatment of genitourinary malignancies. Cancer Chemother Pharmacol 46 Suppl; S67

21. Schoenberger S P, Jonges L E, Mooijaart R J, Hartgers F, Toes R E, Kast W M, Melief C J, Offringa R (1998) Efficient direct priming of tumor-specific cytotoxic T lymphocyte in vivo by an engineered APC. Cancer Res 58; 3094

22. Joki T, Kikuchi T, Akasaki Y, Saitoh S, Abe T, Ohno T (1999) Induction of effective antitumor immunity in a mouse brain tumor model using B7-1 (CD80) and intercellular adhesive molecule 1 (ICAM-1; CD54) transfection and recombinant interleukin 12. Int J Cancer 82; 714

23. Yu Y, Hagihara M, Ando K, Gansuvd B, Matsuzawa H, Tsuchiya T, Ueda Y, Inoue H, Hotta T, Kato S (2001) Enhancement of Human Cord Blood CD34(+) Cell-Derived NK Cell Cytotoxicity by Dendritic Cells. J Immunol 166; 1590

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

1. A method of treating or preventing cancer in a mammal, said method comprising administering to a mammal in need of said treatment or prevention an effective amount of fusion cells, wherein a fusion cell (i) is formed by the fusion of an antigen presenting cell and a non-dendritic cell, wherein the non-dendritic cell comprises genomic DNA of a cancer cell and wherein said genomic DNA encodes at least one antigen having the antigenicity of an antigen associated with said cancer, and (ii) shares at least one MHC class I allele with said mammal.

2. A method of treating or preventing cancer in a mammal, said method comprising administering to a mammal in need of said treatment or prevention an effective amount of universal antigen presenting cells, wherein a universal antigen presenting cell (i) has been engineered to recombinantly express one or more costimulatory molecules selected from the group consisting of: ICAM-I, ICAM-II, B7, and LFA-3; (ii) comprises genomic DNA of a cancer cell and wherein said genomic DNA encodes at least one antigen having the antigenicity of an antigen associated with said cancer; and (iii) shares at least one MHC class I allele with said mammal.

3. The method of claim 1, wherein the genomic DNA is isolated from a cancer cell that is of the same type as the cancer to be prevented in the mammal.

4. The method of claim 1, wherein the genomic DNA is isolated from a cancer cell that is obtained from the cancer to be prevented in the mammal.

5. A method of treating a pre-cancerous lesion in a mammal, said method comprising administering to a mammal in need of said treatment a therapeutically effective amount of fusion cells, wherein a fusion cell (i) is formed by the fusion of an antigen presenting cell and a non-dendritic cell, wherein the non-dendritic cell comprises genomic DNA of a cell of a pre-cancerous lesion and wherein said genomic DNA encodes at least one antigen having the antigenicity of an antigen associated with said pre-cancerous lesion, and (ii) shares at least one MHC class I allele with said mammal.

6. A method of treating a pre-cancerous lesion in a mammal, said method comprising administering to a mammal in need of said treatment a therapeutically effective amount of universal antigen presenting cells, wherein a universal antigen presenting cell (i) has been engineered to recombinantly express one or more costimulatory molecules selected from the group consisting of: ICAM-I, ICAM-II, B7, and LFA-3; (ii) comprises genomic DNA of a cell of a pre-cancerous lesion and wherein said genomic DNA encodes at least one antigen having the antigenicity of an antigen associated with said pre-cancerous lesion; and (iii) shares at least one MHC class I allele with said mammal.

7. The method of claim 2, wherein the universal antigen presenting cell recombinantly expresses at least one MHC class I allele of said mammal.

8. The method of claim 2, wherein the universal antigen presenting cell is generated from a cell allogeneic to said mammal.

9. The method of claim 2, wherein the universal antigen presenting cell is generated from a cell syngeneic to said mammal.

10. The method of claim 5, wherein the genomic DNA is isolated from a cell of a pre-cancerous lesion that is of the same type as the pre-cancerous lesion to be treated in the mammal.

11. The method of claim 5, wherein the genomic DNA is isolated from a cell of a pre-cancerous lesion that is isolated from the pre-cancerous lesion to be treated in the mammal.

12. The method of claim 1, wherein the antigen presenting cell is a dendritic cell.

13. The method of claim 1, further comprising administration of a molecule that stimulates a humoral immune response or a cytotoxic T cell immune response.

14. The method of claim 13, wherein said molecule is a cytokine.

15. The method of claim 14, wherein the cytokine is interleukin-12.

16. The method of claim 1, wherein the dendritic cell is obtained from human blood monocytes.

17. The method of claim 1, wherein said antigen presenting cells are autologous to said mammal.

18. The method of claim 1, wherein said antigen presenting cells are allogeneic to the mammal.

19. The method of claim 1, wherein the non-dendritic cell is autologous to the mammal.

20. The method of claim 1, wherein the non-dendritic cell is allogeneic to the mammal.

21. The method of claim 1, wherein said antigen presenting cells are allogeneic to the mammal and wherein said non-dendritic cells have the same class I MHC haplotype as the mammal.

22. The method of claim 1, wherein said mammal is a human.

23. The method of claim 1, wherein said mammal is selected from the group consisting of a cow, a horse, a sheep, a pig, a fowl, a goat, a cat, a dog, a hamster, a mouse and a rat.

24. The method of claim 1, wherein said cancer is selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenstrbm's macroglobulinemia, and heavy chain disease.

25. (Canceled)

26. A method for fusing human antigen presenting cells and non-dendritic human cells comprising subjecting a population of antigen presenting cells and a population of non-dendritic cells to conditions that promote cell fusion, wherein the non-dendritic cells comprise genomic DNA of a tumor cell, wherein the genomic DNA encodes at least one antigen associated with the tumor cell.

27.-30. (canceled)

31. A fusion cell of an antigen presenting cell and a nondendritic cell, wherein the fusion cell comprises genomic DNA of a tumor cell, wherein the genomic DNA of the tumor cell encodes encodes at least one antigen associated with the tumor cell.

32. A kit comprising, in one or more containers, (i) a population of antigen presenting cells; (ii) a population of non-dendritic cells; and (iii) instructions for fusing said antigen presenting cells with the non-dendritic cells for administration to a mammal in need thereof.

33.-38. (canceled)

39. A pharmaceutical composition comprising a fusion cell comprising an antigen presenting cell and a non-dendritic cell, wherein the non-dendritic cell comprises genomic DNA of a tumor cell, wherein the genomic DNA encodes at least one antigen associated with the tumor cell.

40.-44. (canceled)

45. A cell for treating a cancer in a mammal, comprising the steps of engineering a cell to recombinantly express
(i) one or more costimulatory molecules selected from the group consisting of:
ICAM-I, ICAM-II, B7, and LFA-3;
(ii) at least one MHC class I allele of said mammal; and
(iii) at least one antigen that is associated with said cancer.

46.-47. (canceled)

48. A pharmaceutical composition comprising the cell of claim 45 and a pharmaceutically acceptable carrier.

49. A method of treating or preventing cancer in a mammal, said method comprising administering to a mammal in need of said treatment or prevention an effective amount of fusion cells, wherein a fusion cell (i) is formed by the fusion of an antigen presenting cell and a non-dendritic cell, wherein the non-dendritic cell comprises one or more cDNAs wherein at least one cDNA encodes an antigen having the antigenicity of an antigen associated with said cancer, and (ii) shares at least one MHC class I allele with said mammal.

50. A method of treating or preventing cancer in a mammal, said method comprising administering to a mammal in need of said treatment or prevention an effective amount of universal antigen presenting cells, wherein a universal antigen presenting cell (i) has been engineered to recombinantly express one or more costimulatory molecules selected from the group consisting of: ICAM-I, ICAM-II, B7, and LFA-3; (ii) comprises one or more cDNAs wherein at least one cDNA encodes an antigen having the antigenicity of an antigen associated with said cancer, and (iii) shares at least one MHC class I allele with said mammal.

51.-54. (canceled)

55. A method of treating a pre-cancerous lesion in a mammal, said method comprising administering to a mammal in need of said treatment a therapeutically effective amount of fusion cells, wherein a fusion cell (i) is formed by the fusion of an antigen presenting cell and a non-dendritic cell, wherein the non-dendritic cell comprises one or more cDNAs wherein at least one cDNA encodes an antigen having the antigenicity of an antigen associated with said pre-cancerous lesion, and (ii) shares at least one MHC class I allele with said mammal.

56. A method of treating or preventing a pre-cancerous lesion in a mammal, said method comprising administering to a mammal in need of said treatment or prevention an effective amount of universal antigen presenting cells, wherein a universal antigen presenting cell (i) has been engineered to recombinantly express one or more costimulatory molecules selected from the group consisting of: ICAM-I, ICAM-II, B7, and LFA-3; (ii) comprises one or more cDNAs wherein at least one cDNA encodes an antigen having the antigenicity of an antigen associated with said pre-cancerous lesion, and (iii) shares at least one MHC class I allele with said mammal.

57.-74. (canceled)

75. A method for fusing human antigen presenting cells and non-dendritic human cells comprising subjecting a population of antigen presenting cells and a population of non-dendritic cells to conditions that promote cell fusion, wherein the non-dendritic cells comprise one or more cDNAs wherein at least one cDNA encodes an antigen associated with a cancer.

76.-78. (canceled)

79. A fusion cell of an antigen presenting cell and a non-dendritic cell, wherein the fusion cell comprises a cDNA encoding an antigen associated with a tumor cell.

80.-88. (canceled)

89. A pharmaceutical composition comprising a fusion cell comprising an antigen presenting cell and a non-dendritic cell, wherein the non-dendritic cell comprises at least one cDNA encoding an antigen associated with a tumor cell.

90.-98. (canceled)

99. A method for expanding antigen-specific immune effector cells, wherein the method comprises incubating an immune effector cell with a fusion cell, wherein the fusion cell is formed by the fusion of an antigen presenting cell and a non-dendritic cell, wherein the non-dendritic cell comprises genomic DNA of a cancer cell or of a cell of a precancerous lesion and wherein said genomic DNA encodes at least one antigen having the antigenicity of an antigen associated with said cancer or said precancerous lesion.

100. A method for expanding antigen-specific immune effector cells, wherein the method comprises incubating an immune effector cell with a universal antigen presenting cell, wherein a universal antigen presenting cell (i) has been engineered to recombinantly express one or more costimulatory molecules selected from the group consisting of: ICAM-I, ICAM-II, B7, and LFA-3; and (ii) comprises genomic DNA of a cancer cell or a cell of a precancerous lesion and wherein said genomic DNA encodes at least one antigen having the antigenicity of an antigen associated with said cancer or with said precancerous lesion.

101. A method for expanding antigen-specific immune effector cells, wherein the method comprises incubating an immune effector cell with a fusion cell, wherein a fusion cell is formed by the fusion of an antigen presenting cell and a non-dendritic cell, wherein the non-dendritic cell comprises one or more cDNAs wherein at least one cDNA encodes an antigen having the antigenicity of an antigen associated with a cancer or a precancerous lesion.

102. A method for expanding antigen-specific immune effector cells, wherein the method comprises incubating an immune effector cell with a universal antigen presenting cell, wherein a universal antigen presenting cell (i) has been engineered to recombinantly express one or more costimulatory molecules selected from the group consisting of: ICAM-I, ICAM-II, B7, and LFA-3; and (ii) comprises one or more cDNAs wherein at least one cDNA encodes an antigen having the antigenicity of an antigen associated with a cancer or a precancerous lesion.

103.-104. (canceled)
